(19) United States
(12) Patent Application Publication
(10) Pub. No.: US 2026/0357119 A1
(43) Pub. Date: Nov. 12, 2026

(54) FLUORINE SUBSTITUTED SULFUR COMPOUNDS, AND COMPOSITIONS, METHODS AND USES INCLUDING SAME

(71) Applicant: Solstice Advanced Materials US, Inc., Morris Plains, NJ (US)

(72) Inventors: Vinayak Krishnamurti, Morris Plains, NJ (US); Colby Barrett, Morris Plains, NJ (US); Zachary Fellin, Morris Plains, NJ (US); Dimitrios Papanastasiou, Morris Plains, NJ (US); Chris Roger, Morris Plains, NJ (US); Kaimi Gao, Morris Plains, NJ (US)

(21) Appl. No.: 19/662,079

(22) Filed: Apr. 29, 2026

Related U.S. Application Data
(60) Provisional application No. 63/796,561, filed on Apr. 29, 2025.

(51) Int. Cl. C09K 5/04; C07C 323/03; C07C 323/05

(52) U.S. Cl. CPC C09K 5/048; C07C 323/03; C07C 323/05

(57) ABSTRACT

Heat transfer composition for transferring heat and/or energy to and/or from an article, device or fluid, wherein said heat transfer composition comprises one or more compounds of
Formula I:

F₅S—C₂H_{X}—SF₅ Formula I

where x is 0, 2 or 4.

### CROSS REFERENCE TO RELATED APPLICATIONS

The present application is related to and claims the priority benefit of U.S. Provisional Application No. 63/796,561, which was filed Apr. 29, 2025 and which is incorporated herein by reference in its entirety.

### FIELD

The present invention relates to fluorine substituted sulfur compounds, including novel fluorine substituted sulfur compounds, to compositions containing the same, and to methods and uses of such compounds and compositions in heat transfer applications, including but not limited to high temperature heat pumps, organic Rankine Cycle working fluids, temperature control in manufacture of electronic equipment; thermal management of operating electronic devices and power systems, including immersion cooling, and avionic and military cooling.

### BACKGROUND

Certain halogen substituted sulfur compounds are known to exist. For example, U.S. Pat. No. 6,546,740 discloses refrigerant mixtures that may include trifluoromethylsulfur pentafluoride (CF₃SF₅), and U.S. Pat. No. 5,626,790 discloses refrigerant mixtures that may include difluoromethyl sulfur pentafluoride (SF₅CF2H) and pentafluoroethyl sulfur pentafluoride (SF₅C₂F₅).

Applicants have come to appreciate that many challenging issues are associated with the development of new compounds and compositions for use in many important applications. For example, in portable and hand-held electronic devices, the desire to miniaturize while adding functionality increases the thermal power density of the device while in operation, thus making cooling of the electronics components within such devices, including the batteries, more challenging. As a general rule, increases in computational power within desktop computers, data centers, telecommunications centers, and the like result in an increase in the heat output when such devices are operating, again making thermal management of such electronic devices increasingly important and increasingly more difficult and demanding. Other examples of thermal management challenges occur as a result of the increasing use of electric vehicles, including particularly, cars, trucks, motorcycles and the like. In electric vehicles the thermal management function is especially important and challenging for several reasons, including the criticality of cooling and/or heating the batteries to be within a relatively narrow temperature range and in a way that is reliable, efficient, and safe, and the challenge to provide effective thermal management of batteries is becoming greater as the demand for battery-operated vehicles with greater range and faster charging increases.

The efficiency and effectiveness of batteries, especially the batteries that provide the power in electric vehicles, is a function of the temperature at which they operate. Thus, a thermal management system must frequently be able to do more than simply remove heat from the battery during operation and/or charging—it must be able to effect cooling in a relatively narrow temperature range using equipment that is as low cost as possible and as light weight as possible. This results in the need for a heat transfer fluid in such systems that possesses a difficult-to-achieve combination of physical and performance properties. Thus, the art has experienced difficulty in discovering and developing/obtaining compounds and/or compositions effective in such systems, not only from a thermal performance standpoint, but also from a myriad of other standpoints, including environmental, safety (flammability and toxicity), dielectric properties, and others.

There has recently been an increased interest by several government bodies in compounds that are per- and poly-fluoroalkyl substances (PFAS). For example, the government agency known as the Organization for Economic Co-operation and Development (OECD) has defined “PFAS” as fluorinated substances that contain at least one fully fluorinated methyl or methylene carbon atom (without any H/Cl/Br/I atom attached to it), and with a few noted exceptions, any chemical with at least a perfluorinated methyl group (—CF₃) or a perfluorinated methylene group (—CF₂—) is considered to be a PFAS molecule.¹ For convenience, a compound that is not within the definition of PFAS as presented above is referred to herein for convenience as “non-PFAS.”¹ OECD (2021), Reconciling Terminology of the Universe of Per- and Polyfluoroalkyl Substances: Recommendations and Practical Guidance, OECD Series on Risk Management, No. 61, OECD Publishing, Paris.

For applications involving heat transfer with electronic components, including immersion cooling of electronic components, the dielectric constant of the heat transfer fluid that is in contact with the electronic component can be an important property since the fluid used in such systems must be electrically compatible with the electronic device or component (such as batteries), especially while the battery or device is in operation. In general, this means the fluid must not only be non-flammable, it must also have a low electrical conductivity and a high level of stability while in contact with the battery or other electronic component while the component is operating and at the relatively high temperatures existing during operation. Applicants have come to appreciate the desirability of such properties even in indirect cooling of operating electronic devices and batteries because leakage of any such fluid may result in contact with operating electronic components.

Perfluorinated compounds have heretofore frequently been used as part of a heat transfer system in many of these demanding applications. For example, a coolant which has been commonly used for indirect battery cooling is a water/glycol combination, wherein the water/glycol combination is carried through a pipe for indirect heat transfer of heat from a battery compartment that is filled with a sealing liquid that can be silicone oil, transformer oil, rchlorofluorocarbons, fluorohydrocarbons, chlorohydrocarbons and hydrofluoroethers. See, for example, US 2018/0191038.

Vapor phase soldering is another example of a process that utilizes heat transfer fluids. In this application, high temperatures are used and accordingly the heat transfer fluid must be suitable for high temperature exposure (e.g., up to 250 °C) Currently, perfluoropolyethers (PFPE) are commonly used as the heat transfer fluids in this application. Although many PFPEs have adequate thermal stability for these high temperatures, they are environmentally persistent with extremely long atmospheric lifetimes which, in turn, gives rise to high global warming potentials (GWPs).

Thus, applicants have come to appreciate the interest in heat transfer fluids and for heat transfer methods and systems, which with are low GWP, low ODP, and non-PFAS, and are also preferably non-flammable, have acceptable toxicity, have excellent thermal properties that provide effective cooling and/or heat, including at relatively high temperatures and/or for use in operating electronic components at high temperatures and/or in a relatively narrow temperature range with equipment that preferably is low cost, reliable and light weight, among other uses. Thus, for example, applicants have found that fluids that have relatively low boiling points (e.g., below 50 °C) are not desirable in many applications since the use of such fluids will tend to increase the cost and/or weight of the cooling equipment for many battery and/or electronic cooling applications, and/or do not have adequate heat transfer performance, and/or may also decrease reliability, as explained hereinafter.

The Rankine cycle is the standard thermodynamic cycle in general use for electric power generation. The essential elements of a Rankine cycle system are: 1) a boiler to change liquid to vapor at high pressure; 2) a turbine to expand the vapor to derive mechanical energy; 3) a condenser to change low pressure exhaust vapor from the turbine to low pressure liquid; and 4) a pump to move condensate liquid back to the boiler at high pressure.

Various working fluids have been suggested for use in Rankine cycles, including HFC-245fa. However, there is a desire in the industry to provide a working fluid which is environmentally acceptable, has excellent thermodynamic properties, and can operate efficiently over a wide range of heat source temperatures.

### SUMMARY

The present invention includes heat transfer compositions for transferring heat and/or energy to and/or from an article, device or fluid, wherein said heat transfer composition comprises, consists essentially of, or consists of one or more compounds of Formula I:

F₅—S—C₂H_{X}—SF₅ Formula I
where x is 0, 2 or 4.

For the purposes of convenience, heat transfer compositions according to this paragraph are sometimes referred to herein as Composition 1.

The present invention includes heat transfer compositions for transferring heat and/or energy to and/or from an article, device or fluid, wherein said heat transfer composition comprises, consists essentially of or consists of a compound according to Formula I in which the carbon-carbon bond is a triple bond, in which case x is zero, as represented by Formula IA below:

F₅S—C≡C—F₅ Formula IA

For the purposes of convenience, heat transfer compositions according to this paragraph are sometimes referred to herein as Composition 2A.

The present invention includes heat transfer compositions for transferring heat and/or energy to and/or from an article, device or fluid, wherein said heat transfer composition comprises, consists essentially of, or consists of a compound according to Formula I in which the carbon-carbon bond is a double bond, in which case x is 2, as represented by Formula IB below:

F₅S—CH≡CH—SF₅ Formula IB

In this case, both cis and trans stereoisomers exist, and either or both isomers may be used. For the purposes of convenience, heat transfer compositions according to this paragraph are sometimes referred to herein as Composition 3B.

The present invention includes heat transfer compositions for transferring heat and/or energy to and/or from an article, device or fluid, wherein said heat transfer fluid comprises, consists essentially of, or consists of a compound according to Formula I in which the carbon-carbon bond is a double bond, in which case x is 2, and wherein the SF₅ groups are in the trans configuration, as represented by Formula 1B (trans) below:

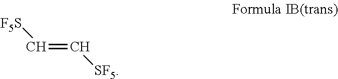

For the purposes of convenience, heat transfer compositions according to this paragraph are sometimes referred to herein as Composition 3Bt.

The present invention includes heat transfer compositions for transferring heat and/or energy to and/or from an article, device, or fluid, wherein said heat transfer fluid comprises, consists essentially of, or consists of a compound according to Formula I in which the carbon-carbon bond is a double bond, in which case x is 2, and wherein the SF₅ groups are in the cis configuration, as represented by Formula 1B (cis) below:

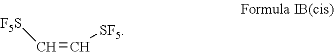

For the purposes of convenience, heat transfer compositions according to this paragraph are sometimes referred to herein as Composition 3Bc.

The present invention includes heat transfer compositions for transferring heat and/or energy to and/or from an article, device, or fluid, wherein said heat transfer fluid composition comprises, consists essentially of, or consists of a compound according to Formula I in which the carbon-carbon bond is a single bond, in which case x is 4, as represented by Formula IC below:

F₅S—CH₃—CH₂—SF₅ Formula IC

For the purposes of convenience, heat transfer compositions according to this paragraph are sometimes referred to herein as Composition 3C.

The present invention includes heat transfer compositions having a boiling point of from about 50 °C to about 250 °C and which comprise, consist essentially of or consist of one or more compounds according to Formula I. For the purposes of convenience, heat transfer compositions according to this paragraph are sometimes referred to herein as Composition 4A1.

The present invention includes heat transfer compositions having a boiling point of from about 50 °C to about 200 °C and which comprise, consist essentially of or consist of one or more compounds according to Formula I. For the purposes of convenience, heat transfer compositions according to this paragraph are sometimes referred to herein as Composition 4A2.

The present invention includes heat transfer compositions having a boiling point of from about 50 °C to about 150 °C and which comprise, consist essentially of or consist of one or more compounds according to Formula I. For the purposes of convenience, heat transfer compositions according to this paragraph are sometimes referred to herein as Composition 4A3.

The present invention includes heat transfer compositions having a boiling point of from about 50 °C to about 250 °C and which comprise, consist essentially of or consist of a compound of Formula 1A. For the purposes of convenience, heat transfer compositions according to this paragraph are sometimes referred to herein as Composition 4B1.

The present invention includes heat transfer compositions having a boiling point of from about 50 °C to about 200 °C and which comprise, consist essentially of or consist of a compound of Formula 1B. For the purposes of convenience, heat transfer compositions according to this paragraph are sometimes referred to herein as Composition 4B2.

The present invention includes heat transfer compositions having a boiling point of from about 50 °C to about 150 °C and which comprise, consist essentially of or consist of a compound of Formula 1B. For the purposes of convenience, heat transfer compositions according to this paragraph are sometimes referred to herein as Composition 4B3.

The present invention includes heat transfer compositions having a boiling point of from about 50 °C to about 250 °C and which comprise, consist essentially of or consist of a compound of Formula 1B (trans). For the purposes of convenience, heat transfer compositions according to this paragraph are sometimes referred to herein as Composition 4C1.

The present invention includes heat transfer compositions having a boiling point of from about 50 °C to about 200 °C and which comprise, consist essentially of or consist of a compound of Formula 1B (trans). For the purposes of convenience, heat transfer compositions according to this paragraph are sometimes referred to herein as Composition 4C2.

The present invention includes heat transfer compositions having a boiling point of from about 50 °C to about 150 °C and which comprise, consist essentially of or consist of a compound of Formula 1B (trans). For the purposes of convenience, heat transfer compositions according to this paragraph are sometimes referred to herein as Composition 4C3.

The present invention includes heat transfer compositions having a boiling point of from about 50 °C to about 250 °C and which comprise, consist essentially of or consist of a compound of Formula 1B (cis). For the purposes of convenience, heat transfer compositions according to this paragraph are sometimes referred to herein as Composition 4C4.

The present invention includes heat transfer compositions having a boiling point of from about 50 °C to about 200 °C and which comprise, consist essentially of or consist of a compound of Formula 1B (cis). For the purposes of convenience, heat transfer compositions according to this paragraph are sometimes referred to herein as Composition 4C5.

The present invention includes heat transfer compositions having a boiling point of from about 50 °C to about 150 °C and which comprise, consist essentially of or consist of a compound of Formula 1B (cis). For the purposes of convenience, heat transfer compositions according to this paragraph are sometimes referred to herein as Composition 4C6.

As used herein, “dielectric constant” is the value as measured in accordance with ASTM D150-11 at room temperature and 20 KHz.

The present invention includes heat transfer compositions having a boiling point of from about 50 °C to about 250 °C and a dielectric constant of less than about 5 and which comprise, consist essentially of or consist of one or more compounds according to Formula I. For the purposes of convenience, heat transfer compositions according to this paragraph are sometimes referred to herein as Composition 5A.

The present invention includes heat transfer compositions having a boiling point of from about 50 °C to about 250 °C and a dielectric constant of less than about 2.5 and which comprise, consist essentially of or consist of one or more compounds according to Formula I. For the purposes of convenience, heat transfer compositions according to this paragraph are sometimes referred to herein as Composition 5B.

The present invention includes heat transfer compositions having a boiling point of from about 50 °C to about 250 °C, a Global Warming Potential (GWP) of less than about 150 and a dielectric constant of less than about 2.5 and which comprise, consist essentially of or consist of one or more compounds according to Formula I. For the purposes of convenience, heat transfer compositions according to this paragraph are sometimes referred to herein as Composition 5C.

The present invention includes heat transfer compositions having a boiling point of from about 50 °C to about 250 °C, a Global Warming Potential (GWP) of less than about 75 and a dielectric constant of less than about 2.5 and which comprise, consist essentially of or consist of one or more compounds according to Formula I. For the purposes of convenience, heat transfer compositions according to this paragraph are sometimes referred to herein as Composition 5D.

The present invention includes methods for transferring heat and/or energy from an article, device or fluid comprising:
(a) providing the article, device or fluid; and
(b) transferring heat and/or energy between any composition of the present invention, including each of Compositions 1-5, and said article, device or fluid. For the purposes of convenience, methods according to this paragraph are sometimes referred to herein as Heat Transfer Methods 1.

As used herein, reference to a group of compositions, methods, and the like, defined by numbers, such as the reference in the preceding paragraph to “any of Compositions 1-5” specifically includes all such numbered compositions, including all numbered compositions with a suffix. For example, the reference to “any of Compositions 1-3” includes each of Composition 1, Composition 2A, Composition 3B, Composition 3Bt; Composition 3Bc; and Composition 3C.

The present invention includes systems and devices that include a heat transfer fluid for transferring heat within and/or to and/or from that system or device, said system and/or device comprising as the heat transfer fluid a composition according to any of Compositions 1-5. For the purposes of convenience, systems and/or devices according to this paragraph are sometimes referred to herein as System 1.

The present invention includes devices and systems for removing heat and/or energy from or adding heat and/or energy to an article, device or fluid comprising at least one vessel, container and/or conduit containing a thermal management composition (“TMC”) in thermal contact with said heat and/or energy, wherein said TMC is a TMC of the present invention, including each of Compositions 1-5. For the purposes of convenience, devices and systems according to this paragraph are sometimes referred to herein for convenience as System 2.

### BRIEF DESCRIPTION OF THE DRAWINGS

FIG. 1 is a schematic representation of a thermal management system of the present invention for use in electronics cooling.

FIG. 2A is a schematic representation of a first exemplary immersion cooling system according to the present invention for use in electronics cooling.

FIG. 2B is a schematic representation of a second exemplary immersion cooling system according to the present invention for use in electronics cooling.

FIGS. 3A and 3B are schematic illustrations of a battery thermal management system according to one embodiment of the present invention.

FIG. 4 is a semi-schematic diagram showing a portion of an exemplary datacenter cooling system.

FIG. 5 is a schematic diagram of a first exemplary organic Rankine cycle.

FIG. 6 is a schematic diagram of a second exemplary organic Rankine cycle.

FIG. 7 is a schematic diagram of a third exemplary organic Rankine cycle.

FIG. 8 is a schematic representation of a first exemplary high temperature heat pump system having an optional vapor injection system.

FIG. 9 is a schematic representation of a second exemplary high temperature heat pump system having an optional suction line liquid line heat exchanger.

FIG. 10 is a schematic representation of a third exemplary high temperature heat pump system.

FIG. 11 is a semi-schematic diagram of a battery cooling system.

FIG. 12 is a semi-schematic drawing of a heat pipe using a heat transfer composition of the present invention.

### DETAILED DESCRIPTION

### Definitions

As used herein, the following terms have the meanings indicated below unless specifically indicated otherwise.

The term heat transfer composition and related word forms, means a composition in the form of a fluid (liquid or gas) which is used to transfer heat or energy from one fluid, article or device to another location, and thus includes for example refrigerants, temperature control fluids and working fluids for Rankine cycles.

When a heat transfer composition is used in thermal management to keep a device or article within a particular temperature range (e.g., in electronic cooling), it is sometimes referred herein as a thermal management composition or TMC.

The component(s) that are present in a heat transfer composition for the purpose of transferring heat (as opposed to, for example, providing lubrication or stabilization) in a heat transfer system (e.g. a vapour compression heat transfer system), that component or combination of components are sometimes referred to herein as a refrigerant.

Device means an article, object or contrivance which is heated, cooled, or maintained at a predetermined temperature.

Operating Electronic Device, and related word forms, means a device, or a component of a device, which is in the process of performing its intended function by receiving, and/or transmitting and/or producing electrical energy and/or electronic signals. Thus, the term “operating electronic device” as used herein includes, for example, a battery which is in the process of providing a source of electrical energy to another component and also a battery which is being charged or recharged.

Thermal contact, and related forms thereof, includes direct contact with the surface and indirect contact though another body or fluid which facilitates the flow of heat between the surface and the fluid.

Global Warming Potential (“GWP”) was developed to allow comparisons of the global warming impact of different gases. It is a measure of how much energy the emission of one ton of a gas will absorb over a given period of time, relative to the emission of one ton of carbon dioxide. The larger GWP, the more that a given gas warms the Earth compared to CO₂ over that time period. The time period usually used for GWP is 100 years. GWP provides a common measure, which allows analysts to add up emission estimates of different gases. For the present application GWP refers to the value as determined by AR4.

Flammability as used in connection with this disclosure is a measure of whether a liquid (e.g., a pool of, primarily, a single component or a blend) is non-flammable, flammable, and/or exhibits a flash when an open flame is passed across its surface. This can only be determined with certainty experimentally by simulating a liquid spill by pouring the liquid of interest into a watch glass. The flammability of, primarily, a single component or a blend is characterized throughout the evaporation of the puddle to dryness. In blends with flammable components, the blend may be nonflammable initially, but may later exhibit a flash or become flammable due to blend composition shift during evaporation. The watch glass test is a conservative test because no external heat is applied to the watch glass, which is chilled by the evaporating solvent. The cold watch glass acts as a condenser for the vapors of higher boiling point blend components. In the case where a flammable component is high boiling, its concentration increases throughout the evaporation time, rendering the mixture more flammable than it would be under temperature-controlled blend segregation experiments.

Acceptable toxicity as used herein means a fluid that has toxicity within acceptable limits. This is experimentally determined by a toxicological screening study to assess in vivo acute oral toxicity at a dosage of 2,000 mg/kg/day and 4-hour acute inhalation toxicity at a concentration of about 20,000 ppm.

Capacity is the amount of cooling provided, in BTUs/hr, by the refrigerant in the refrigeration system. This is experimentally determined by multiplying the change in enthalpy in BTU/lb, of the refrigerant as it passes through the evaporator by the mass flow rate of the refrigerant. The enthalpy can be determined from the measurement of the pressure and temperature of the refrigerant. The capacity of the refrigeration system relates to the ability to maintain an area to be cooled at a specific temperature. The capacity of a refrigerant represents the amount of cooling or heating that it provides and provides some measure of the capability of a compressor to pump quantities of heat for a given volumetric flow rate of refrigerant. In other words, given a specific compressor, a refrigerant with a higher capacity will deliver more cooling or heating power.

Coefficient of Performance (hereinafter “COP”) is a universally accepted measure of refrigerant performance, especially useful in representing the relative thermodynamic efficiency of a refrigerant in a specific heating or cooling cycle involving evaporation or condensation of the refrigerant. In refrigeration engineering, this term expresses the ratio of useful refrigeration or cooling capacity to the energy applied by the compressor in compressing the vapor and therefore expresses the capability of a given compressor to pump quantities of heat for a given volumetric flow rate of a heat transfer fluid, such as a refrigerant. In other words, given a specific compressor, a refrigerant with a higher COP will deliver more cooling or heating power. One means for estimating COP of a refrigerant at specific operating conditions is from the thermodynamic properties of the refrigerant using standard refrigeration cycle analysis techniques (see for example, R.C. Downing, FLUOROCARBON REFRIGERANTS HANDBOOK, Chapter 3, Prentice-Hall, 1988 which is incorporated herein by reference in its entirety).

Thermal Efficiency is a measure of how efficiently one can convert energy from a heat source to work. This property is generally used to characterize the performance of an Organic Rankine Cycle System much like COP is used to measure the efficiency of a vapor compression system. One means for estimating COP of a refrigerant at specific operating conditions is from the thermodynamic properties of the refrigerant using standard refrigeration cycle analysis techniques (see for example, Engineering and Chemical Thermodynamics, Milo D. Koretsky. Wiley 2004, page 138.

As used herein, the singular forms “a”, “an”, and “the” include plural referents unless the content clearly dictates otherwise. As used in this specification, the term “or” is generally employed in its sense including “and/or” unless the content clearly dictates otherwise.

As used herein, the recitation of numerical ranges by endpoints includes all numbers subsumed within that range (e.g. 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

### Compounds

As indicated above, the present invention includes novel compounds according to Formula I:

F₅S—C₂H_{X}—SF₅Formula I
where x is 2 or 4.

Compounds according to the present invention can be formed by those skilled in the art based on the teaching contained herein, including specifically as disclosed in the Examples.

A novel compound in accordance with the present invention is:

F₅S—CH═CH—SF₅ Formula IB

Another novel compound in accordance with the present invention is:

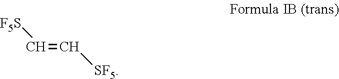

Another novel compound in accordance with the present invention is:

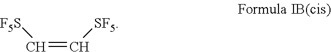

Another novel compound in accordance with the present invention is:

F₅S—CH₂CH₂—SF₅ Formula IC

The novel compounds of the present invention in accordance with Formula I are preferably used as refrigerants in heat transfer compositions (sometimes referred to as HTCs) and preferably have one or more of the following properties, and preferably at least two of the physical properties, as indicated in Table 1A below:

TABLE 1A
Preferred Properties of Formula I Compounds

| | |
|---|---|
| Property | FORMULA I PROPERTY RANGES |

| | | | |
|---|---|---|---|
| Boiling Point, | 50-250 | 50-200 | 50-150 |
| Dielectric | 1.5-10 | 1.5-5 | 1.5-3 |
| Dielectric | >30 | >40 | 30-80 |
| Thermal | >0.055 | >0.065 | 0.055-0.2 |

The preferred compounds of the present invention are sometimes referred to herein using the Compound No. designations as indicated below in Table 1B:

| Compound | Compound No. |
|---|---|
| F₅S—C≡C—SF₅ | 1 |
| Formula IA | |
| 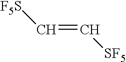 | 2Bt |
| Formula IB(trans) | |
| 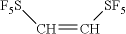 | 2Bc |
| Formula IB(cis) | |
| 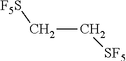 | 3C |
| Formula IC | |

In addition, the compounds of the present invention preferably have good miscibility with lubricants, including lubricants used in vapour compression refrigeration systems.

### Heat Transfer Compositions

The present invention provides various methods, processes and uses of the heat transfer compositions of the present invention, including each of Compositions 1-5, are fluids (i.e., liquids and/or gases) that may be used to transmit heat from one location to another (or from one body or article to another). For example, the heat transfer compositions may be used to keep the temperature of a device below a defined upper and/or above a defined lower temperature. In another example, the compositions may be used for heat and/or energy conversion, as in the capture of waste heat from industrial or other processes and the conversion to electrical or mechanical energy, in which case they may sometimes be referred to working fluids.

The heat transfer fluids of the present invention comprise a heat transfer composition of the present invention, including each of Compositions 1-5, wherein one or more Formula I compounds are present in the composition in an amount of at least about 50% by weight, or at least about 70% by weight, or at least about 90% by weight or at least about 95% by weight or at least about 99% by weight based on the heat transfer components, or the heat transfer fluid may consist essentially of or consist of one or more Formula I compounds.

The heat transfer fluids of the present invention comprise a heat transfer composition of the present invention, including each of Compositions 1-5, wherein the compound of Formula 1A is present in the composition in an amount of at least about 50% by weight, or at least about 70% by weight, or at least about 90% by weight or at least about 95% by weight or at least about 99% by weight based on heat transfer components, or the heat transfer fluid may consist essentially of or consist of the compound of Formula 1A.

The heat transfer fluids of the present invention comprise a heat transfer composition of the present invention, including each of Compositions 1-5, wherein compounds according to Formula 1B is present in the composition in an amount of at least about 50% by weight, or at least about 70% by weight, or at least about 90% by weight or at least about 95% by weight or at least about 99% by weight based on heat transfer components, or the heat transfer fluid may consist essentially of or consist of compounds according to Formula 1B.

The heat transfer fluids of the present invention comprise a heat transfer composition of the present invention, including each of Compositions 1-5, wherein the compound according to Formula 1B (trans) is present in the composition in an amount of at least about 50% by weight, or at least about 70% by weight, or at least about 90% by weight or at least about 95% by weight or at least about 99% by weight based on heat transfer components, or the heat transfer fluid may consist essentially of or consist of compounds according to Formula 1B (trans).

The heat transfer fluids of the present invention comprise a heat transfer composition of the present invention, including each of Compositions 1-5, wherein the compound according to Formula 1B (cis) is present in the composition in an amount of at least about 50% by weight, or at least about 70% by weight, or at least about 90% by weight or at least about 95% by weight or at least about 99% by weight based on heat transfer components, or the heat transfer fluid may consist essentially of or consist of compounds according to Formula 1B (cis). The heat transfer fluids of the present invention comprise a heat transfer composition of the present invention, including each of Compositions 1-5, wherein compounds according to Formula 1C is present in the composition in an amount of at least about 50% by weight, or at least about 70% by weight, or at least about 90% by weight or at least about 95% by weight or at least about 99% by weight, based on heat transfer components, or the heat transfer fluid may consist essentially of or consist of compounds according to Formula 1C.

The heat transfer compositions, the thermal management fluids, the working fluids and the refrigerants of the present invention, including each of Compositions 1-5, preferably has a low GWP, including a GWP of not greater than about 150, or not greater than about 75.

Preferably, the heat transfer composition of the present invention comprises a composition of the present invention, including each of Compositions 1-5, and a lubricant. The lubricant lubricates the refrigeration compressor using the refrigerant. The lubricant may be present in the heat transfer composition in amounts of from about 5% to about 30% by weight of heat transfer composition. Lubricants such as Polyol Esters (POEs), Poly Alkylene Glycols (PAGs), polyvinyl ethers (PVEs), poly(alpha-olefins) (PAOs), mineral oils, silicone oils, and alkyl benzenes, and combinations thereof may be used in the heat transfer compositions of the present invention. Non-limiting, exemplary lubricants are disclosed below.

### POE Lubricants

When used, the POE lubricant of the present invention includes in preferred embodiments comprises a neopentyl POE lubricant. As used herein, the term neopentyl POE lubricant refers to polyol esters (POEs) derived from a reaction between a neopentyl polyol (preferably pentaerythritol, trimethylolpropane, or neopentyl glycol, and in embodiments where higher viscosities are preferred, dipentaerythritol) and a linear or branched carboxylic acid.

Commercially available POEs include neopentyl glycol dipelargonate which is available as Emery 2917 (registered trademark) and Hatcol 2370 (registered trademark) and pentaerythritol derivatives including those sold under the trade designations Emkarate RL32-3MAF and Emkarate RL68H by CPI Fluid Engineering. Emkarate RL32-3MAF and Emkarate RL68H are preferred pentaerythritol derivative POE lubricants having the properties identified below:

| Property | RL32-3MAF | RL68H |
|---|---|---|
| Viscosity @ 40 °C (ASTM D467), cSt | about 31 | about 67 |
| Viscosity @ 100 °C (ASTM D467), cSt | about 5.6 | about 9.4 |
| Pour Point (ASTM D97), °C | about −40 | about −40 |

Other useful esters include phosphate esters, di-basic acid esters and fluoro esters.

### PVE Lubricants

The lubricant of the present invention can include PVE lubricants generally. In preferred embodiments the PVE lubricant is as PVE according to Formula II below:

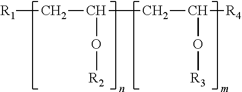

where R₂ and R₃ are each independently C1-C10 hydrocarbons, preferably C2-C8 hydrocarbons, and R₁ and R₄ are each independently alkyl, alkylene glycol, or polyoxyalkylene glycol units and n and m are selected preferably according to the needs of those skilled in the art to obtain a lubricant with the desired properties, and preferable n and m are selected to obtain a lubricant with a viscosity at 40 °C measured in accordance with ASTM D467 of from about 30 to about 70 cSt. Commercially available polyvinyl ethers include those lubricants sold under the trade designations FVC32D and FVC68D, from Idemitsu.

### Alkyl Benzene Lubricants

Commercially available alkyl benzene lubricants include Zerol 150 (registered trademark).

### Mineral Oils

Exemplary commercially available mineral oils include Witco LP 250 (registered trademark) from Witco, Zerol 300 (registered trademark) from Shrieve Chemical, Sunisco 3GS from Witco, and Calumet R015 from Calumet.

The heat transfer composition may include a compatibilizer for the purpose of aiding compatibility and/or solubility of the lubricant. Suitable compatibilizers may include propane, butanes, pentanes, and/or hexanes. When present, the compatibilizer is preferably present in an amount of from about 0.5% to about 5% by weight of the heat transfer composition. Surfactants and/or solubilizing agents may also be added to the present compositions to aid oil solubility, as disclosed by U.S. Pat. No. 6,516,837, the disclosure of which is incorporated by reference.

The present heat transfer compositions may include other components or compounds that supplement and/or enhance and/or contribute to the purpose of the composition. For convenience, such other components or compounds are referred to herein as co-agents generally, which may be in a particular case a co-heat transfer agent, a co-energy transfer agent, etc, as is specific to a particular application, method or system as discussed in detail hereinafter. The following Table 2 identifies heat transfer compositions (referred to as HTCs) that include compounds according to the present invention, including compounds of Formula I and Formula IB, and Compounds 1, 2Bt, 2Bc and 3C and optionally a co-agent in the amounts as indicated based on the total weight of the components in the “HTC No.” as identified in Column 1 of Table 2 below, with each amount being understood to be preceded by the word “about”:

TABLE 2
HEAT TRANSFER COMPOSITIONS

| | Wt. % Compound of the | |
|---|---|---|
| HTC No. | invention | Wt. % of Co-agent(s) |
| HTC1-1 | 100 Compound 1 | 0 |
| HTC1-2 | 1-99 Compound 1 | 1-99 |
| HTC1-3 | 1-10 Compound 1 | 90-99 |
| HTC1-4 | 30-50 Compound 1 | 50-70 |
| HTC1-5 | 50-70 Compound 1 | 30-50 |
| HTC1-6 | 70-90 Compound 1 | 10-30 |
| HTC1-7 | 90-99 Compound 1 | 1-10 |
| HTC2-1 | 100 Compound 2Bt | 0 |
| HTC2-2 | 1-99 Compound 2Bt | 1-99 |
| HTC2-3 | 1-10 Compound 2Bt | 90-99 |
| HTC2-4 | 30-50 Compound 2Bt | 50-70 |
| HTC2-5 | 50-70 Compound 2Bt | 30-50 |
| HTC2-6 | 70-90 Compound 2Bt | 10-30 |
| HTC2-7 | 90-99 Compound 2Bt | 1-10 |
| HTC3-1 | 100 Compound 2Bc | 0 |
| HTC3-2 | 1-99 Compound 2Bc | 1-99 |
| HTC3-3 | 1-10 Compound 2Bc | 90-99 |
| HTC3-4 | 30-50 Compound 2Bc | 50-70 |
| HTC3-5 | 50-70 Compound 2Bc | 30-50 |
| HTC3-6 | 70-90 Compound 2Bc | 10-30 |
| HTC3-7 | 90-99 Compound 2Bc | 1-10 |
| HTC4-1 | 100 Compound 3C | 0 |
| HTC4-2 | 1-99 Compound 3C | 1-99 |
| HTC4-3 | 1-10 Compound 3C | 90-99 |
| HTC4-4 | 30-50 Compound 3C | 50-70 |
| HTC4-5 | 50-70 Compound 3C | 30-50 |
| HTC4-6 | 70-90 Compound 3C | 10-30 |
| HTC4-7 | 90-99 Compound 3C | 1-10 |
| HTC5-B1 | 100 Formula IB | 0 |
| HTC5-B2 | 1-99 Formula IB | 1-99 |
| HTC5-B3 | 1-10 Formula IB | 90-99 |
| HTC5-B4 | 30-50 Formula IB | 50-70 |
| HTC5-B5 | 50-70 Formula IB | 30-50 |
| HTC5-B6 | 70-90 Formula IB | 10-30 |
| HTC5-B7 | 90-99 Formula IB | 1-10 |
| HTC6-1 | 100 Formula I | 0 |
| HTC6-2 | 1-99 Formula I | 1-99 |
| HTC6-3 | 1-10 Formula I | 90-99 |
| HTC6-4 | 30-50 Formula I | 50-70 |
| HTC6-5 | 50-70 Formula I | 30-50 |
| HTC6-6 | 70-90 Formula I | 10-30 |
| HTC6-7 | 90-99 Formula I | 1-10 |

### Uses, Methods, Systems and Devices

The present invention includes method for transferring heat as described herein, included methods as specifically described above and hereinafter.

The present invention also includes devices and systems for transferring heat as described herein, including devices and systems as specifically described above and hereinafter.

The heat transfer fluid, thermal management fluid, refrigerant, working fluid and heat transfer compositions, including each of Compositions 1-5 and each of HTC1-HTC6, of the invention are provided for use for heating and/or cooling as set out herein.

Thus, the present invention describes a method of heating or cooling a fluid or body using a heat transfer composition, thermal management fluid, refrigerant or working fluid of the invention, including each of Compositions 1-5 and each of HTC1-HTC6.

### Thermal Management Methods, Devices, Systems and Uses

In nearly every modern application of electronics, the dissipation of heat is an important consideration. For example, in portable and hand-held devices, the desire to miniaturize while adding functionality increases the thermal power density, which increases the challenge of cooling the electronics within them. As computational power increases within desktop computers, datacenters and telecommunications centers, so does the heat output. Power electronic devices such as the traction inverters in plug-in electric or hybrid vehicles, wind turbines, train engines, generators and various industrial processes make use of transistors that operate at ever higher currents and heat fluxes.

As discussed above, when a heat transfer fluid of the present invention, including each of Compositions 1-5 and each of HTC1-HTC6, is used in a method or device or system of cooling and/or heating in an electronic device, it is sometimes referred to herein as a thermal management fluid. The thermal management fluid therefore corresponds to the heat transfer fluid as discussed in this application.

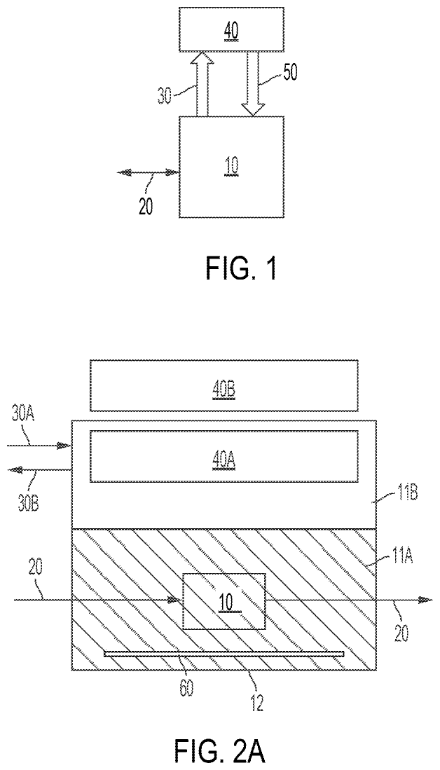

Preferred embodiments of the present thermal management methods, including Heat Transfer Methods 1, will now be described in connection with FIG. 1 in which an operating electronic device is shown schematically as 10 having a source of electrical energy and/or signals 20 flowing into and/or out of the device 10 and which generates heat as a result of its operation based on the electrical energy and/or signals 20. The thermal management fluid of the present invention is provided in thermal contact with the operating device 10 such that it removes heat, represented by the out flowing arrow 30. Heat is removed from the operating electronic device by sensible heat being added to the liquid thermal management fluid of the present invention (i.e., increasing the temperature of the liquid), or by causing a phase change in the thermal management liquid (i.e., vaporizing the liquid) or a combination of these. In preferred embodiments, the methods provide a supply of heat transfer fluid of the present invention, including each of Compositions 1-5, to the device 10 such that the flow of heat from the device 10 through the present heat transfer fluid 30 maintains the operating electrical device at or within a preferred operating temperature range. In preferred embodiments, the preferred operating temperature range of the electrical device is from about 70 °C to about 150 °C, and even more preferably from about 70 °C to about 120 °C, and the flow of heat 30 from the device 10 through the present heat transfer fluid energy maintains the operating electrical device at or within such preferred temperature ranges. Preferably, the heat transfer fluid 30 of the present invention, which has absorbed heat from the device, is in thermal contact with a heat sink, represented schematically as 40, at a temperature below the temperature of the heat transfer fluid 30 and thereby transfers the heat generated by the device 10 to the heat sink 40. In this way, the heat-depleted heat transfer fluid of the present invention 50 can be returned to the electronic device 10 to repeat the cycle of cooling.

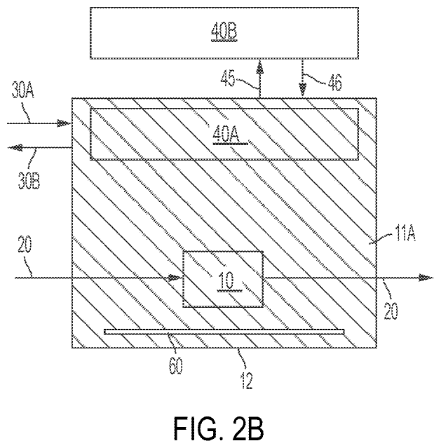

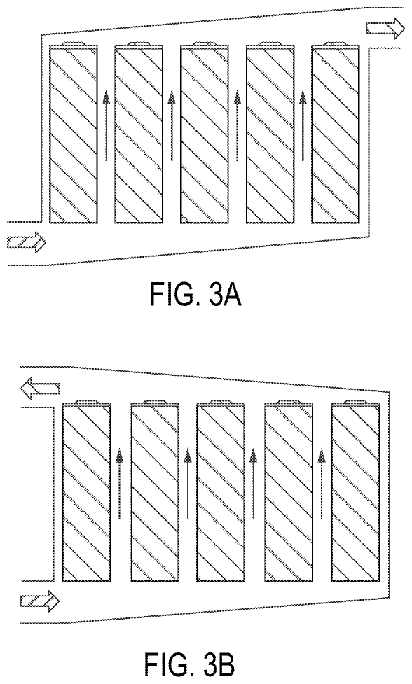

In a preferred embodiment of the present methods, the step of removing heat through a heat transfer composition of the present invention, including each of Compositions 1-5 and each of HTC1-HTC6, comprises evaporating the heat transfer composition of the present invention using the heat generated by the operation of the electronic device, and the step of transferring that heat from the heat transfer composition to the heat sink comprises condensing the heat transfer fluid by rejecting heat to the heat sink. In such methods, the temperature of the heat transfer fluid of the present invention, including each of Compositions 1-5 and each of HTC1-HTC6, during said evaporation step is preferably greater than 50 °C, or preferably greater than about 55 °C, or preferably in the range of from about 55 °C to about 85 °C, or preferably from about 65 °C to about 75 °C. Applicants have found that the present heat transfer fluids, including each of Compositions 1-5 and each of HTC1-HTC6, provide excellent performance in such methods and at the same time allow the use of relatively low cost, lightweight and reliable equipment to provide the necessary cooling, as will be explained further in connection with particular embodiments as described in connection with FIG. 2A below.

In a further preferred embodiment of the present methods, the step of removing heat through the present heat transfer composition, including each of Compositions 1-5 and each of HTC1-HTC6, comprises adding sensible heat to the liquid heat transfer composition of the present invention (e.g., raising the temperature of the liquid up to about 70 °C or less at about atmospheric pressure, i.e., wherein the fluid is not required to be in a high pressure container or vessel) using the heat generated by the operation of the electronic device, and the step of transferring that heat from the heat transfer composition to a heat sink and thereby reducing the liquid temperature by rejecting heat to the heat sink. The cooled liquid is then returned to thermal contact with the electrical device wherein the cycle starts over. In preferred embodiments, the temperature of the heat transfer liquid that is used to transfer heat to the heat sink is greater than about 40 °C, or preferably greater than about 55 °C, or preferably in the range of from about 45 °C to about 70 °C, or preferably from about 45 °C to about 65 °C, and preferably is at a pressure that is about atmospheric. Applicants have found that the present heat transfer liquids, including each of Compositions 1-5 and each of HTC1-HTC6, provide excellent performance in such methods and at the same time allow the use for relatively low cost, lightweight and reliable equipment to provide the necessary cooling, as will be explained further in connection with particular embodiments as described in connection with FIG. 2B below.

It will be appreciated by those skilled in the art that the present invention comprises systems and methods which use both sensible heat transfer and phase change heat transfer as described above.

A particular method according to the present invention will now be described in connection with FIGS. 2A and 2B in which an electronic device 10 is contained in an appropriate container 12, and preferably a sealed container, and is in direct contact with, and preferably fully immersed in liquid heat transfer composition of the present invention 11A (shown schematically by gray shading), including each of Compositions 1-5 and each of HTC1-HTC6. The operating electronic device 10 has a source of electrical energy and/or signals 20 flowing into and/or out of the container 12 and into and/or out of device 10, which generates heat as a result of its operation based on the electrical energy and/or signals 20. As those skilled in the art will appreciate, it is a significant challenge to discover a heat transfer fluid that can perform effectively in such applications since the fluid must not only provide all of the other properties mentioned above, it must be able to do so while in intimate contact with an operating electronic device, that is, one which involves the flow of electrical current/signals. It will be appreciated that many fluids that might be otherwise viable for use in such applications will not be useable because they will either short-out the device, degrade when exposed to the conditions created by the operation of the electronic device or have some other property detrimental to operation when in contact with an operating electronic device.

In contrast, the present methods produce excellent results by providing the thermal management fluid of the present invention, including each of Compositions 1-5 and each of HTC1-HTC6, in direct thermal and physical contact with the device 10 as it is operating. This heat of operation is safely and effectively transferred to the thermal management fluid 11A, including each of Compositions 1-5 and each of HTC1-HTC6, by: (a) causing the liquid phase of the fluid to evaporate and form vapor 11B; or (b) raising the temperature of the liquid thermal management fluid 11A; or (c) a combination of (a) and (b).

In the case of the phase change heat transfer systems of the present invention, reference is made herein to FIG. 2A. In such an operation, heat is carried away from the device 10 as the liquid evaporates and the vapor rises through the remaining thermal management liquid, including each of Compositions 1-5 and each of HTC1-HTC6, in the container 12. The thermal management fluid vapor 11B then rejects the heat it has absorbed to a heat sink 40, which can be an enclosed heat sink 40A and/or an external heat sink 40B. An example of a heat sink that is internal to the container 12 are condenser coils 30A and 30B with circulating liquid, such as water, at a temperature below the condensing temperature of the thermal management fluid vapor. An example of a heat sink that is external to the container 12 would be passing relatively cool ambient air over the container 12 (which preferably in such case include cooling fins or the like), which will serve to condense the heat transfer vapor 11B on the interior surface of the container. As a result of this condensation, liquid thermal management fluid is returned to the pool of liquid fluid 11A in which the device 10 remains immersed in operation.

In the case of a sensible heat transfer systems of the present invention, reference is made herein to FIG. 2B. In such an operation heat is carried away from the device as the temperature of liquid 11A, including each of Compositions 1-5 and each of HTC1-HTC6, increases upon accepting heat being generated by the device, which is immersed, and preferably substantially fully immersed in the thermal management fluid 11A of the present invention. The higher temperature thermal management fluid liquid 11A then rejects the heat it has absorbed to a heat sink 40, which can be an enclosed heat sink 40A and/or an external heat sink 40B. An example of a heat sink that is internal to the container 12 are cooling coils 30A and 30B with circulating liquid, such as water, at a temperature below the temperature of heated liquid. An example of a heat sink that is external to the container 12 would be removing heated liquid 11A from the container through a conduit 45 where it is thermally contacted with a cool fluid, such as might be provided by relatively cool ambient air or a refrigerant, which will serve to lower the temperature of the liquid. Cooled liquid is then returned via conduit 46.

Optionally, but preferably in certain embodiments involving thermal management of the batteries used in electronic vehicles, the thermal management system includes a heating element which is able to heat the thermal management fluid, including each of Compositions 1-5 and each of HTC1-HTC6, such as for example an electrical heating element 60 which is also immersed in the thermal management fluid. As those skilled in the art will appreciate, the batteries in electronic vehicles (which would correspond to the operating electronic device 10 in FIGS. 2A and 2B) can reach relatively low temperatures while parked outside in the winter months in many geographical locations, and frequently such low temperature conditions are not desirable for battery operation. Accordingly, the thermal management system of the present invention can include sensors and control modules (not shown) which turn on the heating element when the battery temperature is below a predetermined level. In such a case, the heater 60 would be activated, the thermal management liquid 11A would be heated, and would in turn transfer this heat to the electronic device 10 until the minimum temperature is reached. Thereafter during operation, the thermal management fluid of the present invention, including each of Compositions 1-5 and each of HTC1-HTC6, would serve the cooling function as described above.

For the purposes of this invention, the thermal management fluid, including each of Compositions 1-5 and each of HTC1-HTC6, can be in direct contact with the heat-generating component or in indirect contact with the heat-generating component.

When the thermal management fluid, including each of Compositions 1-5 and each of HTC1-HTC6, is in indirect contact with the heat-generating component, the thermal management fluid can be used in a closed system in the electronic device, which may include at least two heat exchangers. When the thermal management fluid is used to cool the heat-generating component, heat can be transferred from the component to the thermal management fluid, usually through a heat exchanger in contact with at least a part of the component or the heat can be transferred to circulating air which can conduct the heat to a heat exchanger that is in thermal contact with the thermal management fluid.

In a particularly preferred feature of the present invention, the thermal management fluid, including each of Compositions 1-5 and each of HTC1-HTC6, is in direct contact with the heat-generating component. In particular, the heat generating component is fully or partially immersed in the thermal management fluid. Preferably the heat generating component is fully immersed in the thermal management fluid. The thermal management fluid, as a warmed fluid or as a vapor, can then be circulated to a heat exchanger which takes the heat from the fluid or vapor and transfers it to the outside environment. After this heat transfer, the cooled thermal management fluid (cooled or condensed) is recycled back into the system to cool the heat-generating component.

When the thermal management fluid is a single-phase liquid, it will remain liquid when heated by the heat-generating component. Thus, the thermal management fluid can be brought into contact with the heat generating component, resulting in the removal of the heat from the heat generating component and the production of a thermal management fluid with a higher temperature. The thermal management fluid is then transported to a secondary cooling loop, such as a radiator or another refrigerated system. An example of such a system is illustrated in FIG. 2, where the thermal management fluid enters a battery pack enclosure containing a number of cells and exits the enclosure having taken up heat from the battery pack.

When the thermal management fluid of the present invention, including each of Compositions 1-5 and each of HTC1-HTC6, is present in two phases, the heat-generating component is in thermal contact with the thermal management fluid and transfers heat to the thermal management fluid, resulting in the boiling of the thermal management fluid. The thermal management fluid is then condensed. An example of such a system is where the heat-generating component is immersed in the thermal management fluid and an external cooling circuit condenses the boiling fluid into a liquid state.

Electrical conductivity and/or dielectric strength of a thermal management fluid becomes important if the fluid comes in direct contact with the electronic components of the electronic device (such as in direct immersion cooling), or if the thermal management fluid leaks out of a cooling loop or is spilled during maintenance and comes in contact with the electrical circuits. Thus, the thermal management fluid of the present invention, including each of Compositions 1-5 and each of HTC1-HTC6, is preferably an electrically insulating thermal management fluid.

The thermal management fluid of the present invention, including each of Compositions 1-5 and each of HTC1-HTC6, may be recirculated passively or actively in the device, for example by using mechanical equipment such as a pump. In a preferred feature of the present invention, the thermal management fluid of the present invention, including each of Compositions 1-5 and each of HTC1-HTC6, is recirculated passively in the device.

Passive recirculating systems work by transferring heat from the heat-generating component to the thermal management fluid until it typically is vaporized, allowing the heated vapor to proceed to a heat exchange surface at which it transfers its heat to the heat exchanger surface and condenses back into a liquid. It will be appreciated that the heat exchange surface can be part of a separate heat exchange unit and/or can be integral with the container, as described above for example in connection with FIG. 2. The condensed liquid then returns, preferably fully passively by the force of gravity and/or a wicking structure, into the thermal management fluid in contact with the heat-generating component. Thus, in a preferred feature of the invention, the step of transferring heat from the heat-generating component to the thermal management fluid of the present invention, including each of Compositions 1-5 and each of HTC1-HTC6, causes the thermal management fluid to vaporize.

Examples of passive recirculating systems include a heat pipe or a thermosyphon. Such systems passively recirculate the thermal management fluid of the present invention, including each of Compositions 1-5 and each of HTC1-HTC6, using gravity. In such a system, the thermal management fluid is heated by the heat-generating component, resulting in a heated thermal management fluid which is less dense and more buoyant. This thermal management fluid travels to a storage container, such as a tank where it cools and condenses. The cooled thermal management fluid then flows back to the heat source.

The present invention includes use of the present compounds, including each of Compositions 1-5 and each of HTC1-HTC6, to cool and optionally heat electronic devices that produce or include a component that is a heat-generating component. The electronic device includes a heat-generating component. The heat-generating component can be any component that includes an electronic element that generates heat as part of its operation. For the purposes of this invention, the heat generating component includes but is not limited to: semiconductor integrated circuits (ICs), electrochemical cells, power transistors, resistors, and electroluminescent elements, such as microprocessors, wafers used to manufacture semiconductor devices, power control semiconductors, electrical distribution switch gear, power transformers, circuit boards, multi-chip modules, packaged or unpackaged semiconductor devices, semiconductor integrated circuits, fuel cells, lasers (conventional or laser diodes), light emitting diodes (LEDs), and electrochemical cells, e.g. used for high power applications such as, for example, hybrid or electric vehicles.

For the purpose of this invention, the electronic device includes but is not limited to: personal computers, microprocessors, servers, cell phones, tablets, digital home appliances (e.g. televisions, media players, games consoles etc.), personal digital assistants, Datacenters, batteries both stationary and in vehicles, hybrid or electric vehicles, wind turbine, train engine, or generator. Preferably the electronic device is a hybrid or electric vehicle.

The present invention further relates to an electronic device comprising a thermal management fluid of the invention, including each of Compositions 1-5 and each of HTC1-HTC6. For the purposes of this invention, the thermal management fluid is provided for cooling and/or heating the electronic device.

The present invention further relates to an electronic device comprising a heat generating component and a thermal management fluid of the invention, including each of Compositions 1-5 and each of HTC1-HTC6. For the purposes of this invention, the electronic device can further comprise a heat exchanger, particularly where the heat exchanger is in contact with at least a part of the heat generating component.

The present invention further relates to an electronic device comprising a heat generating component, a heat exchanger, a pump and a thermal management fluid of the invention, including each of Compositions 1-5 and each of HTC1-HTC6.

For the purposes of this invention, the heat generating component can be selected from semiconductor integrated circuits (ICs), electrochemical cells, power transistors, resistors, and electroluminescent elements, such as microprocessors, wafers used to manufacture semiconductor devices, power control semiconductors, electrical distribution switch gear, power transformers, circuit boards, multi-chip modules, packaged or unpackaged semiconductor devices, semiconductor integrated circuits, fuel cells, lasers (conventional or laser diodes), light emitting diodes (LEDs), and electrochemical cells, e.g. used for high power applications such as, for example, hybrid or electric vehicles.

For the purpose of this invention, the electronic device can be selected from personal computers, microprocessors, servers, cell phones, tablets, digital home appliances (e.g. televisions, media players, games consoles etc.), personal digital assistants, Datacenters, hybrid or electric vehicles, batteries both stationary and in vehicles, wind turbine, train engine, or generator, preferably wherein the electronic device is a hybrid or electric vehicle.

The invention further relates to the use of a thermal management fluid of the invention, including each of Compositions 1-5 and each of HTC1-HTC6, for cooling an electronic device. For the purpose of this invention, the electronic device can be selected from personal computers, microprocessors, servers, cell phones, tablets, digital home appliances (e.g. televisions, media players, games consoles etc.), personal digital assistants, Datacenters, hybrid or electric vehicles, batteries both stationary and in vehicles, wind turbine, train engine, or generator, preferably wherein the electronic device is a hybrid or electric vehicle.

Examples of the present thermal management methods useful for battery cooling, including lithium-ion battery cooling, including Heat Transfer Methods 1, will now be described in connection with FIG. 11. A vehicle battery pack with a self-contained liquid cooling system 10 comprising a module 12 formed of a container 14 with an interior space 16 for supporting a battery assembly 18. The container 14 is a closed and sealed container 14 for forming a self-contained liquid cooling system 10. The battery assembly 18 includes a plurality of battery cells 20 such as a plurality of Lithium-ion (Li-ion) batteries for use in a hybrid vehicle. In another embodiment, the plurality of battery cells 20 is Li-ion batteries for use in a Battery Electric Vehicle (BEV). Additional batteries for use with other prime mover vehicles may be provided with the liquid cooling system 10 of the present invention, where each battery cell includes active material for generating power from an electrochemical reaction within the interior space 16 of the container 14. The battery cells 20 are preferably stacked to form a battery cell stack 22. In the embodiment shown, a gap 24 between each battery cell 20 is between 0.25-0.50 mm, forming a fluid channel 26 between each battery cell 20. In another embodiment, the gap 24 may be less than 0.25 mm. It is understood that other gap sizes can be used as desired.

A composition of the present invention, including each of Compositions 1-5 and each of HTC1-HTC6, is disposed within the interior space 16 of the container 14 and the fluid level shown is such that the battery assembly 18 is completely immersed within the composition of the present invention. The composition of the present invention, including each of including each of Compositions 1-5 and each of HTC1-HTC6, is in contact with the battery cells 20 through the fluid channels 26 formed by gaps 24.

A heating element 34 is located at a base area 36 of the container 14. The heating element 34 shown is an electronic heating element. It is understood that other heating element types may be used. The heating element 34 is shown as a single element; however, multiple heating elements 34 such as heating plates may be provided.

A cooling element 38 is located at an upper area 40 of the container 14. The cooling element 38 may be a chilled water condenser having an inlet 42 and an outlet 44 extending beyond the walls of the sealed container 14 for importing and exporting water for the cooling element 38. In another embodiment, the cooling element 38 may be a chilled water plate. In still another embodiment, the cooling element 38 may be a thin aluminum heat sink having external chilled water travelling through the cooling element 38. The cooling element 38 may be a graphite foil impregnated with an electrically nonconductive polymer. The cooling element may also be formed from copper.

In the embodiment shown, arrows “A” and “B” indicate a flow 28 of the composition of the present invention, including each of including each of Compositions 1-5 and each of HTC1-HTC6. Upon heating of each battery cell 20 by the heating element 34, the coolant 28 of the present invention, including each of including each of Compositions 1-5 and each of HTC1-HTC6, is exposed to a front surface area 30 and a rear surface area 32 of the battery cells 20, and will boil. The heated coolant 28 will rise and flow to the top of the battery cell stack 22 to be cooled by the cooling element 38. The cooled coolant 28 will return to the base area 36, generally following either coolant paths “A” or “B.” Where the general location of the coolant 28 at the moment of boiling is located within the fluid channels 26 of the battery cells 20 in the center area and toward a side 50 of the container 14, the coolant 28 will tend to follow flow path “A”. Similarly, if the general location of the dielectric coolant 28 at the moment of boiling is located within the fluid channels 26 of the battery cells 20 in the center area and toward an opposing side 52 of the container 14, the dielectric coolant 28 will tend to follow flow path “B”.

A coolant temperature sensor 46 is located on or near the cooling element 38. In the embodiment shown, the temperature sensor 46 is located within the area of the outlet 44 of the cooling element 38 and measures a temperature of the dielectric coolant 28 of the present invention at a point of exposure to the cooling element. The temperature sensor 46 may be located anywhere within the battery cell stack 22 as desired.

A coolant level sensor 48 is also provided and is located near the upper area 40 of the container 14 to measure the fluid level of the dielectric coolant 28 within the container 14, ensuring complete immersion of the battery assembly 18 within the dielectric coolant 28.

While the above description of cooling by immersion in a composition of the present invention, including each of Compositions 1-5 and each of HTC1-HTC6 is made in connection with cooling of a batter, the same basic procedure, and all variations thereof within the skill of the art, can be used to cool any electronic component or device, as described herein, including each of those devices identified in Table 3 below.

The following Table 3 identifies preferred electronic devices and components that are cooled according to immersion cooling systems and methods or the present invention (with reference to NR indicating that there is no requirement associated with that feature and the reference to Comp No. being to the Composition Nos. as defined above, and WF being to the Working Fluid defined by number in Table 2 above):

TABLE 3
PREFERRED IMMERSION COOLING
SYSTEMS AND METHODS

| Composition No. | Device/Electronic | Located | Operating |
|---|---|---|---|
| or HTC No. | Component | in | Temp, °C |
| Composition 1 | Battery | NR | NR |
| Composition 2A | Battery | NR | NR |
| Composition 3B | Battery | NR | NR |
| Composition 3Bt | Battery | NR | NR |
| Composition 3Bc | Battery | NR | NR |
| Composition 3C | Battery | NR | NR |
| Composition 4A1 | Battery | NR | NR |
| Composition 4A2 | Battery | NR | NR |
| Composition 4A3 | Battery | NR | NR |
| Composition 4B1 | Battery | NR | NR |
| Composition 4B2 | Battery | NR | NR |
| Composition 4B3 | Battery | NR | NR |
| Composition 4C1 | Battery | NR | NR |
| Composition 4C2 | Battery | NR | NR |
| Composition 4C3 | Battery | NR | NR |
| Composition 5A | Battery | NR | NR |
| Composition 5B | Battery | NR | NR |
| Composition 5C | Battery | NR | NR |
| Composition 5D | Battery | NR | NR |
| HTC1-1 | Battery | NR | NR |
| HTC1-2 | Battery | NR | NR |
| HTC1-3 | Battery | NR | NR |
| HTC1-4 | Battery | NR | NR |
| HTC1-5 | Battery | NR | NR |
| HTC1-6 | Battery | NR | NR |
| HTC1-7 | Battery | NR | NR |
| HTC2-1 | Battery | NR | NR |
| HTC2-2 | Battery | NR | NR |
| HTC2-3 | Battery | NR | NR |
| HTC2-4 | Battery | NR | NR |
| HTC2-5 | Battery | NR | NR |
| HTC2-6 | Battery | NR | NR |
| HTC2-7 | Battery | NR | NR |
| HTC3-1 | Battery | NR | NR |
| HTC3-2 | Battery | NR | NR |
| HTC3-3 | Battery | NR | NR |
| HTC3-4 | Battery | NR | NR |
| HTC3-5 | Battery | NR | NR |
| HTC3-6 | Battery | NR | NR |
| HTC3-7 | Battery | NR | NR |
| HTC4-1 | Battery | NR | NR |
| HTC4-2 | Battery | NR | NR |
| HTC4-3 | Battery | NR | NR |
| HTC4-4 | Battery | NR | NR |
| HTC4-5 | Battery | NR | NR |
| HTC4-6 | Battery | NR | NR |
| HTC4-7 | Battery | NR | NR |
| HTC5-B1 | Battery | NR | NR |
| HTC5-B2 | Battery | NR | NR |
| HTC5-B3 | Battery | NR | NR |
| HTC5-B4 | Battery | NR | NR |
| HTC5-B5 | Battery | NR | NR |
| HTC5-B6 | Battery | NR | NR |
| HTC5-B7 | Battery | NR | NR |
| HTC6-C1 | Battery | NR | NR |
| HTC6-2 | Battery | NR | NR |
| HTC6-3 | Battery | NR | NR |
| HTC6-4 | Battery | NR | NR |
| HTC6-5 | Battery | NR | NR |
| HTC6-6 | Battery | NR | NR |
| HTC6-7 | Battery | NR | NR |
| Composition 1 | Battery | NR | 50-150 |
| Composition 2A | Battery | NR | 50-150 |
| Composition 3B | Battery | NR | 50-150 |
| Composition 3Bt | Battery | NR | 50-150 |
| Composition 3Bc | Battery | NR | 50-150 |
| Composition 3C | Battery | NR | 50-150 |
| Composition 4A1 | Battery | NR | 50-150 |
| Composition 4A2 | Battery | NR | 50-150 |
| Composition 4A3 | Battery | NR | 50-150 |
| Composition 4B1 | Battery | NR | 50-150 |
| Composition 4B2 | Battery | NR | 50-150 |
| Composition 4B3 | Battery | NR | 50-150 |
| Composition 4C1 | Battery | NR | 50-150 |
| Composition 4C2 | Battery | NR | 50-150 |
| Composition 4C3 | Battery | NR | 50-150 |
| Composition 5A | Battery | NR | 50-150 |
| Composition 5B | Battery | NR | 50-150 |
| Composition 5C | Battery | NR | 50-150 |
| Composition 5D | Battery | NR | 50-150 |
| HTC1-1 | Battery | NR | 50-150 |
| HTC1-2 | Battery | NR | 50-150 |
| HTC1-3 | Battery | NR | 50-150 |
| HTC1-4 | Battery | NR | 50-150 |
| HTC1-5 | Battery | NR | 50-150 |
| HTC1-6 | Battery | NR | 50-150 |
| HTC1-7 | Battery | NR | 50-150 |
| HTC2-1 | Battery | NR | 50-150 |
| HTC2-2 | Battery | NR | 50-150 |
| HTC2-3 | Battery | NR | 50-150 |
| HTC2-4 | Battery | NR | 50-150 |
| HTC2-5 | Battery | NR | 50-150 |
| HTC2-6 | Battery | NR | 50-150 |
| HTC2-7 | Battery | NR | 50-150 |
| HTC3-1 | Battery | NR | 50-150 |
| HTC3-2 | Battery | NR | 50-150 |
| HTC3-3 | Battery | NR | 50-150 |
| HTC3-4 | Battery | NR | 50-150 |
| HTC3-5 | Battery | NR | 50-150 |
| HTC3-6 | Battery | NR | 50-150 |
| HTC3-7 | Battery | NR | 50-150 |
| HTC4-1 | Battery | NR | 50-150 |
| HTC4-2 | Battery | NR | 50-150 |
| HTC4-3 | Battery | NR | 50-150 |
| HTC4-4 | Battery | NR | 50-150 |
| HTC4-5 | Battery | NR | 50-150 |
| HTC4-6 | Battery | NR | 50-150 |
| HTC4-7 | Battery | NR | 50-150 |
| HTC4-1 | Battery | NR | 50-150 |
| HTC5-B1 | Battery | NR | 50-150 |
| HTC5-B2 | Battery | NR | 50-150 |
| HTC5-B3 | Battery | NR | 50-150 |
| HTC5-B4 | Battery | NR | 50-150 |
| HTC5-B5 | Battery | NR | 50-150 |
| HTC5-B6 | Battery | NR | 50-150 |
| HTC5-B7 | Battery | NR | 50-150 |
| HTC6-1 | Battery | NR | 50-150 |
| HTC6-2 | Battery | NR | 50-150 |
| HTC6-3 | Battery | NR | 50-150 |
| HTC6-4 | Battery | NR | 50-150 |
| HTC6-5 | Battery | NR | 50-150 |
| HTC6-6 | Battery | NR | 50-150 |
| HTC6-7 | Battery | NR | 50-150 |
| Composition 1 | Battery | NR | 70-120 |
| Composition 2A | Battery | NR | 70-120 |
| Composition 3B | Battery | NR | 70-120 |
| Composition 3Bt | Battery | NR | 70-120 |
| Composition 3Bc | Battery | NR | 70-120 |
| Composition 3C | Battery | NR | 70-120 |
| Composition 4A1 | Battery | NR | 70-120 |
| Composition 4A2 | Battery | NR | 70-120 |
| Composition 4A3 | Battery | NR | 70-120 |
| Composition 4B1 | Battery | NR | 70-120 |
| Composition 4B2 | Battery | NR | 70-120 |
| Composition 4B3 | Battery | NR | 70-120 |
| Composition 4C1 | Battery | NR | 70-120 |
| Composition 4C2 | Battery | NR | 70-120 |
| Composition 4C3 | Battery | NR | 70-120 |
| Composition 5A | Battery | NR | 70-120 |
| Composition 5B | Battery | NR | 70-120 |
| Composition 5C | Battery | NR | 70-120 |
| Composition 5D | Battery | NR | 70-120 |
| HTC1-1 | Battery | NR | 70-120 |
| HTC1-2 | Battery | NR | 70-120 |
| HTC1-3 | Battery | NR | 70-120 |
| HTC1-4 | Battery | NR | 70-120 |
| HTC1-5 | Battery | NR | 70-120 |
| HTC1-6 | Battery | NR | 70-120 |
| HTC1-7 | Battery | NR | 70-120 |
| HTC2-1 | Battery | NR | 70-120 |
| HTC2-2 | Battery | NR | 70-120 |
| HTC2-3 | Battery | NR | 70-120 |
| HTC2-4 | Battery | NR | 70-120 |
| HTC2-5 | Battery | NR | 70-120 |
| HTC2-6 | Battery | NR | 70-120 |
| HTC2-7 | Battery | NR | 70-120 |
| HTC3-1 | Battery | NR | 70-120 |
| HTC3-2 | Battery | NR | 70-120 |
| HTC3-3 | Battery | NR | 70-120 |
| HTC3-4 | Battery | NR | 70-120 |
| HTC3-5 | Battery | NR | 70-120 |
| HTC3-6 | Battery | NR | 70-120 |
| HTC4-1 | Battery | NR | 70-120 |
| HTC4-2 | Battery | NR | 70-120 |
| HTC4-3 | Battery | NR | 70-120 |
| HTC4-4 | Battery | NR | 70-120 |
| HTC4-5 | Battery | NR | 70-120 |
| HTC4-6 | Battery | NR | 70-120 |
| HTC4-7 | Battery | NR | 70-120 |
| HTC4-1 | Battery | NR | 70-120 |
| HTC5-B1 | Battery | NR | 70-120 |
| HTC5-B2 | Battery | NR | 70-120 |
| HTC5-B3 | Battery | NR | 70-120 |
| HTC5-B4 | Battery | NR | 70-120 |
| HTC5-B5 | Battery | NR | 70-120 |
| HTC5-B6 | Battery | NR | 70-120 |
| HTC5-B7 | Battery | NR | 70-120 |
| HTC6-1 | Battery | NR | 70-120 |
| HTC6-2 | Battery | NR | 70-120 |
| HTC6-3 | Battery | NR | 70-120 |
| HTC6-4 | Battery | NR | 70-120 |
| HTC6-5 | Battery | NR | 70-120 |
| HTC6-6 | Battery | NR | 70-120 |
| HTC6-7 | Battery | NR | 70-120 |
| Composition 1 | Battery | Electric Vehicle | NR |
| Composition 2A | Battery | Electric Vehicle | NR |
| Composition 3B | Battery | Electric Vehicle | NR |
| Composition 3Bt | Battery | Electric Vehicle | NR |
| Composition 3Bc | Battery | Electric Vehicle | NR |
| Composition 3C | Battery | Electric Vehicle | NR |
| Composition 4A1 | Battery | Electric Vehicle | NR |
| Composition 4A2 | Battery | Electric Vehicle | NR |
| Composition 4A3 | Battery | Electric Vehicle | NR |
| Composition 4B1 | Battery | Electric Vehicle | NR |
| Composition 4B2 | Battery | Electric Vehicle | NR |
| Composition 4B3 | Battery | Electric Vehicle | NR |
| Composition 4C1 | Battery | Electric Vehicle | NR |
| Composition 4C2 | Battery | Electric Vehicle | NR |
| Composition 4C3 | Battery | Electric Vehicle | NR |
| Composition 5A | Battery | Electric Vehicle | NR |
| Composition 5B | Battery | Electric Vehicle | NR |
| Composition 5C | Battery | Electric Vehicle | NR |
| Composition 5D | Battery | Electric Vehicle | NR |
| HTC1-1 | Battery | Electric Vehicle | NR |
| HTC1-2 | Battery | Electric Vehicle | NR |
| HTC1-3 | Battery | Electric Vehicle | NR |
| HTC1-4 | Battery | Electric Vehicle | NR |
| HTC1-5 | Battery | Electric Vehicle | NR |
| HTC1-6 | Battery | Electric Vehicle | NR |
| HTC1-7 | Battery | Electric Vehicle | NR |
| HTC2-1 | Battery | Electric Vehicle | NR |
| HTC2-2 | Battery | Electric Vehicle | NR |
| HTC2-3 | Battery | Electric Vehicle | NR |
| HTC2-4 | Battery | Electric Vehicle | NR |
| HTC2-5 | Battery | Electric Vehicle | NR |
| HTC2-6 | Battery | Electric Vehicle | NR |
| HTC2-7 | Battery | Electric Vehicle | NR |
| HTC3-1 | Battery | Electric Vehicle | NR |
| HTC3-2 | Battery | Electric Vehicle | NR |
| HTC3-3 | Battery | Electric Vehicle | NR |
| HTC3-4 | Battery | Electric Vehicle | NR |
| HTC3-5 | Battery | Electric Vehicle | NR |
| HTC3-6 | Battery | Electric Vehicle | NR |
| HTC3-7 | Battery | Electric Vehicle | NR |
| HTC4-1 | Battery | Electric Vehicle | NR |
| HTC4-2 | Battery | Electric Vehicle | NR |
| HTC4-3 | Battery | Electric Vehicle | NR |
| HTC4-4 | Battery | Electric Vehicle | NR |
| HTC4-5 | Battery | Electric Vehicle | NR |
| HTC4-6 | Battery | Electric Vehicle | NR |
| HTC4-7 | Battery | Electric Vehicle | NR |
| HTC4-1 | Battery | Electric Vehicle | NR |
| HTC5-B1 | Battery | Electric Vehicle | NR |
| HTC5-B2 | Battery | Electric Vehicle | NR |
| HTC5-B3 | Battery | Electric Vehicle | NR |
| HTC5-B4 | Battery | Electric Vehicle | NR |
| HTC5-B5 | Battery | Electric Vehicle | NR |
| HTC5-B6 | Battery | Electric Vehicle | NR |
| HTC5-B7 | Battery | Electric Vehicle | NR |
| HTC6-1 | Battery | Electric Vehicle | NR |
| HTC6-2 | Battery | Electric Vehicle | NR |
| HTC6-3 | Battery | Electric Vehicle | NR |
| HTC6-4 | Battery | Electric Vehicle | NR |
| HTC6-5 | Battery | Electric Vehicle | NR |
| HTC6-6 | Battery | Electric Vehicle | NR |
| HTC6-7 | Battery | Electric Vehicle | NR |
| Composition 1 | Battery | Electric Vehicle | 50-150 |
| Composition 2A | Battery | Electric Vehicle | 50-150 |
| Composition 3B | Battery | Electric Vehicle | 50-150 |
| Composition 3Bt | Battery | Electric Vehicle | 50-150 |
| Composition 3Bc | Battery | Electric Vehicle | 50-150 |
| Composition 3C | Battery | Electric Vehicle | 50-150 |
| Composition 4A1 | Battery | Electric Vehicle | 50-150 |
| Composition 4A2 | Battery | Electric Vehicle | 50-150 |
| Composition 4A3 | Battery | Electric Vehicle | 50-150 |
| Composition 4B1 | Battery | Electric Vehicle | 50-150 |
| Composition 4B2 | Battery | Electric Vehicle | 50-150 |
| Composition 4B3 | Battery | Electric Vehicle | 50-150 |
| Composition 4C1 | Battery | Electric Vehicle | 50-150 |
| Composition 4C2 | Battery | Electric Vehicle | 50-150 |
| Composition 4C3 | Battery | Electric Vehicle | 50-150 |
| Composition 5A | Battery | Electric Vehicle | 50-150 |
| Composition 5B | Battery | Electric Vehicle | 50-150 |
| Composition 5C | Battery | Electric Vehicle | 50-150 |
| Composition 5D | Battery | Electric Vehicle | 50-150 |
| HTC1-1 | Battery | Electric Vehicle | 50-150 |
| HTC1-2 | Battery | Electric Vehicle | 50-150 |
| HTC1-3 | Battery | Electric Vehicle | 50-150 |
| HTC1-4 | Battery | Electric Vehicle | 50-150 |
| HTC1-5 | Battery | Electric Vehicle | 50-150 |
| HTC1-6 | Battery | Electric Vehicle | 50-150 |
| HTC1-7 | Battery | Electric Vehicle | 50-150 |
| HTC2-1 | Battery | Electric Vehicle | 50-150 |
| HTC2-2 | Battery | Electric Vehicle | 50-150 |
| HTC2-3 | Battery | Electric Vehicle | 50-150 |
| HTC2-4 | Battery | Electric Vehicle | 50-150 |
| HTC2-5 | Battery | Electric Vehicle | 50-150 |
| HTC2-6 | Battery | Electric Vehicle | 50-150 |
| HTC2-7 | Battery | Electric Vehicle | 50-150 |
| HTC3-1 | Battery | Electric Vehicle | 50-150 |
| HTC3-2 | Battery | Electric Vehicle | 50-150 |
| HTC3-3 | Battery | Electric Vehicle | 50-150 |
| HTC3-4 | Battery | Electric Vehicle | 50-150 |
| HTC3-5 | Battery | Electric Vehicle | 50-150 |
| HTC3-6 | Battery | Electric Vehicle | 50-150 |
| HTC3-7 | Battery | Electric Vehicle | 50-150 |
| HTC4-1 | Battery | Electric Vehicle | 50-150 |
| HTC4-2 | Battery | Electric Vehicle | 50-150 |
| HTC4-3 | Battery | Electric Vehicle | 50-150 |
| HTC4-4 | Battery | Electric Vehicle | 50-150 |
| HTC4-5 | Battery | Electric Vehicle | 50-150 |
| HTC4-6 | Battery | Electric Vehicle | 50-150 |
| HTC4-7 | Battery | Electric Vehicle | 50-150 |
| HTC4-1 | Battery | Electric Vehicle | 50-150 |
| HTC5-B1 | Battery | Electric Vehicle | 50-150 |
| HTC5-B2 | Battery | Electric Vehicle | 50-150 |
| HTC5-B3 | Battery | Electric Vehicle | 50-150 |
| HTC5-B4 | Battery | Electric Vehicle | 50-150 |
| HTC5-B5 | Battery | Electric Vehicle | 50-150 |
| HTC5-B6 | Battery | Electric Vehicle | 50-150 |
| HTC5-B7 | Battery | Electric Vehicle | 50-150 |
| HTC6-1 | Battery | Electric Vehicle | 50-150 |
| HTC6-2 | Battery | Electric Vehicle | 50-150 |
| HTC6-3 | Battery | Electric Vehicle | 50-150 |
| HTC6-4 | Battery | Electric Vehicle | 50-150 |
| HTC6-5 | Battery | Electric Vehicle | 50-150 |
| HTC6-6 | Battery | Electric Vehicle | 50-150 |
| HTC6-7 | Battery | Electric Vehicle | 50-150 |
| Composition 1 | Battery | Electric Vehicle | 70-120 |
| Composition 2A | Battery | Electric Vehicle | 70-120 |
| Composition 3B | Battery | Electric Vehicle | 70-120 |
| Composition 3Bt | Battery | Electric Vehicle | 70-120 |
| Composition 3Bc | Battery | Electric Vehicle | 70-120 |
| Composition 3C | Battery | Electric Vehicle | 70-120 |
| Composition 4A1 | Battery | Electric Vehicle | 70-120 |
| Composition 4A2 | Battery | Electric Vehicle | 70-120 |
| Composition 4A3 | Battery | Electric Vehicle | 70-120 |
| Composition 4B1 | Battery | Electric Vehicle | 70-120 |
| Composition 4B2 | Battery | Electric Vehicle | 70-120 |
| Composition 4B3 | Battery | Electric Vehicle | 70-120 |
| Composition 4C1 | Battery | Electric Vehicle | 70-120 |
| Composition 4C2 | Battery | Electric Vehicle | 70-120 |
| Composition 4C3 | Battery | Electric Vehicle | 70-120 |
| Composition 5A | Battery | Electric Vehicle | 70-120 |
| Composition 5B | Battery | Electric Vehicle | 70-120 |
| Composition 5C | Battery | Electric Vehicle | 70-120 |
| Composition 5D | Battery | Electric Vehicle | 70-120 |
| HTC1-1 | Battery | Electric Vehicle | 70-120 |
| HTC1-2 | Battery | Electric Vehicle | 70-120 |
| HTC1-3 | Battery | Electric Vehicle | 70-120 |
| HTC1-4 | Battery | Electric Vehicle | 70-120 |
| HTC1-5 | Battery | Electric Vehicle | 70-120 |
| HTC1-6 | Battery | Electric Vehicle | 70-120 |
| HTC1-7 | Battery | Electric Vehicle | 70-120 |
| HTC2-1 | Battery | Electric Vehicle | 70-120 |
| HTC2-2 | Battery | Electric Vehicle | 70-120 |
| HTC2-3 | Battery | Electric Vehicle | 70-120 |
| HTC2-4 | Battery | Electric Vehicle | 70-120 |
| HTC2-5 | Battery | Electric Vehicle | 70-120 |
| HTC2-6 | Battery | Electric Vehicle | 70-120 |
| HTC2-7 | Battery | Electric Vehicle | 70-120 |
| HTC3-1 | Battery | Electric Vehicle | 70-120 |
| HTC3-2 | Battery | Electric Vehicle | 70-120 |
| HTC3-3 | Battery | Electric Vehicle | 70-120 |
| HTC3-4 | Battery | Electric Vehicle | 70-120 |
| HTC3-5 | Battery | Electric Vehicle | 70-120 |
| HTC3-6 | Battery | Electric Vehicle | 70-120 |
| HTC3-7 | Battery | Electric Vehicle | 70-120 |
| HTC4-1 | Battery | Electric Vehicle | 70-120 |
| HTC4-2 | Battery | Electric Vehicle | 70-120 |
| HTC4-3 | Battery | Electric Vehicle | 70-120 |
| HTC4-4 | Battery | Electric Vehicle | 70-120 |
| HTC4-5 | Battery | Electric Vehicle | 70-120 |
| HTC4-6 | Battery | Electric Vehicle | 70-120 |
| HTC4-7 | Battery | Electric Vehicle | 70-120 |
| HTC4-1 | Battery | Electric Vehicle | 70-120 |
| HTC5-B1 | Battery | Electric Vehicle | 70-120 |
| HTC5-B2 | Battery | Electric Vehicle | 70-120 |
| HTC5-B3 | Battery | Electric Vehicle | 70-120 |
| HTC5-B4 | Battery | Electric Vehicle | 70-120 |
| HTC5-B5 | Battery | Electric Vehicle | 70-120 |
| HTC5-B6 | Battery | Electric Vehicle | 70-120 |
| HTC5-B7 | Battery | Electric Vehicle | 70-120 |
| HTC6-1 | Battery | Electric Vehicle | 70-120 |
| HTC6-2 | Battery | Electric Vehicle | 70-120 |
| HTC6-3 | Battery | Electric Vehicle | 70-120 |
| HTC6-4 | Battery | Electric Vehicle | 70-120 |
| HTC6-5 | Battery | Electric Vehicle | 70-120 |
| HTC6-6 | Battery | Electric Vehicle | 70-120 |
| HTC6-7 | Battery | Electric Vehicle | 70-120 |
| Composition 1 | Battery | Hybrid Vehicle | NR |
| Composition 2A | Battery | Hybrid Vehicle | NR |
| Composition 3B | Battery | Hybrid Vehicle | NR |
| Composition 3Bt | Battery | Hybrid Vehicle | NR |
| Composition 3Bc | Battery | Hybrid Vehicle | NR |
| Composition 3C | Battery | Hybrid Vehicle | NR |
| Composition 4A1 | Battery | Hybrid Vehicle | NR |
| Composition 4A2 | Battery | Hybrid Vehicle | NR |
| Composition 4A3 | Battery | Hybrid Vehicle | NR |
| Composition 4B1 | Battery | Hybrid Vehicle | NR |
| Composition 4B2 | Battery | Hybrid Vehicle | NR |
| Composition 4B3 | Battery | Hybrid Vehicle | NR |
| Composition 4C1 | Battery | Hybrid Vehicle | NR |
| Composition 4C2 | Battery | Hybrid Vehicle | NR |
| Composition 4C3 | Battery | Hybrid Vehicle | NR |
| Composition 5A | Battery | Hybrid Vehicle | NR |
| Composition 5B | Battery | Hybrid Vehicle | NR |
| Composition 5C | Battery | Hybrid Vehicle | NR |
| Composition 5D | Battery | Hybrid Vehicle | NR |
| HTC1-1 | Battery | Hybrid Vehicle | NR |
| HTC1-2 | Battery | Hybrid Vehicle | NR |
| HTC1-3 | Battery | Hybrid Vehicle | NR |
| HTC1-4 | Battery | Hybrid Vehicle | NR |
| HTC1-5 | Battery | Hybrid Vehicle | NR |
| HTC1-6 | Battery | Hybrid Vehicle | NR |
| HTC1-7 | Battery | Hybrid Vehicle | NR |
| HTC2-1 | Battery | Hybrid Vehicle | NR |
| HTC2-2 | Battery | Hybrid Vehicle | NR |
| HTC2-3 | Battery | Hybrid Vehicle | NR |
| HTC2-4 | Battery | Hybrid Vehicle | NR |
| HTC2-5 | Battery | Hybrid Vehicle | NR |
| HTC2-6 | Battery | Hybrid Vehicle | NR |
| HTC2-7 | Battery | Hybrid Vehicle | NR |
| HTC3-1 | Battery | Hybrid Vehicle | NR |
| HTC3-2 | Battery | Hybrid Vehicle | NR |
| HTC3-3 | Battery | Hybrid Vehicle | NR |
| HTC3-4 | Battery | Hybrid Vehicle | NR |
| HTC3-5 | Battery | Hybrid Vehicle | NR |
| HTC3-6 | Battery | Hybrid Vehicle | NR |
| HTC3-7 | Battery | Hybrid Vehicle | NR |
| HTC4-1 | Battery | Hybrid Vehicle | NR |
| HTC4-2 | Battery | Hybrid Vehicle | NR |
| HTC4-3 | Battery | Hybrid Vehicle | NR |
| HTC4-4 | Battery | Hybrid Vehicle | NR |
| HTC4-5 | Battery | Hybrid Vehicle | NR |
| HTC4-6 | Battery | Hybrid Vehicle | NR |
| HTC4-7 | Battery | Hybrid Vehicle | NR |
| HTC4-1 | Battery | Hybrid Vehicle | NR |
| HTC5-B1 | Battery | Hybrid Vehicle | NR |
| HTC5-B2 | Battery | Hybrid Vehicle | NR |
| HTC5-B3 | Battery | Hybrid Vehicle | NR |
| HTC5-B4 | Battery | Hybrid Vehicle | NR |
| HTC5-B5 | Battery | Hybrid Vehicle | NR |
| HTC5-B6 | Battery | Hybrid Vehicle | NR |
| HTC5-B7 | Battery | Hybrid Vehicle | NR |
| HTC6-1 | Battery | Hybrid Vehicle | NR |
| HTC6-2 | Battery | Hybrid Vehicle | NR |
| HTC6-3 | Battery | Hybrid Vehicle | NR |
| HTC6-4 | Battery | Hybrid Vehicle | NR |
| HTC6-5 | Battery | Hybrid Vehicle | NR |
| HTC6-6 | Battery | Hybrid Vehicle | NR |
| HTC6-7 | Battery | Hybrid Vehicle | NR |
| Composition 1 | Battery | Hybrid Vehicle | 50-150 |
| Composition 2A | Battery | Hybrid Vehicle | 50-150 |
| Composition 3B | Battery | Hybrid Vehicle | 50-150 |
| Composition 3Bt | Battery | Hybrid Vehicle | 50-150 |
| Composition 3Bc | Battery | Hybrid Vehicle | 50-150 |
| Composition 3C | Battery | Hybrid Vehicle | 50-150 |
| Composition 4A1 | Battery | Hybrid Vehicle | 50-150 |
| Composition 4A2 | Battery | Hybrid Vehicle | 50-150 |
| Composition 4A3 | Battery | Hybrid Vehicle | 50-150 |
| Composition 4B1 | Battery | Hybrid Vehicle | 50-150 |
| Composition 4B2 | Battery | Hybrid Vehicle | 50-150 |
| Composition 4B3 | Battery | Hybrid Vehicle | 50-150 |
| Composition 4C1 | Battery | Hybrid Vehicle | 50-150 |
| Composition 4C2 | Battery | Hybrid Vehicle | 50-150 |
| Composition 4C3 | Battery | Hybrid Vehicle | 50-150 |
| Composition 5A | Battery | Hybrid Vehicle | 50-150 |
| Composition 5B | Battery | Hybrid Vehicle | 50-150 |
| Composition 5C | Battery | Hybrid Vehicle | 50-150 |
| Composition 5D | Battery | Hybrid Vehicle | 50-150 |
| HTC1-1 | Battery | Hybrid Vehicle | 50-150 |
| HTC1-2 | Battery | Hybrid Vehicle | 50-150 |
| HTC1-3 | Battery | Hybrid Vehicle | 50-150 |
| HTC1-4 | Battery | Hybrid Vehicle | 50-150 |
| HTC1-5 | Battery | Hybrid Vehicle | 50-150 |
| HTC1-6 | Battery | Hybrid Vehicle | 50-150 |
| HTC1-7 | Battery | Hybrid Vehicle | 50-150 |
| HTC2-1 | Battery | Hybrid Vehicle | 50-150 |
| HTC2-2 | Battery | Hybrid Vehicle | 50-150 |
| HTC2-3 | Battery | Hybrid Vehicle | 50-150 |
| HTC2-4 | Battery | Hybrid Vehicle | 50-150 |
| HTC2-5 | Battery | Hybrid Vehicle | 50-150 |
| HTC2-6 | Battery | Hybrid Vehicle | 50-150 |
| HTC2-7 | Battery | Hybrid Vehicle | 50-150 |
| HTC3-1 | Battery | Hybrid Vehicle | 50-150 |
| HTC3-2 | Battery | Hybrid Vehicle | 50-150 |
| HTC3-3 | Battery | Hybrid Vehicle | 50-150 |
| HTC3-4 | Battery | Hybrid Vehicle | 50-150 |
| HTC3-5 | Battery | Hybrid Vehicle | 50-150 |
| HTC3-6 | Battery | Hybrid Vehicle | 50-150 |
| HTC3-7 | Battery | Hybrid Vehicle | 50-150 |
| HTC4-1 | Battery | Hybrid Vehicle | 50-150 |
| HTC4-2 | Battery | Hybrid Vehicle | 50-150 |
| HTC4-3 | Battery | Hybrid Vehicle | 50-150 |
| HTC4-4 | Battery | Hybrid Vehicle | 50-150 |
| HTC4-5 | Battery | Hybrid Vehicle | 50-150 |
| HTC4-6 | Battery | Hybrid Vehicle | 50-150 |
| HTC4-7 | Battery | Hybrid Vehicle | 50-150 |
| HTC4-1 | Battery | Hybrid Vehicle | 50-150 |
| HTC5-B1 | Battery | Hybrid Vehicle | 50-150 |
| HTC5-B2 | Battery | Hybrid Vehicle | 50-150 |
| HTC5-B3 | Battery | Hybrid Vehicle | 50-150 |
| HTC5-B4 | Battery | Hybrid Vehicle | 50-150 |
| HTC5-B5 | Battery | Hybrid Vehicle | 50-150 |
| HTC5-B6 | Battery | Hybrid Vehicle | 50-150 |
| HTC5-B7 | Battery | Hybrid Vehicle | 50-150 |
| HTC6-1 | Battery | Hybrid Vehicle | 50-150 |
| HTC6-2 | Battery | Hybrid Vehicle | 50-150 |
| HTC6-3 | Battery | Hybrid Vehicle | 50-150 |
| HTC6-4 | Battery | Hybrid Vehicle | 50-150 |
| HTC6-5 | Battery | Hybrid Vehicle | 50-150 |
| HTC6-6 | Battery | Hybrid Vehicle | 50-150 |
| HTC6-7 | Battery | Hybrid Vehicle | 50-150 |
| Composition 1 | Battery | Hybrid Vehicle | 70-120 |
| Composition 2A | Battery | Hybrid Vehicle | 70-120 |
| Composition 3B | Battery | Hybrid Vehicle | 70-120 |
| Composition 3Bt | Battery | Hybrid Vehicle | 70-120 |
| Composition 3Bc | Battery | Hybrid Vehicle | 70-120 |
| Composition 3C | Battery | Hybrid Vehicle | 70-120 |
| Composition 4A1 | Battery | Hybrid Vehicle | 70-120 |
| Composition 4A2 | Battery | Hybrid Vehicle | 70-120 |
| Composition 4A3 | Battery | Hybrid Vehicle | 70-120 |
| Composition 4B1 | Battery | Hybrid Vehicle | 70-120 |
| Composition 4B2 | Battery | Hybrid Vehicle | 70-120 |
| Composition 4B3 | Battery | Hybrid Vehicle | 70-120 |
| Composition 4C1 | Battery | Hybrid Vehicle | 70-120 |
| Composition 4C2 | Battery | Hybrid Vehicle | 70-120 |
| Composition 4C3 | Battery | Hybrid Vehicle | 70-120 |
| Composition 5A | Battery | Hybrid Vehicle | 70-120 |
| Composition 5B | Battery | Hybrid Vehicle | 70-120 |
| Composition 5C | Battery | Hybrid Vehicle | 70-120 |
| Composition 5D | Battery | Hybrid Vehicle | 70-120 |
| HTC1-1 | Battery | Hybrid Vehicle | 70-120 |
| HTC1-2 | Battery | Hybrid Vehicle | 70-120 |
| HTC1-3 | Battery | Hybrid Vehicle | 70-120 |
| HTC1-4 | Battery | Hybrid Vehicle | 70-120 |
| HTC1-5 | Battery | Hybrid Vehicle | 70-120 |
| HTC1-6 | Battery | Hybrid Vehicle | 70-120 |
| HTC1-7 | Battery | Hybrid Vehicle | 70-120 |
| HTC2-1 | Battery | Hybrid Vehicle | 70-120 |
| HTC2-2 | Battery | Hybrid Vehicle | 70-120 |
| HTC2-3 | Battery | Hybrid Vehicle | 70-120 |
| HTC2-4 | Battery | Hybrid Vehicle | 70-120 |
| HTC2-5 | Battery | Hybrid Vehicle | 70-120 |
| HTC2-6 | Battery | Hybrid Vehicle | 70-120 |
| HTC2-7 | Battery | Hybrid Vehicle | 70-120 |
| HTC3-1 | Battery | Hybrid Vehicle | 70-120 |
| HTC3-2 | Battery | Hybrid Vehicle | 70-120 |
| HTC3-3 | Battery | Hybrid Vehicle | 70-120 |
| HTC3-4 | Battery | Hybrid Vehicle | 70-120 |
| HTC3-5 | Battery | Hybrid Vehicle | 70-120 |
| HTC3-6 | Battery | Hybrid Vehicle | 70-120 |
| HTC3-7 | Battery | Hybrid Vehicle | 70-120 |
| HTC4-1 | Battery | Hybrid Vehicle | 70-120 |
| HTC4-2 | Battery | Hybrid Vehicle | 70-120 |
| HTC4-3 | Battery | Hybrid Vehicle | 70-120 |
| HTC4-4 | Battery | Hybrid Vehicle | 70-120 |
| HTC4-5 | Battery | Hybrid Vehicle | 70-120 |
| HTC4-6 | Battery | Hybrid Vehicle | 70-120 |
| HTC4-7 | Battery | Hybrid Vehicle | 70-120 |
| HTC4-1 | Battery | Hybrid Vehicle | 70-120 |
| HTC5-B1 | Battery | Hybrid Vehicle | 70-120 |
| HTC5-B2 | Battery | Hybrid Vehicle | 70-120 |
| HTC5-B3 | Battery | Hybrid Vehicle | 70-120 |
| HTC5-B4 | Battery | Hybrid Vehicle | 70-120 |
| HTC5-B5 | Battery | Hybrid Vehicle | 70-120 |
| HTC5-B6 | Battery | Hybrid Vehicle | 70-120 |
| HTC5-B7 | Battery | Hybrid Vehicle | 70-120 |
| HTC6-1 | Battery | Hybrid Vehicle | 70-120 |
| HTC6-2 | Battery | Hybrid Vehicle | 70-120 |
| HTC6-3 | Battery | Hybrid Vehicle | 70-120 |
| HTC6-4 | Battery | Hybrid Vehicle | 70-120 |
| HTC6-5 | Battery | Hybrid Vehicle | 70-120 |
| HTC6-6 | Battery | Hybrid Vehicle | 70-120 |
| HTC6-7 | Battery | Hybrid Vehicle | 70-120 |
| Composition 1 | Electric Drive Motor | Electric Vehicle | NR |
| Composition 2A | Electric Drive Motor | Electric Vehicle | NR |
| Composition 3B | Electric Drive Motor | Electric Vehicle | NR |
| Composition 3Bt | Electric Drive Motor | Electric Vehicle | NR |
| Composition 3Bc | Electric Drive Motor | Electric Vehicle | NR |
| Composition 3C | Electric Drive Motor | Electric Vehicle | NR |
| Composition 4A1 | Electric Drive Motor | Electric Vehicle | NR |
| Composition 4A2 | Electric Drive Motor | Electric Vehicle | NR |
| Composition 4A3 | Electric Drive Motor | Electric Vehicle | NR |
| Composition 4B1 | Electric Drive Motor | Electric Vehicle | NR |
| Composition 4B2 | Electric Drive Motor | Electric Vehicle | NR |
| Composition 4B3 | Electric Drive Motor | Electric Vehicle | NR |
| Composition 4C1 | Electric Drive Motor | Electric Vehicle | NR |
| Composition 4C2 | Electric Drive Motor | Electric Vehicle | NR |
| Composition 4C3 | Electric Drive Motor | Electric Vehicle | NR |
| Composition 5A | Electric Drive Motor | Electric Vehicle | NR |
| Composition 5B | Electric Drive Motor | Electric Vehicle | NR |
| Composition 5C | Electric Drive Motor | Electric Vehicle | NR |
| Composition 5D | Electric Drive Motor | Electric Vehicle | NR |
| HTC1-1 | Electric Drive Motor | Electric Vehicle | NR |
| HTC1-2 | Electric Drive Motor | Electric Vehicle | NR |
| HTC1-3 | Electric Drive Motor | Electric Vehicle | NR |
| HTC1-4 | Electric Drive Motor | Electric Vehicle | NR |
| HTC1-5 | Electric Drive Motor | Electric Vehicle | NR |
| HTC1-6 | Electric Drive Motor | Electric Vehicle | NR |
| HTC1-7 | Electric Drive Motor | Electric Vehicle | NR |
| HTC2-1 | Electric Drive Motor | Electric Vehicle | NR |
| HTC2-2 | Electric Drive Motor | Electric Vehicle | NR |
| HTC2-3 | Electric Drive Motor | Electric Vehicle | NR |
| HTC2-4 | Electric Drive Motor | Electric Vehicle | NR |
| HTC2-5 | Electric Drive Motor | Electric Vehicle | NR |
| HTC2-6 | Electric Drive Motor | Electric Vehicle | NR |
| HTC2-7 | Electric Drive Motor | Electric Vehicle | NR |
| HTC3-1 | Electric Drive Motor | Electric Vehicle | NR |
| HTC3-2 | Electric Drive Motor | Electric Vehicle | NR |
| HTC3-3 | Electric Drive Motor | Electric Vehicle | NR |
| HTC3-4 | Electric Drive Motor | Electric Vehicle | NR |
| HTC3-5 | Electric Drive Motor | Electric Vehicle | NR |
| HTC3-6 | Electric Drive Motor | Electric Vehicle | NR |
| HTC3-7 | Electric Drive Motor | Electric Vehicle | NR |
| HTC4-1 | Electric Drive Motor | Electric Vehicle | NR |
| HTC4-2 | Electric Drive Motor | Electric Vehicle | NR |
| HTC4-3 | Electric Drive Motor | Electric Vehicle | NR |
| HTC4-4 | Electric Drive Motor | Electric Vehicle | NR |
| HTC4-5 | Electric Drive Motor | Electric Vehicle | NR |
| HTC4-6 | Electric Drive Motor | Electric Vehicle | NR |
| HTC4-7 | Electric Drive Motor | Electric Vehicle | NR |
| HTC4-1 | Electric Drive Motor | Electric Vehicle | NR |
| HTC5-B1 | Electric Drive Motor | Electric Vehicle | NR |
| HTC5-B2 | Electric Drive Motor | Electric Vehicle | NR |
| HTC5-B3 | Electric Drive Motor | Electric Vehicle | NR |
| HTC5-B4 | Electric Drive Motor | Electric Vehicle | NR |
| HTC5-B5 | Electric Drive Motor | Electric Vehicle | NR |
| HTC5-B6 | Electric Drive Motor | Electric Vehicle | NR |
| HTC5-B7 | Electric Drive Motor | Electric Vehicle | NR |
| HTC6-1 | Electric Drive Motor | Electric Vehicle | NR |
| HTC6-2 | Electric Drive Motor | Electric Vehicle | NR |
| HTC6-3 | Electric Drive Motor | Electric Vehicle | NR |
| HTC6-4 | Electric Drive Motor | Electric Vehicle | NR |
| HTC6-5 | Electric Drive Motor | Electric Vehicle | NR |
| HTC6-6 | Electric Drive Motor | Electric Vehicle | NR |
| HTC6-7 | Electric Drive Motor | Electric Vehicle | NR |
| Composition 1 | Electric Drive Motor | Hybrid Vehicle | NR |
| Composition 2A | Electric Drive Motor | Hybrid Vehicle | NR |
| Composition 3B | Electric Drive Motor | Hybrid Vehicle | NR |
| Composition 3Bt | Electric Drive Motor | Hybrid Vehicle | NR |
| Composition 3Bc | Electric Drive Motor | Hybrid Vehicle | NR |
| Composition 3C | Electric Drive Motor | Hybrid Vehicle | NR |
| Composition 4A1 | Electric Drive Motor | Hybrid Vehicle | NR |
| Composition 4A2 | Electric Drive Motor | Hybrid Vehicle | NR |
| Composition 4A3 | Electric Drive Motor | Hybrid Vehicle | NR |
| Composition 4B1 | Electric Drive Motor | Hybrid Vehicle | NR |
| Composition 4B2 | Electric Drive Motor | Hybrid Vehicle | NR |
| Composition 4B3 | Electric Drive Motor | Hybrid Vehicle | NR |
| Composition 4C1 | Electric Drive Motor | Hybrid Vehicle | NR |
| Composition 4C2 | Electric Drive Motor | Hybrid Vehicle | NR |
| Composition 4C3 | Electric Drive Motor | Hybrid Vehicle | NR |
| Composition 5A | Electric Drive Motor | Hybrid Vehicle | NR |
| Composition 5B | Electric Drive Motor | Hybrid Vehicle | NR |
| Composition 5C | Electric Drive Motor | Hybrid Vehicle | NR |
| Composition 5D | Electric Drive Motor | Hybrid Vehicle | NR |
| HTC1-1 | Electric Drive Motor | Hybrid Vehicle | NR |
| HTC1-2 | Electric Drive Motor | Hybrid Vehicle | NR |
| HTC1-3 | Electric Drive Motor | Hybrid Vehicle | NR |
| HTC1-4 | Electric Drive Motor | Hybrid Vehicle | NR |
| HTC1-5 | Electric Drive Motor | Hybrid Vehicle | NR |
| HTC1-6 | Electric Drive Motor | Hybrid Vehicle | NR |
| HTC1-7 | Electric Drive Motor | Hybrid Vehicle | NR |
| HTC2-1 | Electric Drive Motor | Hybrid Vehicle | NR |
| HTC2-2 | Electric Drive Motor | Hybrid Vehicle | NR |
| HTC2-3 | Electric Drive Motor | Hybrid Vehicle | NR |
| HTC2-4 | Electric Drive Motor | Hybrid Vehicle | NR |
| HTC2-5 | Electric Drive Motor | Hybrid Vehicle | NR |
| HTC2-6 | Electric Drive Motor | Hybrid Vehicle | NR |
| HTC2-7 | Electric Drive Motor | Hybrid Vehicle | NR |
| HTC3-1 | Electric Drive Motor | Hybrid Vehicle | NR |
| HTC3-2 | Electric Drive Motor | Hybrid Vehicle | NR |
| HTC3-3 | Electric Drive Motor | Hybrid Vehicle | NR |
| HTC3-4 | Electric Drive Motor | Hybrid Vehicle | NR |
| HTC3-5 | Electric Drive Motor | Hybrid Vehicle | NR |
| HTC3-6 | Electric Drive Motor | Hybrid Vehicle | NR |
| HTC3-7 | Electric Drive Motor | Hybrid Vehicle | NR |
| HTC4-1 | Electric Drive Motor | Hybrid Vehicle | NR |
| HTC4-2 | Electric Drive Motor | Hybrid Vehicle | NR |
| HTC4-3 | Electric Drive Motor | Hybrid Vehicle | NR |
| HTC4-4 | Electric Drive Motor | Hybrid Vehicle | NR |
| HTC4-5 | Electric Drive Motor | Hybrid Vehicle | NR |
| HTC4-6 | Electric Drive Motor | Hybrid Vehicle | NR |
| HTC4-7 | Electric Drive Motor | Hybrid Vehicle | NR |
| HTC4-1 | Electric Drive Motor | Hybrid Vehicle | NR |
| HTC5-B1 | Electric Drive Motor | Hybrid Vehicle | NR |
| HTC5-B2 | Electric Drive Motor | Hybrid Vehicle | NR |
| HTC5-B3 | Electric Drive Motor | Hybrid Vehicle | NR |
| HTC5-B4 | Electric Drive Motor | Hybrid Vehicle | NR |
| HTC5-B5 | Electric Drive Motor | Hybrid Vehicle | NR |
| HTC5-B6 | Electric Drive Motor | Hybrid Vehicle | NR |
| HTC5-B7 | Electric Drive Motor | Hybrid Vehicle | NR |
| HTC6-1 | Electric Drive Motor | Hybrid Vehicle | NR |
| HTC6-2 | Electric Drive Motor | Hybrid Vehicle | NR |
| HTC6-3 | Electric Drive Motor | Hybrid Vehicle | NR |
| HTC6-4 | Electric Drive Motor | Hybrid Vehicle | NR |
| HTC6-5 | Electric Drive Motor | Hybrid Vehicle | NR |
| HTC6-6 | Electric Drive Motor | Hybrid Vehicle | NR |
| HTC6-7 | Electric Drive Motor | Hybrid Vehicle | NR |
| Composition 1 | PCB | NR | NR |
| Composition 2A | PCB | NR | NR |
| Composition 3B | PCB | NR | NR |
| Composition 3Bt | PCB | NR | NR |
| Composition 3Bc | PCB | NR | NR |
| Composition 3C | PCB | NR | NR |
| Composition 4A1 | PCB | NR | NR |
| Composition 4A2 | PCB | NR | NR |
| Composition 4A3 | PCB | NR | NR |
| Composition 4B1 | PCB | NR | NR |
| Composition 4B2 | PCB | NR | NR |
| Composition 4B3 | PCB | NR | NR |
| Composition 4C1 | PCB | NR | NR |
| Composition 4C2 | PCB | NR | NR |
| Composition 4C3 | PCB | NR | NR |
| Composition 5A | PCB | NR | NR |
| Composition 5B | PCB | NR | NR |
| Composition 5C | PCB | NR | NR |
| Composition 5D | PCB | NR | NR |
| HTC1-1 | PCB | NR | NR |
| HTC1-2 | PCB | NR | NR |
| HTC1-3 | PCB | NR | NR |
| HTC1-4 | PCB | NR | NR |
| HTC1-5 | PCB | NR | NR |
| HTC1-6 | PCB | NR | NR |
| HTC1-7 | PCB | NR | NR |
| HTC2-1 | PCB | NR | NR |
| HTC2-2 | PCB | NR | NR |
| HTC2-3 | PCB | NR | NR |
| HTC2-4 | PCB | NR | NR |
| HTC2-5 | PCB | NR | NR |
| HTC2-6 | PCB | NR | NR |
| HTC2-7 | PCB | NR | NR |
| HTC3-1 | PCB | NR | NR |
| HTC3-2 | PCB | NR | NR |
| HTC3-3 | PCB | NR | NR |
| HTC3-4 | PCB | NR | NR |
| HTC3-5 | PCB | NR | NR |
| HTC3-6 | PCB | NR | NR |
| HTC3-7 | PCB | NR | NR |
| HTC4-1 | PCB | NR | NR |
| HTC4-2 | PCB | NR | NR |
| HTC4-3 | PCB | NR | NR |
| HTC4-4 | PCB | NR | NR |
| HTC4-5 | PCB | NR | NR |
| HTC4-6 | PCB | NR | NR |
| HTC4-7 | PCB | NR | NR |
| HTC4-1 | PCB | NR | NR |
| HTC5-B1 | PCB | NR | NR |
| HTC5-B2 | PCB | NR | NR |
| HTC5-B3 | PCB | NR | NR |
| HTC5-B4 | PCB | NR | NR |
| HTC5-B5 | PCB | NR | NR |
| HTC5-B6 | PCB | NR | NR |
| HTC5-B7 | PCB | NR | NR |
| HTC6-1 | PCB | NR | NR |
| HTC6-2 | PCB | NR | NR |
| HTC6-3 | PCB | NR | NR |
| HTC6-4 | PCB | NR | NR |
| HTC6-5 | PCB | NR | NR |
| HTC6-6 | PCB | NR | NR |
| HTC6-7 | PCB | NR | NR |
| Composition 1 | PCB | NR | 50-150 |
| Composition 2A | PCB | NR | 50-150 |
| Composition 3B | PCB | NR | 50-150 |
| Composition 3Bt | PCB | NR | 50-150 |
| Composition 3Bc | PCB | NR | 50-150 |
| Composition 3C | PCB | NR | 50-150 |
| Composition 4A1 | PCB | NR | 50-150 |
| Composition 4A2 | PCB | NR | 50-150 |
| Composition 4A3 | PCB | NR | 50-150 |
| Composition 4B1 | PCB | NR | 50-150 |
| Composition 4B2 | PCB | NR | 50-150 |
| Composition 4B3 | PCB | NR | 50-150 |
| Composition 4C1 | PCB | NR | 50-150 |
| Composition 4C2 | PCB | NR | 50-150 |
| Composition 4C3 | PCB | NR | 50-150 |
| Composition 5A | PCB | NR | 50-150 |
| Composition 5B | PCB | NR | 50-150 |
| Composition 5C | PCB | NR | 50-150 |
| Composition 5D | PCB | NR | 50-150 |
| HTC1-1 | PCB | NR | 50-150 |
| HTC1-2 | PCB | NR | 50-150 |
| HTC1-3 | PCB | NR | 50-150 |
| HTC1-4 | PCB | NR | 50-150 |
| HTC1-5 | PCB | NR | 50-150 |
| HTC1-6 | PCB | NR | 50-150 |
| HTC1-7 | PCB | NR | 50-150 |
| HTC2-1 | PCB | NR | 50-150 |
| HTC2-2 | PCB | NR | 50-150 |
| HTC2-3 | PCB | NR | 50-150 |
| HTC2-4 | PCB | NR | 50-150 |
| HTC2-5 | PCB | NR | 50-150 |
| HTC2-6 | PCB | NR | 50-150 |
| HTC2-7 | PCB | NR | 50-150 |
| HTC3-1 | PCB | NR | 50-150 |
| HTC3-2 | PCB | NR | 50-150 |
| HTC3-3 | PCB | NR | 50-150 |
| HTC3-4 | PCB | NR | 50-150 |
| HTC3-5 | PCB | NR | 50-150 |
| HTC3-6 | PCB | NR | 50-150 |
| HTC3-7 | PCB | NR | 50-150 |
| HTC4-1 | PCB | NR | 50-150 |
| HTC4-2 | PCB | NR | 50-150 |
| HTC4-3 | PCB | NR | 50-150 |
| HTC4-4 | PCB | NR | 50-150 |
| HTC4-5 | PCB | NR | 50-150 |
| HTC4-6 | PCB | NR | 50-150 |
| HTC4-7 | PCB | NR | 50-150 |
| HTC4-1 | PCB | NR | 50-150 |
| HTC5-B1 | PCB | NR | 50-150 |
| HTC5-B2 | PCB | NR | 50-150 |
| HTC5-B3 | PCB | NR | 50-150 |
| HTC5-B4 | PCB | NR | 50-150 |
| HTC5-B5 | PCB | NR | 50-150 |
| HTC5-B6 | PCB | NR | 50-150 |
| HTC5-B7 | PCB | NR | 50-150 |
| HTC6-1 | PCB | NR | 50-150 |
| HTC6-2 | PCB | NR | 50-150 |
| HTC6-3 | PCB | NR | 50-150 |
| HTC6-4 | PCB | NR | 50-150 |
| HTC6-5 | PCB | NR | 50-150 |
| HTC6-6 | PCB | NR | 50-150 |
| HTC6-7 | PCB | NR | 50-150 |
| Composition 1 | PCB | NR | 70-150 |
| Composition 2A | PCB | NR | 70-150 |
| Composition 3B | PCB | NR | 70-150 |
| Composition 3Bt | PCB | NR | 70-150 |
| Composition 3Bc | PCB | NR | 70-150 |
| Composition 3C | PCB | NR | 70-150 |
| Composition 4A1 | PCB | NR | 70-150 |
| Composition 4A2 | PCB | NR | 70-150 |
| Composition 4A3 | PCB | NR | 70-150 |
| Composition 4B1 | PCB | NR | 70-150 |
| Composition 4B2 | PCB | NR | 70-150 |
| Composition 4B3 | PCB | NR | 70-150 |
| Composition 4C1 | PCB | NR | 70-150 |
| Composition 4C2 | PCB | NR | 70-150 |
| Composition 4C3 | PCB | NR | 70-150 |
| Composition 5A | PCB | NR | 70-150 |
| Composition 5B | PCB | NR | 70-150 |
| Composition 5C | PCB | NR | 70-150 |
| Composition 5D | PCB | NR | 70-150 |
| HTC1-1 | PCB | NR | 70-150 |
| HTC1-2 | PCB | NR | 70-150 |
| HTC1-3 | PCB | NR | 70-150 |
| HTC1-4 | PCB | NR | 70-150 |
| HTC1-5 | PCB | NR | 70-150 |
| HTC1-6 | PCB | NR | 70-150 |
| HTC1-7 | PCB | NR | 70-150 |
| HTC2-1 | PCB | NR | 70-150 |
| HTC2-2 | PCB | NR | 70-150 |
| HTC2-3 | PCB | NR | 70-150 |
| HTC2-4 | PCB | NR | 70-150 |
| HTC2-5 | PCB | NR | 70-150 |
| HTC2-6 | PCB | NR | 70-150 |
| HTC2-7 | PCB | NR | 70-150 |
| HTC3-1 | PCB | NR | 70-150 |
| HTC3-2 | PCB | NR | 70-150 |
| HTC3-3 | PCB | NR | 70-150 |
| HTC3-4 | PCB | NR | 70-150 |
| HTC3-5 | PCB | NR | 70-150 |
| HTC3-6 | PCB | NR | 70-150 |
| HTC3-7 | PCB | NR | 70-150 |
| HTC4-1 | PCB | NR | 70-150 |
| HTC4-2 | PCB | NR | 70-150 |
| HTC4-3 | PCB | NR | 70-150 |
| HTC4-4 | PCB | NR | 70-150 |
| HTC4-5 | PCB | NR | 70-150 |
| HTC4-6 | PCB | NR | 70-150 |
| HTC4-7 | PCB | NR | 70-150 |
| HTC4-1 | PCB | NR | 70-150 |
| HTC5-B1 | PCB | NR | 70-150 |
| HTC5-B2 | PCB | NR | 70-150 |
| HTC5-B3 | PCB | NR | 70-150 |
| HTC5-B4 | PCB | NR | 70-150 |
| HTC5-B5 | PCB | NR | 70-150 |
| HTC5-B6 | PCB | NR | 70-150 |
| HTC5-B7 | PCB | NR | 70-150 |
| HTC6-1 | PCB | NR | 70-150 |
| HTC6-2 | PCB | NR | 70-150 |
| HTC6-3 | PCB | NR | 70-150 |
| HTC6-4 | PCB | NR | 70-150 |
| HTC6-5 | PCB | NR | 70-150 |
| HTC6-6 | PCB | NR | 70-150 |
| HTC6-7 | PCB | NR | 70-150 |
| Composition 1 | Server | NR | NR |
| Composition 2A | Server | NR | NR |
| Composition 3B | Server | NR | NR |
| Composition 3Bt | Server | NR | NR |
| Composition 3Bc | Server | NR | NR |
| Composition 3C | Server | NR | NR |
| Composition 4A1 | Server | NR | NR |
| Composition 4A2 | Server | NR | NR |
| Composition 4A3 | Server | NR | NR |
| Composition 4B1 | Server | NR | NR |
| Composition 4B2 | Server | NR | NR |
| Composition 4B3 | Server | NR | NR |
| Composition 4C1 | Server | NR | NR |
| Composition 4C2 | Server | NR | NR |
| Composition 4C3 | Server | NR | NR |
| Composition 5A | Server | NR | NR |
| Composition 5B | Server | NR | NR |
| Composition 5C | Server | NR | NR |
| Composition 5D | Server | NR | NR |
| HTC1-1 | Server | NR | NR |
| HTC1-2 | Server | NR | NR |
| HTC1-3 | Server | NR | NR |
| HTC1-4 | Server | NR | NR |
| HTC1-5 | Server | NR | NR |
| HTC1-6 | Server | NR | NR |
| HTC1-7 | Server | NR | NR |
| HTC2-1 | Server | NR | NR |
| HTC2-2 | Server | NR | NR |
| HTC2-3 | Server | NR | NR |
| HTC2-4 | Server | NR | NR |
| HTC2-5 | Server | NR | NR |
| HTC2-6 | Server | NR | NR |
| HTC2-7 | Server | NR | NR |
| HTC3-1 | Server | NR | NR |
| HTC3-2 | Server | NR | NR |
| HTC3-3 | Server | NR | NR |
| HTC3-4 | Server | NR | NR |
| HTC3-5 | Server | NR | NR |
| HTC3-6 | Server | NR | NR |
| HTC3-7 | Server | NR | NR |
| HTC4-1 | Server | NR | NR |
| HTC4-2 | Server | NR | NR |
| HTC4-3 | Server | NR | NR |
| HTC4-4 | Server | NR | NR |
| HTC4-5 | Server | NR | NR |
| HTC4-6 | Server | NR | NR |
| HTC4-7 | Server | NR | NR |
| HTC4-1 | Server | NR | NR |
| HTC5-B1 | Server | NR | NR |
| HTC5-B2 | Server | NR | NR |
| HTC5-B3 | Server | NR | NR |
| HTC5-B4 | Server | NR | NR |
| HTC5-B5 | Server | NR | NR |
| HTC5-B6 | Server | NR | NR |
| HTC5-B7 | Server | NR | NR |
| HTC6-1 | Server | NR | NR |
| HTC6-2 | Server | NR | NR |
| HTC6-3 | Server | NR | NR |
| HTC6-4 | Server | NR | NR |
| HTC6-5 | Server | NR | NR |
| HTC6-6 | Server | NR | NR |
| HTC6-7 | Server | NR | NR |
| Composition 1 | Server | NR | 50-150 |
| Composition 2A | Server | NR | 50-150 |
| Composition 3B | Server | NR | 50-150 |
| Composition 3Bt | Server | NR | 50-150 |
| Composition 3Bc | Server | NR | 50-150 |
| Composition 3C | Server | NR | 50-150 |
| Composition 4A1 | Server | NR | 50-150 |
| Composition 4A2 | Server | NR | 50-150 |
| Composition 4A3 | Server | NR | 50-150 |
| Composition 4B1 | Server | NR | 50-150 |
| Composition 4B2 | Server | NR | 50-150 |
| Composition 4B3 | Server | NR | 50-150 |
| Composition 4C1 | Server | NR | 50-150 |
| Composition 4C2 | Server | NR | 50-150 |
| Composition 4C3 | Server | NR | 50-150 |
| Composition 5A | Server | NR | 50-150 |
| Composition 5B | Server | NR | 50-150 |
| Composition 5C | Server | NR | 50-150 |
| Composition 5D | Server | NR | 50-150 |
| HTC1-1 | Server | NR | 50-150 |
| HTC1-2 | Server | NR | 50-150 |
| HTC1-3 | Server | NR | 50-150 |
| HTC1-4 | Server | NR | 50-150 |
| HTC1-5 | Server | NR | 50-150 |
| HTC1-6 | Server | NR | 50-150 |
| HTC1-7 | Server | NR | 50-150 |
| HTC2-1 | Server | NR | 50-150 |
| HTC2-2 | Server | NR | 50-150 |
| HTC2-3 | Server | NR | 50-150 |
| HTC2-4 | Server | NR | 50-150 |
| HTC2-5 | Server | NR | 50-150 |
| HTC2-6 | Server | NR | 50-150 |
| HTC2-7 | Server | NR | 50-150 |
| HTC3-1 | Server | NR | 50-150 |
| HTC3-2 | Server | NR | 50-150 |
| HTC3-3 | Server | NR | 50-150 |
| HTC3-4 | Server | NR | 50-150 |
| HTC3-5 | Server | NR | 50-150 |
| HTC3-6 | Server | NR | 50-150 |
| HTC3-7 | Server | NR | 50-150 |
| HTC4-1 | Battery | NR | 50-150 |
| HTC4-2 | Battery | NR | 50-150 |
| HTC4-3 | Battery | NR | 50-150 |
| HTC4-4 | Battery | NR | 50-150 |
| HTC4-5 | Battery | NR | 50-150 |
| HTC4-6 | Battery | NR | 50-150 |
| HTC4-7 | Battery | NR | 50-150 |
| HTC4-1 | Battery | NR | 50-150 |
| HTC5-B1 | Battery | NR | 50-150 |
| HTC5-B2 | Battery | NR | 50-150 |
| HTC5-B3 | Battery | NR | 50-150 |
| HTC5-B4 | Battery | NR | 50-150 |
| HTC5-B5 | Battery | NR | 50-150 |
| HTC5-B6 | Battery | NR | 50-150 |
| HTC5-B7 | Battery | NR | 50-150 |
| HTC6-1 | Battery | NR | 50-150 |
| HTC6-2 | Battery | NR | 50-150 |
| HTC6-3 | Battery | NR | 50-150 |
| HTC6-4 | Battery | NR | 50-150 |
| HTC6-5 | Battery | NR | 50-150 |
| HTC6-6 | Battery | NR | 50-150 |
| HTC6-7 | Battery | NR | 50-150 |
| Composition 1 | Server | NR | 70-120 |
| Composition 2A | Server | NR | 70-120 |
| Composition 3B | Server | NR | 70-120 |
| Composition 3Bt | Server | NR | 70-120 |
| Composition 3Bc | Server | NR | 70-120 |
| Composition 3C | Server | NR | 70-120 |
| Composition 4A1 | Server | NR | 50-150 |
| Composition 4A2 | Server | NR | 70-120 |
| Composition 4A3 | Server | NR | 70-120 |
| Composition 4B1 | Server | NR | 70-120 |
| Composition 4B2 | Server | NR | 70-120 |
| Composition 4B3 | Server | NR | 70-120 |
| Composition 4C1 | Server | NR | 70-120 |
| Composition 4C2 | Server | NR | 70-120 |
| Composition 4C3 | Server | NR | 70-120 |
| Composition 5A | Server | NR | 70-120 |
| Composition 5B | Server | NR | 70-120 |
| Composition 5C | Server | NR | 70-120 |
| Composition 5D | Server | NR | 70-120 |
| HTC1-1 | Server | NR | 70-120 |
| HTC1-2 | Server | NR | 70-120 |
| HTC1-3 | Server | NR | 70-120 |
| HTC1-4 | Server | NR | 70-120 |
| HTC1-5 | Server | NR | 70-120 |
| HTC1-6 | Server | NR | 70-120 |
| HTC1-7 | Server | NR | 70-120 |
| HTC2-1 | Server | NR | 70-120 |
| HTC2-2 | Server | NR | 70-120 |
| HTC2-3 | Server | NR | 70-120 |
| HTC2-4 | Server | NR | 70-120 |
| HTC2-5 | Server | NR | 70-120 |
| HTC2-6 | Server | NR | 70-120 |
| HTC2-7 | Server | NR | 70-120 |
| HTC3-1 | Server | NR | 70-120 |
| HTC3-2 | Server | NR | 70-120 |
| HTC3-3 | Server | NR | 70-120 |
| HTC3-4 | Server | NR | 70-120 |
| HTC3-5 | Server | NR | 70-120 |
| HTC3-6 | Server | NR | 70-120 |
| HTC3-7 | Server | NR | 70-120 |
| HTC4-1 | Server | NR | 70-120 |
| HTC4-2 | Server | NR | 70-120 |
| HTC4-3 | Server | NR | 70-120 |
| HTC4-4 | Server | NR | 70-120 |
| HTC4-5 | Server | NR | 70-120 |
| HTC4-6 | Server | NR | 70-120 |
| HTC4-7 | Server | NR | 70-120 |
| HTC4-1 | Server | NR | 70-120 |
| HTC5-B1 | Server | NR | 70-120 |
| HTC5-B2 | Server | NR | 70-120 |
| HTC5-B3 | Server | NR | 70-120 |
| HTC5-B4 | Server | NR | 70-120 |
| HTC5-B5 | Server | NR | 70-120 |
| HTC5-B6 | Server | NR | 70-120 |
| HTC5-B7 | Server | NR | 70-120 |
| HTC6-1 | Server | NR | 70-120 |
| HTC6-2 | Server | NR | 70-120 |
| HTC6-3 | Server | NR | 70-120 |
| HTC6-4 | Server | NR | 70-120 |
| HTC6-5 | Server | NR | 70-120 |
| HTC6-6 | Server | NR | 70-120 |
| HTC6-7 | Server | NR | 70-120 |
| Composition 1 | Server | data center | NR |
| Composition 2A | Server | data center | NR |
| Composition 3B | Server | data center | NR |
| Composition 3Bt | Server | data center | NR |
| Composition 3Bc | Server | data center | NR |
| Composition 3C | Server | data center | NR |
| Composition 4A1 | Server | data center | NR |
| Composition 4A2 | Server | data center | NR |
| Composition 4A3 | Server | data center | NR |
| Composition 4B1 | Server | data center | NR |
| Composition 4B2 | Server | data center | NR |
| Composition 4B3 | Server | data center | NR |
| Composition 4C1 | Server | data center | NR |
| Composition 4C2 | Server | data center | NR |
| Composition 4C3 | Server | data center | NR |
| Composition 5A | Server | data center | NR |
| Composition 5B | Server | data center | NR |
| Composition 5C | Server | data center | NR |
| Composition 5D | Server | data center | NR |
| HTC1-1 | Server | data center | NR |
| HTC1-2 | Server | data center | NR |
| HTC1-3 | Server | data center | NR |
| HTC1-4 | Server | data center | NR |
| HTC1-5 | Server | data center | NR |
| HTC1-6 | Server | data center | NR |
| HTC1-7 | Server | data center | NR |
| HTC2-1 | Server | data center | NR |
| HTC2-2 | Server | data center | NR |
| HTC2-3 | Server | data center | NR |
| HTC2-4 | Server | data center | NR |
| HTC2-5 | Server | data center | NR |
| HTC2-6 | Server | data center | NR |
| HTC2-7 | Server | data center | NR |
| HTC3-1 | Server | data center | NR |
| HTC3-2 | Server | data center | NR |
| HTC3-3 | Server | data center | NR |
| HTC3-4 | Server | data center | NR |
| HTC3-5 | Server | data center | NR |
| HTC3-6 | Server | data center | NR |
| HTC3-7 | Server | data center | NR |
| HTC4-1 | Server | data center | NR |
| HTC4-2 | Server | data center | NR |
| HTC4-3 | Server | data center | NR |
| HTC4-4 | Server | data center | NR |
| HTC4-5 | Server | data center | NR |
| HTC4-6 | Server | data center | NR |
| HTC4-7 | Server | data center | NR |
| HTC4-1 | Server | data center | NR |
| HTC5-B1 | Server | data center | NR |
| HTC5-B2 | Server | data center | NR |
| HTC5-B3 | Server | data center | NR |
| HTC5-B4 | Server | data center | NR |
| HTC5-B5 | Server | data center | NR |
| HTC5-B6 | Server | data center | NR |
| HTC5-B7 | Server | data center | NR |
| HTC6-1 | Server | data center | NR |
| HTC6-2 | Server | data center | NR |
| HTC6-3 | Server | data center | NR |
| HTC6-4 | Server | data center | NR |
| HTC6-5 | Server | data center | NR |
| HTC6-6 | Server | data center | NR |
| HTC6-7 | Server | data center | NR |
| Composition 1 | Server | data center | 50-150 |
| Composition 2A | Server | data center | 50-150 |
| Composition 3B | Server | data center | 50-150 |
| Composition 3Bt | Server | data center | 50-150 |
| Composition 3Bc | Server | data center | 50-150 |
| Composition 3C | Server | data center | 50-150 |
| Composition 4A1 | Server | data center | 50-150 |
| Composition 4A2 | Server | data center | 50-150 |
| Composition 4A3 | Server | data center | 50-150 |
| Composition 4B1 | Server | data center | 50-150 |
| Composition 4B2 | Server | data center | 50-150 |
| Composition 4B3 | Server | data center | 50-150 |
| Composition 4C1 | Server | data center | 50-150 |
| Composition 4C2 | Server | data center | 50-150 |
| Composition 4C3 | Server | data center | 50-150 |
| Composition 5A | Server | data center | 50-150 |
| Composition 5B | Server | data center | 50-150 |
| Composition 5C | Server | data center | 50-150 |
| Composition 5D | Server | data center | 50-150 |
| HTC1-1 | Server | data center | 50-150 |
| HTC1-2 | Server | data center | 50-150 |
| HTC1-3 | Server | data center | 50-150 |
| HTC1-4 | Server | data center | 50-150 |
| HTC1-5 | Server | data center | 50-150 |
| HTC1-6 | Server | data center | 50-150 |
| HTC1-7 | Server | data center | 50-150 |
| HTC2-1 | Server | data center | 50-150 |
| HTC2-2 | Server | data center | 50-150 |
| HTC2-3 | Server | data center | 50-150 |
| HTC2-4 | Server | data center | 50-150 |
| HTC2-5 | Server | data center | 50-150 |
| HTC2-6 | Server | data center | 50-150 |
| HTC2-7 | Server | data center | 50-150 |
| HTC3-1 | Server | data center | 50-150 |
| HTC3-2 | Server | data center | 50-150 |
| HTC3-3 | Server | data center | 50-150 |
| HTC3-4 | Server | data center | 50-150 |
| HTC3-5 | Server | data center | 50-150 |
| HTC3-6 | Server | data center | 50-150 |
| HTC3-7 | Server | data center | 50-150 |
| HTC4-1 | Server | data center | 50-150 |
| HTC4-2 | Server | data center | 50-150 |
| HTC4-3 | Server | data center | 50-150 |
| HTC4-4 | Server | data center | 50-150 |
| HTC4-5 | Server | data center | 50-150 |
| HTC4-6 | Server | data center | 50-150 |
| HTC4-7 | Server | data center | 50-150 |
| HTC4-1 | Server | data center | 50-150 |
| HTC5-B1 | Server | data center | 50-150 |
| HTC5-B2 | Server | data center | 50-150 |
| HTC5-B3 | Server | data center | 50-150 |
| HTC5-B4 | Server | data center | 50-150 |
| HTC5-B5 | Server | data center | 50-150 |
| HTC5-B6 | Server | data center | 50-150 |
| HTC5-B7 | Server | data center | 50-150 |
| HTC6-1 | Server | data center | 50-150 |
| HTC6-2 | Server | data center | 50-150 |
| HTC6-3 | Server | data center | 50-150 |
| HTC6-4 | Server | data center | 50-150 |
| HTC6-5 | Server | data center | 50-150 |
| HTC6-6 | Server | data center | 50-150 |
| HTC6-7 | Server | data center | 50-150 |
| Composition 1 | Server | data center | 70-120 |
| Composition 2A | Server | data center | 70-120 |
| Composition 3B | Server | data center | 70-120 |
| Composition 3Bt | Server | data center | 70-120 |
| Composition 3Bc | Server | data center | 70-120 |
| Composition 3C | Server | data center | 70-120 |
| Composition 4A1 | Server | data center | 70-120 |
| Composition 4A2 | Server | data center | 70-120 |
| Composition 4A3 | Server | data center | 70-120 |
| Composition 4B1 | Server | data center | 70-120 |
| Composition 4B2 | Server | data center | 70-120 |
| Composition 4B3 | Server | data center | 70-120 |
| Composition 4C1 | Server | data center | 70-120 |
| Composition 4C2 | Server | data center | 70-120 |
| Composition 4C3 | Server | data center | 70-120 |
| Composition 5A | Server | data center | 70-120 |
| Composition 5B | Server | data center | 70-120 |
| Composition 5C | Server | data center | 70-120 |
| Composition 5D | Server | data center | 70-120 |
| HTC1-1 | Server | data center | 70-120 |
| HTC1-2 | Server | data center | 70-120 |
| HTC1-3 | Server | data center | 70-120 |
| HTC1-4 | Server | data center | 70-120 |
| HTC1-5 | Server | data center | 70-120 |
| HTC1-6 | Server | data center | 70-120 |
| HTC1-7 | Server | data center | 70-120 |
| HTC2-1 | Server | data center | 70-120 |
| HTC2-2 | Server | data center | 70-120 |
| HTC2-3 | Server | data center | 70-120 |
| HTC2-4 | Server | data center | 70-120 |
| HTC2-5 | Server | data center | 70-120 |
| HTC2-6 | Server | data center | 70-120 |
| HTC2-7 | Server | data center | 70-120 |
| HTC3-1 | Server | data center | 70-120 |
| HTC3-2 | Server | data center | 70-120 |
| HTC3-3 | Server | data center | 70-120 |
| HTC3-4 | Server | data center | 70-120 |
| HTC3-5 | Server | data center | 70-120 |
| HTC3-6 | Server | data center | 70-120 |
| HTC3-7 | Server | data center | 70-120 |
| HTC4-1 | Server | data center | 70-120 |
| HTC4-2 | Server | data center | 70-120 |
| HTC4-3 | Server | data center | 70-120 |
| HTC4-4 | Server | data center | 70-120 |
| HTC4-5 | Server | data center | 70-120 |
| HTC4-6 | Server | data center | 70-120 |
| HTC4-7 | Server | data center | 70-120 |
| HTC4-1 | Server | data center | 70-120 |
| HTC5-B1 | Server | data center | 70-120 |
| HTC5-B2 | Server | data center | 70-120 |
| HTC5-B3 | Server | data center | 70-120 |
| HTC5-B4 | Server | data center | 70-120 |
| HTC5-B5 | Server | data center | 70-120 |
| HTC5-B6 | Server | data center | 70-120 |
| HTC5-B7 | Server | data center | 70-120 |
| HTC6-1 | Server | data center | 70-120 |
| HTC6-C2 | Server | data center | 70-120 |
| HTC6-3 | Server | data center | 70-120 |
| HTC6-4 | Server | data center | 70-120 |
| HTC6-5 | Server | data center | 70-120 |
| HTC6-6 | Server | data center | 70-120 |
| HTC6-7 | Server | data center | 70-120 |

### Heat Pipe Cooling and Heating

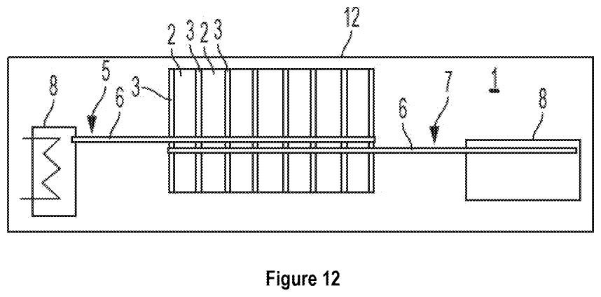

An example of the present heat transfer methods, including Heat Transfer Methods 1, using the presents compositions, including Compositions 1-5 and HTCs 1-6, is now described with respect to FIG. 12, which is a specific example of a heat pipe in an energy storage assembly 1 according to one exemplary embodiment of the invention. The energy storage assembly 1 may be part of a motor vehicle 12, in particular of a hybrid or electric vehicle, and is provided to supply electric power to electric consumers, like e.g. an electrical drive unit (not shown), on the motor vehicle side. The energy storage assembly 1 includes a plurality of electrical energy stores. 2. The electrical energy stores 2 are electrically connected via an electric connection element (not shown), in particular in the form of a conductive rail or conductor rail (“bus bar”), i.e., connected in series or in parallel. The electric connection element contacts hereby corresponding electrical connectors (not shown) arranged on respective exposed outer wall sections of corresponding energy storage housings (not shown) of the energy stores 2 in parallel alignment arranged adjacent to one another to thereby form an energy store stack (“stack”). Plate-shaped spacer elements 3 are respectively arranged between the energy stores 2 to separate them and at the same time thermally conductive properties. The spacer elements 3 thus provide, on one hand, spacing between immediately adjacent energy stores 2 so that immediately adjacent energy stores 2 do not contact each other electrically or mechanically. On the other hand, the spacer elements 3 act as a result of their thermally conductive properties as heat conductors for the purpose of cooling the energy stores 2 or the energy storage assembly 1 by dissipating heat, particularly from the contacting energy stores 2, or, for the purpose of heating the energy stores 2 or energy storage assembly 1 by supplying heat, particularly to the contacting energy stores 2. A heat pipe 4 of a first heat pipe assembly 5 and of a heat pipe 6 of a second heat pipe assembly 7 are provide. The heat pipes 4, 6 thus extend along this side surface of the energy store stack and are thermally coupled to the spacer elements 3, respectively. The spacer elements 3 thus form a thermal bridge between the heat pipes 4 of the first heat pipe assembly 5 and the heat pipes 6 of the second heat pipe assembly 7, on one hand, and the energy stores 2, on the other hand. The respective heat pipes 4 of the first heat pipe assembly 5 are arranged and aligned such as to be thermally coupled with respective evaporation zones, in which a contained thermal management fluid of the present invention, including particularly each of Compositions 1-5 and HTCs 1-6, can be evaporated, to the spacer elements 3. Heat (evaporation heat) required for the evaporation of the present TMF is thus removed from the spacer elements 3 or via the spacer elements 3 from the energy stores 2. The energy stores 2, including the energy storage assembly 1, can thus be cooled via the heat pipes 4 of the first heat pipe assembly 5. Also, the respective condensation zones of the heat pipes 4 of the first heat pipe assembly 5, in which condensation zones a contained gaseous thermal management fluid of the present invention, including particularly each of Compositions 1-5 and HTCs 1-6, can be condensed, are thermally coupled with a heat sink 8 in the form of a motor-vehicle-side heat exchanger. Heat (condensation heat) generated during condensation of the present compositions and working fluids can thus be transferred to the heat sink 8. The heat exchanger can be part of the energy storage assembly 1, i.e., belonging to or associated with the energy storage assembly 1. The respective heat pipes 6 of the second heat pipe assembly 7 are arranged and aligned such as to be thermally coupled with their respective condensation zones, in which the contained gaseous compositions and working fluids of the present invention can be condensed, to the spacer elements 3. Heat (condensation heat) can thus be transferred during condensation of the present compositions and working fluids to the spacer elements 3 or via the spacer elements 3 to the energy stores 2. Thus, the energy stores 2 and the energy storage assembly 1, can be heated via the heat pipes 6 of the second heat pipe assembly 7. Also, the respective evaporation zones of the heat pipes 6 of the second heat pipe assembly 7, in which evaporation zones a contained compositions and working fluids of the present invention can be evaporated, are thermally coupled with a heat source 9 in the form of a functional component, i.e., e.g., a charger or a control device or a control electronics, associated to the energy storage assembly 1. Heat (evaporation heat) required for the evaporation can thus be removed from the heat source 9. Thus, the functional component can be cooled via the heat pipes 6 of the second heat pipe assembly 7. The two heat pipe assemblies 5, 7, and their associated heat pipes 4, 6 enables implementation of a temperature control device for controlling the temperature, i.e., for heating or cooling, of the energy stores 2 of the energy storage assembly 1. The heat pipes useful according to the present invention include both gravity return heat pipe, capillary return heat pipe and gravity/capillary return heat pipes.

### Organic Rankine Cycle

As discussed above, a heat transfer fluid of the present invention, including each of Compositions 1-5 and HTCs 1-6, can be used in an Organic Rankine cycle. Organic Rankine cycle systems are known to be a simple and reliable means to convert heat energy into mechanical energy in the form of shaft power. In industrial settings, it may be possible to use flammable working fluids such as toluene and pentane, particularly when the industrial setting has large quantities of flammables already on site in processes or storage. However, for instances where the risk associated with use of a flammable and/or toxic working fluid is not acceptable, such as power generation in populous areas or near buildings, it is necessary or at least highly desirable to use non-flammable and/or non-toxic refrigerants as the working fluid. There is also a drive in the industry for these materials to be environmentally acceptable in terms of GWP.

The process for recovering waste heat in an Organic Rankine cycle according to the present invention preferably involves pumping liquid-phase working fluid of the present invention, including each of Compositions 1-5 and HTCs 1-6, through a boiler where an external (waste) heat source, such as a process stream, heats the working fluid causing it to evaporate into a saturated or superheated vapor. This vapor is expanded through a turbine wherein the waste heat energy is converted into mechanical energy. Subsequently, the vapor phase working fluid is condensed to a liquid and pumped back to the boiler in order to repeat the heat extraction cycle.

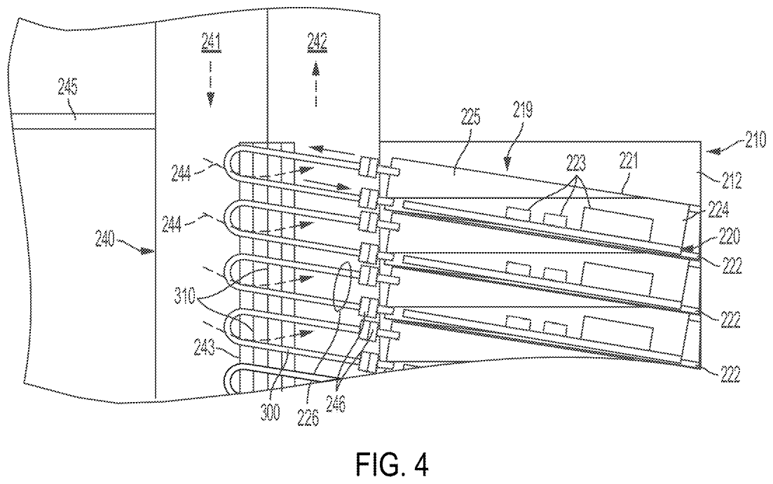

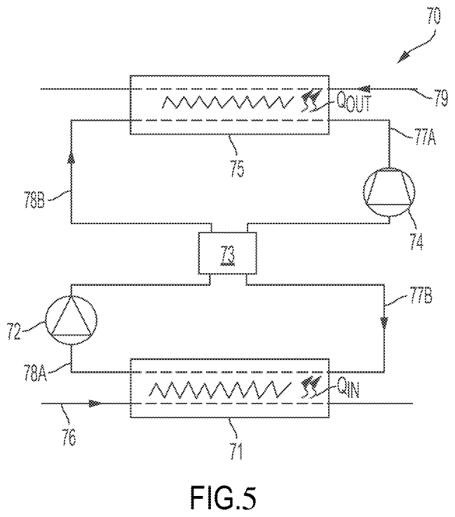

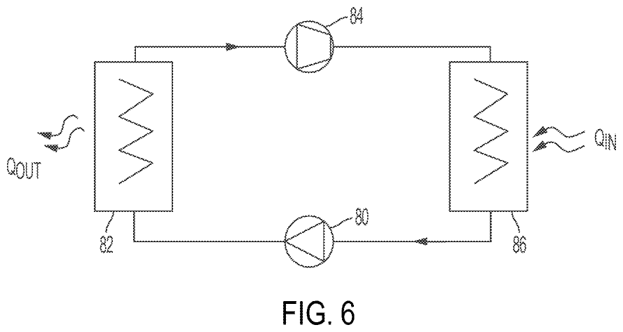

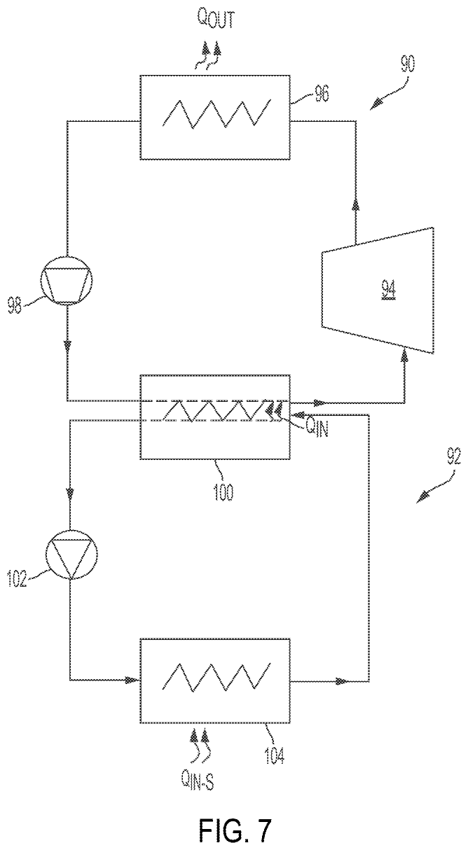

Referring to FIG. 4, in an exemplary organic Rankine cycle system 70, working fluid of the present invention, including each of Compositions 1-5 and HTCs 1-6, is circulated between an evaporator 71 and a condenser 75, with a pump 72 and an expansion device 74 functionally disposed therebetween. In the illustrated embodiment, an external flow of fluid is directed to evaporator 71 via external warm conduit 76. External warm conduit 76 may carry fluid from a warm heat source, such as a waste heat source from industrial processes (e.g., power generation), flue gases, exhaust gases, geothermal sources, etc.

Evaporator 71 is preferably configured as a heat exchanger which may include, e.g., a series of thermally connected, but fluidly isolated, tubes carrying fluid from warm conduit 76 and fluid from working fluid conduit 77B respectively. Thus, evaporator 71 facilitates the transfer of heat QIN from the warm fluid arriving from external warm conduit 76 to the relatively cooler (e.g., “cold”) working fluid arriving from expansion device 74 via working fluid conduit 77B.

The working fluid of the present invention, including each of Compositions 1-5 and each of the working fluids in Table 2, thus exits from evaporator 71, having been warmed by the absorption of heat QIN, and then travels through working fluid conduit 78A to pump 72. Pump 72 pressurizes the working fluid, thereby further warming the fluid through external energy inputs (e.g., electricity). The resulting “hot” fluid passes to an input of condenser 75 via conduit 78B, optionally via a regenerator 73 as described below.

Condenser 75 is configured as a heat exchanger similar to evaporator 71, and may include, e.g., a series of thermally connected, but fluidly isolated, tubes carrying fluid from cool conduit 79 and fluid from working fluid conduit 78B respectively. Condenser 75 facilitates the transfer of heat QOUT to the cool fluid arriving from external cool conduit 79 to the relatively warmer (e.g., “hot”) working fluid of the present invention, including each of Compositions 1-5 and HTCs 1-6, arriving from pump 72 via working fluid conduit 78B.

The working fluid of the present invention, including each of Compositions 1-5 and HTCs 1-6, exiting from condenser 75, having thus been cooled by the loss of heat QOUT, then travels through working fluid conduit 77A to expansion device 74. Expansion device 74 allows the working fluid to expand, thereby further cooling the fluid. At this stage, the fluid of the present invention, including each of Compositions 1-5 and HTCs 1-6, may perform work, e.g., by driving a turbine. The resulting “cold” fluid passes to an input of evaporator 71 via conduit 77B, optionally via a regenerator 73 as described below, and the cycle begins anew.

Thus, working fluid conduits 77A, 77B, 78A and 78B define a closed loop such that the working fluid contained therein may be reused indefinitely, or until routing maintenance is required.

In the illustrated embodiment, regenerator 73 may be functionally disposed between evaporator 71 and condenser 75. Regenerator 73 allows the “hot” working fluid of the present invention, including each of Compositions 1-5 and HTCs 1-6, exiting from pump 72 and the “cold” working fluid issued from expansion device 74 to exchange some heat, potentially with a time lag between deposit of heat from the hot working fluid and release of that heat to the cold working fluid. In some applications, this can increase the overall thermal efficiency of Rankine cycle system 70.

Therefore, the invention relates to an organic Rankine cycle comprising a working fluid of the present invention, including each of Compositions 1-5 and HTCs 1-6.

The invention further relates to the use of a working fluid of the invention, including each of Compositions 1-5 and HTCs 1-6, in an Organic Rankine Cycle.

The invention also provides a process for converting thermal energy to mechanical energy in a Rankine cycle, the method comprising the steps of i) vaporizing a working fluid of the invention, including each of Compositions 1-5 and HTCs 1-6, with a heat source and expanding the resulting vapor, then ii) cooling the working fluid with a heat sink to condense the vapor, wherein the working fluid is a refrigerant or heat transfer composition of the invention, including each of Compositions 1-5 and HTCs 1-6.

The mechanical work may be transmitted to an electrical device such as a generator to produce electrical power.

The heat source maybe provided by, for example, a thermal energy source selected from industrial waste heat, solar energy, geothermal hot water, low pressure steam, distributed power generation equipment utilizing fuel cells, prime movers, or an internal combustion engine.

The low pressure steam is preferably a low pressure geothermal steam or is provided by a fossil fuel powered electrical generating power plant.

The heat source is preferably provided by a thermal energy source selected from industrial waste heat, or an internal combustion engine.

It will be appreciated that the heat source temperatures can vary widely, for example from about 90 °C to >800 °C, and can be dependent upon a myriad of factors including geography, time of year, etc. for certain combustion gases and some fuel cells.

Systems based on sources such as waste water or low pressure steam from, e.g., a plastics manufacturing plants and/or from chemical or other industrial plant, petroleum refinery, and related word forms, as well as geothermal sources, may have source temperatures that are at or below about 175 °C or at or below about 100 °C, and in some cases as low as about 90 °C or even as low as about 80 °C. Gaseous sources of heat such as exhaust gas from combustion process or from any heat source where subsequent treatments to remove particulates and/or corrosive species result in low temperatures may also have source temperatures that are at or below 200 °C, at or below about 175 °C, at or below about 130 °C, at or below about 120 °C, at or below about 100 °C, at or below about 100 °C, and in some cases as low as about 90 °C or even as low as about 80 °C.

However, it is preferred in some applications that the heat source has a temperature of at least about 200 °C, for example of from about 200 °C to about 400 °C.

In an alternative preferred embodiment, the heat source has a temperature of from 400 to 800 °C, more preferably 400 to 600 °C.

The following table identifies preferred high temperature heat pump methods of the present invention (identified by the HTHP Method numbers in column 1) using: (1) the refrigerants of the present invention (identified in column 2 by numbers corresponding to the Compositions defined above or by the HTC Number as defined above); (2) a direct expansion evaporator; (3) a vapor injector economizer; and (4) having the operating parameters as specified in the Table 4 below.

TABLE 4
ORGANIC RANKINE CYCLE METHOD SOURCES AND CONDITIONS

| ORC | | | Heat Source |
|---|---|---|---|
| Method | Composition. | | Temperature, |
| No. | No./HTC No. | Waste Heat Source | °C |
| 1A | 1 | Industrial waste heat | NR |
| 1B | 1 | Industrial waste heat | 90-800 |
| 1C | 1 | Industrial waste heat | 90-400 |
| 1D | 1 | Industrial waste heat | 90-200 |
| 2A | 1 | Solar energy | NR |
| 2B | 1 | Solar energy | 100-400 |
| 2C | 1 | Solar energy | 150-350 |
| 2D | 1 | Solar energy | 200-300 |
| 3A | 1 | Geothermal water | NR |
| 3B | 1 | Geothermal water | 50-350 |
| 3C | 1 | Geothermal water | 100-300 |
| 3D | 1 | Geothermal water | 125-275 |
| 4A | 1 | Low pressure steam | NR |
| 4B | 1 | Low pressure steam | =<121 |
| 4C | 1 | Low pressure steam | =<115 |
| 4D | 1 | Low pressure steam | =<110 |
| 5A | 1 | distributed power generation equipment using fuel cells | NR |
| 5B | 1 | distributed power generation equipment using fuel cells | 60-200 |
| 5C | 1 | distributed power generation equipment using fuel cells | 80-200 |
| 5D | 1 | distributed power generation equipment using fuel cells | 100-200 |
| 6A | 1 | Exhaust gas from internaI combustion engine | NR |
| 6B | 1 | Exhaust gas from internaI combustion engine | =<500 |
| 6C | 1 | Exhaust gas from internaI combustion engine | =<450 |
| 6D | 1 | Exhaust gas from internaI combustion engine | =<400 |
| 7A | 2A | Industrial waste heat | NR |
| 7B | 2A | Industrial waste heat | 90-800 |
| 7C | 2A | Industrial waste heat | 90-400 |
| 7D | 2A | Industrial waste heat | 90-200 |
| 8A | 2A | Solar energy | NR |
| 8B | 2A | Solar energy | 100-400 |
| 8C | 2A | Solar energy | 150-350 |
| 8D | 2A | Solar energy | 200-300 |
| 9A | 2A | Geothermal water | NR |
| 9B | 2A | Geothermal water | 50-350 |
| 9C | 2A | Geothermal water | 100-300 |
| 9D | 2A | Geothermal water | 125-275 |
| 10A | 2A | Low pressure steam | NR |
| 10B | 2A | Low pressure steam | =<121 |
| 10C | 2A | Low pressure steam | =<115 |
| 10D | 2A | Low pressure steam | =<110 |
| 11A | 2A | distributed power generation equipment using fuel cells | NR |
| 11B | 2A | distributed power generation equipment using fuel cells | 60-200 |
| 11C | 2A | distributed power generation equipment using fuel cells | 80-200 |
| 11D | 2A | distributed power generation equipment using fuel cells | 100-200 |
| 12A | 3B | Industrial waste heat | NR |
| 12B | 3B | Industrial waste heat | 90-800 |
| 12C | 3B | Industrial waste heat | 90-400 |
| 12D | 3B | Industrial waste heat | 90-200 |
| 13A | 3B | Solar energy | NR |
| 13B | 3B | Solar energy | 100-400 |
| 13C | 3B | Solar energy | 150-350 |
| 13D | 3B | Solar energy | 200-300 |
| 14A | 3B | Geothermal water | NR |
| 14B | 3B | Geothermal water | 50-350 |
| 14C | 3B | Geothermal water | 100-300 |
| 14D | 3B | Geothermal water | 125-275 |
| 15A | 3B | Low pressure steam | NR |
| 15B | 3B | Low pressure steam | =<121 |
| 15C | 3B | Low pressure steam | =<115 |
| 15D | 3B | Low pressure steam | =<110 |
| 16A | 3B | distributed power generation equipment using fuel cells | NR |
| 16B | 3B | distributed power generation equipment using fuel cells | 60-200 |
| 16C | 3B | distributed power generation equipment using fuel cells | 80-200 |
| 16D | 3B | distributed power generation equipment using fuel cells | 100-200 |
| 17A | 3B | Exhaust gas from internaI combustion engine | NR |
| 17B | 3B | Exhaust gas from internaI combustion engine | =<500 |
| 17C | 3B | Exhaust gas from internaI combustion engine | =<450 |
| 17D | 3B | Exhaust gas from internaI combustion engine | =<400 |
| 18A | 3B | Industrial waste heat | NR |
| 18B | 3B | Industrial waste heat | 90-800 |
| 18C | 3B | Industrial waste heat | 90-400 |
| 18D | 3B | Industrial waste heat | 90-200 |
| 19A | 3Bt | Solar energy | NR |
| 19B | 3Bt | Solar energy | 100-400 |
| 19C | 3Bt | Solar energy | 150-350 |
| 19D | 3Bt | Solar energy | 200-300 |
| 20A | 3Bt | Geothermal water | NR |
| 20B | 3Bt | Geothermal water | 50-350 |
| 20C | 3Bt | Geothermal water | 100-300 |
| 20D | 3Bt | Geothermal water | 125-275 |
| 21A | 3Bt | Low pressure steam | NR |
| 21B | 3Bt | Low pressure steam | =<121 |
| 21C | 3Bt | Low pressure steam | =<115 |
| 21D | 3Bt | Low pressure steam | =<110 |
| 22A | 3Bt | distributed power generation equipment using fuel cells | NR |
| 22B | 3Bt | distributed power generation equipment using fuel cells | 60-200 |
| 22C | 3Bt | distributed power generation equipment using fuel cells | 80-200 |
| 22D | 3Bt | distributed power generation equipment using fuel cells | 100-200 |
| 23A | 3Bt | Exhaust gas from internaI combustion engine | NR |
| 23B | 3Bt | Exhaust gas from internaI combustion engine | =<500 |
| 23C | 3Bt | Exhaust gas from internaI combustion engine | =<450 |
| 23D | 3Bt | Exhaust gas from internaI combustion engine | =<400 |
| 24A | 3C | Industrial waste heat | NR |
| 24B | 3C | Industrial waste heat | 90-800 |
| 24C | 3C | Industrial waste heat | 90-400 |
| 24D | 3C | Industrial waste heat | 90-200 |
| 25A | 3C | Solar energy | NR |
| 25B | 3C | Solar energy | 100-400 |
| 25C | 3C | Solar energy | 150-350 |
| 25D | 3C | Solar energy | 200-300 |
| 26A | 3C | Geothermal water | NR |
| 26B | 3C | Geothermal water | 50-350 |
| 26C | 3C | Geothermal water | 100-300 |
| 26D | 3C | Geothermal water | 125-275 |
| 27A | 3C | Low pressure steam | NR |
| 27B | 3C | Low pressure steam | =<121 |
| 27C | 3C | Low pressure steam | =<115 |
| 27D | 3C | Low pressure steam | =<110 |
| 28A | 3C | distributed power generation equipment using fuel cells | NR |
| 28B | 3C | distributed power generation equipment using fuel cells | 60-200 |
| 28C | 3C | distributed power generation equipment using fuel cells | 80-200 |
| 28D | 3C | distributed power generation equipment using fuel cells | 100-200 |
| 29A | 3C | Exhaust gas from internaI combustion engine | NR |
| 29B | 3C | Exhaust gas from internaI combustion engine | =<500 |
| 29C | 3C | Exhaust gas from internaI combustion engine | =<450 |
| 29D | 3C | Exhaust gas from internaI combustion engine | =<400 |
| 38A | 3Bc | Industrial waste heat | NR |
| 38B | 3Bc | Industrial waste heat | 90-800 |
| 38C | 3Bc | Industrial waste heat | 90-400 |
| 38D | 3Bc | Industrial waste heat | 90-200 |
| 39A | 3Bc | Solar energy | NR |
| 39B | 3Bc | Solar energy | 100-400 |
| 39C | 3Bc | Solar energy | 150-350 |
| 39D | 3Bc | Solar energy | 200-300 |
| 40A | 3Bc | Geothermal water | NR |
| 40B | 3Bc | Geothermal water | 50-350 |
| 40C | 3Bc | Geothermal water | 100-300 |
| 40D | 3Bc | Geothermal water | 125-275 |
| 41A | 3Bc | Low pressure steam | NR |
| 41B | 3Bc | Low pressure steam | =<121 |
| 41C | 3Bc | Low pressure steam | =<115 |
| 41D | 3Bc | Low pressure steam | =<110 |
| 42A | 3Bc | distributed power generation equipment using fuel cells | NR |
| 42B | 3Bc | distributed power generation equipment using fuel cells | 60-200 |
| 42C | 3Bc | distributed power generation equipment using fuel cells | 80-200 |
| 42D | 3Bc | distributed power generation equipment using fuel cells | 100-200 |
| 43A | 3Bc | Exhaust gas from internaI combustion engine | NR |
| 43B | 3Bc | Exhaust gas from internaI combustion engine | =<500 |
| 43C | 3Bc | Exhaust gas from internaI combustion engine | =<450 |
| 43D | 3Bc | Exhaust gas from internaI combustion engine | =<400 |
| 44A | 4A1 | Industrial waste heat | NR |
| 44B | 4A1 | Industrial waste heat | 90-800 |
| 44C | 4A1 | Industrial waste heat | 90-400 |
| 44D | 4A1 | Industrial waste heat | 90-200 |
| 45A | 4A1 | Solar energy | NR |
| 45B | 4A1 | Solar energy | 100-400 |
| 45C | 4A1 | Solar energy | 150-350 |
| 45D | 4A1 | Solar energy | 200-300 |
| 46A | 4A1 | Geothermal water | NR |
| 46B | 4A1 | Geothermal water | 50-350 |
| 46C | 4A1 | Geothermal water | 100-300 |
| 46D | 4A1 | Geothermal water | 125-275 |
| 47A | 4A1 | Low pressure steam | NR |
| 47B | 4A1 | Low pressure steam | =<121 |
| 47C | 4A1 | Low pressure steam | =<115 |
| 47D | 4A1 | Low pressure steam | =<110 |
| 48A | 4A1 | distributed power generation equipment using fuel cells | NR |
| 48B | 4A1 | distributed power generation equipment using fuel cells | 60-200 |
| 48C | 4A1 | distributed power generation equipment using fuel cells | 80-200 |
| 48D | 4A1 | distributed power generation equipment using fuel cells | 100-200 |
| 49A | 4A1 | Exhaust gas from internaI combustion engine | NR |
| 49B | 4A1 | Exhaust gas from internaI combustion engine | =<500 |
| 49C | 4A1 | Exhaust gas from internaI combustion engine | =<450 |
| 49D | 4A1 | Exhaust gas from internaI combustion engine | =<400 |
| 50A | 4A2 | Industrial waste heat | NR |
| 50B | 4A2 | Industrial waste heat | 90-800 |
| 50C | 4A2 | Industrial waste heat | 90-400 |
| 50D | 4A2 | Industrial waste heat | 90-200 |
| 51A | 4A2 | Solar energy | NR |
| 51B | 4A2 | Solar energy | 100-400 |
| 51C | 4A2 | Solar energy | 150-350 |
| 51D | 4A2 | Solar energy | 200-300 |
| 52A | 4A2 | Geothermal water | NR |
| 52B | 4A2 | Geothermal water | 50-350 |
| 52C | 4A2 | Geothermal water | 100-300 |
| 52D | 4A2 | Geothermal water | 125-275 |
| 53A | 4A2 | Low pressure steam | NR |
| 53B | 4A2 | Low pressure steam | =<121 |
| 53C | 4A2 | Low pressure steam | =<115 |
| 53D | 4A2 | Low pressure steam | =<110 |
| 54A | 4A2 | distributed power generation equipment using fuel cells | NR |
| 54B | 4A2 | distributed power generation equipment using fuel cells | 60-200 |
| 54C | 4A2 | distributed power generation equipment using fuel cells | 80-200 |
| 54D | 4A2 | distributed power generation equipment using fuel cells | 100-200 |
| 55A | 4A2 | Exhaust gas from internaI combustion engine | NR |
| 55B | 4A2 | Exhaust gas from internaI combustion engine | =<500 |
| 55C | 4A2 | Exhaust gas from internaI combustion engine | =<450 |
| 55D | 4A2 | Exhaust gas from internaI combustion engine | =<400 |
| 56A | 4A3 | Industrial waste heat | NR |
| 56B | 4A3 | Industrial waste heat | 90-800 |
| 56C | 4A3 | Industrial waste heat | 90-400 |
| 56D | 4A3 | Industrial waste heat | 90-200 |
| 57A | 4A3 | Solar energy | NR |
| 57B | 4A3 | Solar energy | 100-400 |
| 57C | 4A3 | Solar energy | 150-350 |
| 57D | 4A3 | Solar energy | 200-300 |
| 58A | 4A3 | Geothermal water | NR |
| 58B | 4A3 | Geothermal water | 50-350 |
| 58C | 4A3 | Geothermal water | 100-300 |
| 58D | 4A3 | Geothermal water | 125-275 |
| 59A | 4A3 | Low pressure steam | NR |
| 59B | 4A3 | Low pressure steam | =<121 |
| 59C | 4A3 | Low pressure steam | =<115 |
| 59D | 4A3 | Low pressure steam | =<110 |
| 60A | 4A3 | distributed power generation equipment using fuel cells | NR |
| 60B | 4A3 | distributed power generation equipment using fuel cells | 60-200 |
| 60C | 4A3 | distributed power generation equipment using fuel cells | 80-200 |
| 60D | 4A3 | distributed power generation equipment using fuel cells | 100-200 |
| 61A | 4A3 | Exhaust gas from internaI combustion engine | NR |
| 61B | 4A3 | Exhaust gas from internaI combustion engine | =<500 |
| 61C | 4A3 | Exhaust gas from internaI combustion engine | =<450 |
| 61D | 4A3 | Exhaust gas from internaI combustion engine | =<400 |
| 62A | 4B1 | Industrial waste heat | NR |
| 62B | 4B1 | Industrial waste heat | 90-800 |
| 62C | 4B1 | Industrial waste heat | 90-400 |
| 62D | 4B1 | Industrial waste heat | 90-200 |
| 63A | 4B1 | Solar energy | NR |
| 63B | 4B1 | Solar energy | 100-400 |
| 63C | 4B1 | Solar energy | 150-350 |
| 63D | 4B1 | Solar energy | 200-300 |
| 64A | 4B1 | Geothermal water | NR |
| 64B | 4B1 | Geothermal water | 50-350 |
| 64C | 4B1 | Geothermal water | 100-300 |
| 64D | 4B1 | Geothermal water | 125-275 |
| 65A | 4B1 | Low pressure steam | NR |
| 65B | 4B1 | Low pressure steam | =<121 |
| 65C | 4B1 | Low pressure steam | =<115 |
| 65D | 4B1 | Low pressure steam | =<110 |
| 66A | 4B1 | distributed power generation equipment using fuel cells | NR |
| 66B | 4B1 | distributed power generation equipment using fuel cells | 60-200 |
| 66C | 4B1 | distributed power generation equipment using fuel cells | 80-200 |
| 66D | 4B1 | distributed power generation equipment using fuel cells | 100-200 |
| 67A | 4B1 | Exhaust gas from internaI combustion engine | NR |
| 67B | 4B1 | Exhaust gas from internaI combustion engine | =<500 |
| 67C | 4B1 | Exhaust gas from internaI combustion engine | =<450 |
| 67D | 4B1 | Exhaust gas from internaI combustion engine | =<400 |
| 68A | 4B2 | Industrial waste heat | NR |
| 68B | 4B2 | Industrial waste heat | 90-800 |
| 68C | 4B2 | Industrial waste heat | 90-400 |
| 68D | 4B2 | Industrial waste heat | 90-200 |
| 69A | 4B2 | Solar energy | NR |
| 69B | 4B2 | Solar energy | 100-400 |
| 69C | 4B2 | Solar energy | 150-350 |
| 69D | 4B2 | Solar energy | 200-300 |
| 70A | 4B2 | Geothermal water | NR |
| 70B | 4B2 | Geothermal water | 50-350 |
| 70C | 4B2 | Geothermal water | 100-300 |
| 70D | 4B2 | Geothermal water | 125-275 |
| 71A | 4B2 | Low pressure steam | NR |
| 71B | 4B2 | Low pressure steam | =<121 |
| 71C | 4B2 | Low pressure steam | =<115 |
| 71D | 4B2 | Low pressure steam | =<110 |
| 72A | 4B2 | distributed power generation equipment using fuel cells | NR |
| 72B | 4B2 | distributed power generation equipment using fuel cells | 60-200 |
| 72C | 4B2 | distributed power generation equipment using fuel cells | 80-200 |
| 72D | 4B2 | distributed power generation equipment using fuel cells | 100-200 |
| 73A | 4B2 | Exhaust gas from internaI combustion engine | NR |
| 73B | 4B2 | Exhaust gas from internaI combustion engine | =<500 |
| 73C | 4B2 | Exhaust gas from internaI combustion engine | =<450 |
| 73D | 4B2 | Exhaust gas from internaI combustion engine | =<400 |
| 74A | 4B3 | Industrial waste heat | NR |
| 74B | 4B3 | Industrial waste heat | 90-800 |
| 74C | 4B3 | Industrial waste heat | 90-400 |
| 74D | 4B3 | Industrial waste heat | 90-200 |
| 75A | 4B3 | Solar energy | NR |
| 75B | 4B3 | Solar energy | 100-400 |
| 75C | 4B3 | Solar energy | 150-350 |
| 75D | 4B3 | Solar energy | 200-300 |
| 76A | 4B3 | Geothermal water | NR |
| 76B | 4B3 | Geothermal water | 50-350 |
| 76C | 4B3 | Geothermal water | 100-300 |
| 76D | 4B3 | Geothermal water | 125-275 |
| 77A | 4B3 | Low pressure steam | NR |
| 77B | 4B3 | Low pressure steam | =<121 |
| 77C | 4B3 | Low pressure steam | =<115 |
| 77D | 4B3 | Low pressure steam | =<110 |
| 78A | 4B3 | distributed power generation equipment using fuel cells | NR |
| 78B | 4B3 | distributed power generation equipment using fuel cells | 60-200 |
| 78C | 4B3 | distributed power generation equipment using fuel cells | 80-200 |
| 78D | 4B3 | distributed power generation equipment using fuel cells | 100-200 |
| 79A | 4B3 | Exhaust gas from internaI combustion engine | NR |
| 79B | 4B3 | Exhaust gas from internaI combustion engine | =<500 |
| 79C | 4B3 | Exhaust gas from internaI combustion engine | =<450 |
| 79D | 4B3 | Exhaust gas from internaI combustion engine | =<400 |
| 80A | 4C1 | Industrial waste heat | NR |
| 80B | 4C1 | Industrial waste heat | 90-800 |
| 80C | 4C1 | Industrial waste heat | 90-400 |
| 80D | 4C1 | Industrial waste heat | 90-200 |
| 81A | 4C1 | Solar energy | NR |
| 81B | 4C1 | Solar energy | 100-400 |
| 81C | 4C1 | Solar energy | 150-350 |
| 81D | 4C1 | Solar energy | 200-300 |
| 82A | 4C1 | Geothermal water | NR |
| 82B | 4C1 | Geothermal water | 50-350 |
| 82C | 4C1 | Geothermal water | 100-300 |
| 83D | 4C1 | Geothermal water | 125-275 |
| 84A | 4C1 | Low pressure steam | NR |
| 84B | 4C1 | Low pressure steam | =<121 |
| 84C | 4C1 | Low pressure steam | =<115 |
| 84D | 4C1 | Low pressure steam | =<110 |
| 85A | 4C1 | distributed power generation equipment using fuel cells | NR |
| 85B | 4C1 | distributed power generation equipment using fuel cells | 60-200 |
| 85C | 4C1 | distributed power generation equipment using fuel cells | 80-200 |
| 85D | 4C1 | distributed power generation equipment using fuel cells | 100-200 |
| 86A | 4C1 | Exhaust gas from internaI combustion engine | NR |
| 86A | 4C1 | Exhaust gas from internaI combustion engine | =<500 |
| 86B | 4C1 | Exhaust gas from internaI combustion engine | =<450 |
| 86C | 4C1 | Exhaust gas from internaI combustion engine | =<400 |
| 86D | 4C1 | Exhaust gas from internaI combustion engine | NR |
| 87A | 4C2 | Industrial waste heat | NR |
| 87B | 4C2 | Industrial waste heat | 90-800 |
| 87C | 4C2 | Industrial waste heat | 90-400 |
| 87D | 4C2 | Industrial waste heat | 90-200 |
| 88A | 4C2 | Solar energy | NR |
| 88B | 4C2 | Solar energy | 100-400 |
| 88C | 4C2 | Solar energy | 150-350 |
| 88D | 4C2 | Solar energy | 200-300 |
| 89A | 4C2 | Geothermal water | NR |
| 89B | 4C2 | Geothermal water | 50-350 |
| 89C | 4C2 | Geothermal water | 100-300 |
| 89D | 4C2 | Geothermal water | 125-275 |
| 90A | 4C2 | Low pressure steam | NR |
| 90B | 4C2 | Low pressure steam | =<121 |
| 90C | 4C2 | Low pressure steam | =<115 |
| 90D | 4C2 | Low pressure steam | =<110 |
| 91A | 4C2 | distributed power generation equipment using fuel cells | NR |
| 91B | 4C2 | distributed power generation equipment using fuel cells | 60-200 |
| 91C | 4C2 | distributed power generation equipment using fuel cells | 80-200 |
| 91D | 4C2 | distributed power generation equipment using fuel cells | 100-200 |
| 92A | 4C2 | Exhaust gas from internaI combustion engine | NR |
| 92B | 4C2 | Exhaust gas from internaI combustion engine | =<500 |
| 92C | 4C2 | Exhaust gas from internaI combustion engine | =<450 |
| 92D | 4C2 | Exhaust gas from internaI combustion engine | =<400 |
| 93A | 4C3 | Industrial waste heat | NR |
| 93B | 4C3 | Industrial waste heat | 90-800 |
| 93C | 4C3 | Industrial waste heat | 90-400 |
| 93D | 4C3 | Industrial waste heat | 90-200 |
| 93A | 4C3 | Solar energy | NR |
| 94B | 4C3 | Solar energy | 100-400 |
| 94C | 4C3 | Solar energy | 150-350 |
| 94D | 4C3 | Solar energy | 200-300 |
| 95A | 4C3 | Geothermal water | NR |
| 95B | 4C3 | Geothermal water | 50-350 |
| 95B | 4C3 | Geothermal water | 100-300 |
| 95C | 4C3 | Geothermal water | 125-275 |
| 96A | 4C3 | Low pressure steam | NR |
| 96B | 4C3 | Low pressure steam | =<121 |
| 96C | 4C3 | Low pressure steam | =<115 |
| 96D | 4C3 | Low pressure steam | =<110 |
| 97A | 4C3 | distributed power generation equipment using fuel cells | NR |
| 97B | 4C3 | distributed power generation equipment using fuel cells | 60-200 |
| 97C | 4C3 | distributed power generation equipment using fuel cells | 80-200 |
| 97D | 4C3 | distributed power generation equipment using fuel cells | 100-200 |
| 98A | 4C3 | Exhaust gas from internaI combustion engine | NR |
| 98B | 4C3 | Exhaust gas from internaI combustion engine | =<500 |
| 98C | 4C3 | Exhaust gas from internaI combustion engine | =<450 |
| 98D | 4C3 | Exhaust gas from internaI combustion engine | =<400 |
| 99A | 5A | Industrial waste heat | NR |
| 99B | 5A | Industrial waste heat | 90-800 |
| 99C | 5A | Industrial waste heat | 90-400 |
| 99D | 5A | Industrial waste heat | 90-200 |
| 100A | 5A | Solar energy | NR |
| 100B | 5A | Solar energy | 100-400 |
| 100C | 5A | Solar energy | 150-350 |
| 100D | 5A | Solar energy | 200-300 |
| 101A | 5A | Geothermal water | NR |
| 101B | 5A | Geothermal water | 50-350 |
| 101C | 5A | Geothermal water | 100-300 |
| 101D | 5A | Geothermal water | 125-275 |
| 102A | 5A | Low pressure steam | NR |
| 102B | 5A | Low pressure steam | =<121 |
| 102C | 5A | Low pressure steam | =<115 |
| 102D | 5A | Low pressure steam | =<110 |
| 103A | 5A | distributed power generation equipment using fuel cells | NR |
| 103B | 5A | distributed power generation equipment using fuel cells | 60-200 |
| 103C | 5A | distributed power generation equipment using fuel cells | 80-200 |
| 103D | 5A | distributed power generation equipment using fuel cells | 100-200 |
| 104A | 5A | Exhaust gas from internaI combustion engine | NR |
| 104B | 5A | Exhaust gas from internaI combustion engine | =<500 |
| 104C | 5A | Exhaust gas from internaI combustion engine | =<450 |
| 104D | 5A | Exhaust gas from internaI combustion engine | =<400 |
| 105A | 5B | Industrial waste heat | NR |
| 105B | 5B | Industrial waste heat | 90-800 |
| 105C | 5B | Industrial waste heat | 90-400 |
| 105D | 5B | Industrial waste heat | 90-200 |
| 106A | 5B | Solar energy | NR |
| 106B | 5B | Solar energy | 100-400 |
| 106C | 5B | Solar energy | 150-350 |
| 106D | 5B | Solar energy | 200-300 |
| 107A | 5B | Geothermal water | NR |
| 107B | 5B | Geothermal water | 50-350 |
| 107C | 5B | Geothermal water | 100-300 |
| 107D | 5B | Geothermal water | 125-275 |
| 108A | 5B | Low pressure steam | NR |
| 108B | 5B | Low pressure steam | =<121 |
| 108C | 5B | Low pressure steam | =<115 |
| 108D | 5B | Low pressure steam | =<110 |
| 109A | 5B | distributed power generation equipment using fuel cells | NR |
| 109B | 5B | distributed power generation equipment using fuel cells | 60-200 |
| 109C | 5B | distributed power generation equipment using fuel cells | 80-200 |
| 109D | 5B | distributed power generation equipment using fuel cells | 100-200 |
| 110A | 5B | Exhaust gas from internaI combustion engine | NR |
| 110B | 5B | Exhaust gas from internaI combustion engine | =<500 |
| 110C | 5B | Exhaust gas from internaI combustion engine | =<450 |
| 110D | 5B | Exhaust gas from internaI combustion engine | =<400 |
| 111A | 5C | Industrial waste heat | NR |
| 111B | 5C | Industrial waste heat | 90-800 |
| 111C | 5C | Industrial waste heat | 90-400 |
| 111D | 5C | Industrial waste heat | 90-200 |
| 112A | 5C | Solar energy | NR |
| 112B | 5C | Solar energy | 100-400 |
| 112C | 5C | Solar energy | 150-350 |
| 112D | 5C | Solar energy | 200-300 |
| 113A | 5C | Geothermal water | NR |
| 113B | 5C | Geothermal water | 50-350 |
| 113C | 5C | Geothermal water | 100-300 |
| 113D | 5C | Geothermal water | 125-275 |
| 114A | 5C | Low pressure steam | NR |
| 114B | 5C | Low pressure steam | =<121 |
| 114C | 5C | Low pressure steam | =<115 |
| 114D | 5C | Low pressure steam | =<110 |
| 115A | 5C | distributed power generation equipment using fuel cells | NR |
| 115B | 5C | distributed power generation equipment using fuel cells | 60-200 |
| 115C | 5C | distributed power generation equipment using fuel cells | 80-200 |
| 115D | 5C | distributed power generation equipment using fuel cells | 100-200 |
| 116A | 5C | Exhaust gas from internaI combustion engine | NR |
| 116B | 5C | Exhaust gas from internaI combustion engine | =<500 |
| 116C | 5C | Exhaust gas from internaI combustion engine | =<450 |
| 116D | 5C | Exhaust gas from internaI combustion engine | =<400 |
| 117A | 5D | Industrial waste heat | NR |
| 117B | 5D | Industrial waste heat | 90-800 |
| 117C | 5D | Industrial waste heat | 90-400 |
| 117D | 5D | Industrial waste heat | 90-200 |
| 118A | 5D | Solar energy | NR |
| 118B | 5D | Solar energy | 100-400 |
| 118C | 5D | Solar energy | 150-350 |
| 118D | 5D | Solar energy | 200-300 |
| 119A | 5D | Geothermal water | NR |
| 119B | 5D | Geothermal water | 50-350 |
| 119C | 5D | Geothermal water | 100-300 |
| 119D | 5D | Geothermal water | 125-275 |
| 120A | 5D | Low pressure steam | NR |
| 120B | 5D | Low pressure steam | =<121 |
| 120C | 5D | Low pressure steam | =<115 |
| 120D | 5D | Low pressure steam | =<110 |
| 121A | 5D | distributed power generation equipment using fuel cells | NR |
| 121B | 5D | distributed power generation equipment using fuel cells | 60-200 |
| 121C | 5D | distributed power generation equipment using fuel cells | 80-200 |
| 121D | 5D | distributed power generation equipment using fuel cells | 100-200 |
| 122A | 5D | Exhaust gas from internaI combustion engine | NR |
| 122B | 5D | Exhaust gas from internaI combustion engine | =<500 |
| 122C | 5D | Exhaust gas from internaI combustion engine | =<450 |
| 122D | 5D | Exhaust gas from internaI combustion engine | =<400 |
| 123A | HTC1-1 | Industrial waste heat | NR |
| 123B | HTC1-1 | Industrial waste heat | 90-800 |
| 123C | HTC1-1 | Industrial waste heat | 90-400 |
| 123D | HTC1-1 | Industrial waste heat | 90-200 |
| 124A | HTC1-1 | Solar energy | NR |
| 124B | HTC1-1 | Solar energy | 100-400 |
| 124C | HTC1-1 | Solar energy | 150-350 |
| 124D | HTC1-1 | Solar energy | 200-300 |
| 125A | HTC1-1 | Geothermal water | NR |
| 125B | HTC1-1 | Geothermal water | 50-350 |
| 125C | HTC1-1 | Geothermal water | 100-300 |
| 125D | HTC1-1 | Geothermal water | 125-275 |
| 126A | HTC1-1 | Low pressure steam | NR |
| 126B | HTC1-1 | Low pressure steam | =<121 |
| 126C | HTC1-1 | Low pressure steam | =<115 |
| 126D | HTC1-1 | Low pressure steam | =<110 |
| 127A | HTC1-1 | distributed power generation equipment using fuel cells | NR |
| 127B | HTC1-1 | distributed power generation equipment using fuel cells | 60-200 |
| 127C | HTC1-1 | distributed power generation equipment using fuel cells | 80-200 |
| 127D | HTC1-1 | distributed power generation equipment using fuel cells | 100-200 |
| 128A | HTC1-1 | Exhaust gas from internaI combustion engine | NR |
| 128B | HTC1-1 | Exhaust gas from internaI combustion engine | =<500 |
| 128C | HTC1-1 | Exhaust gas from internaI combustion engine | =<450 |
| 128D | HTC1-1 | Exhaust gas from internaI combustion engine | =<400 |
| 129A | HTC1-2 | Industrial waste heat | NR |
| 129B | HTC1-2 | Industrial waste heat | 90-800 |
| 129C | HTC1-2 | Industrial waste heat | 90-400 |
| 129D | HTC1-2 | Industrial waste heat | 90-200 |
| 130A | HTC1-2 | Solar energy | NR |
| 130B | HTC1-2 | Solar energy | 100-400 |
| 130C | HTC1-2 | Solar energy | 150-350 |
| 130D | HTC1-2 | Solar energy | 200-300 |
| 131A | HTC1-2 | Geothermal water | NR |
| 131B | HTC1-2 | Geothermal water | 50-350 |
| 131C | HTC1-2 | Geothermal water | 100-300 |
| 131D | HTC1-2 | Geothermal water | 125-275 |
| 132A | HTC1-2 | Low pressure steam | NR |
| 132B | HTC1-2 | Low pressure steam | =<121 |
| 132C | HTC1-2 | Low pressure steam | =<115 |
| 132D | HTC1-2 | Low pressure steam | =<110 |
| 133A | HTC1-2 | distributed power generation equipment using fuel cells | NR |
| 133B | HTC1-2 | distributed power generation equipment using fuel cells | 60-200 |
| 133C | HTC1-2 | distributed power generation equipment using fuel cells | 80-200 |
| 133D | HTC1-2 | distributed power generation equipment using fuel cells | 100-200 |
| 134A | HTC1-3 | Industrial waste heat | NR |
| 134B | HTC1-3 | Industrial waste heat | 90-800 |
| 134C | HTC1-3 | Industrial waste heat | 90-400 |
| 134D | HTC1-3 | Industrial waste heat | 90-200 |
| 135A | HTC1-3 | Solar energy | NR |
| 135B | HTC1-3 | Solar energy | 100-400 |
| 135C | HTC1-3 | Solar energy | 150-350 |
| 135D | HTC1-3 | Solar energy | 200-300 |
| 136A | HTC1-3 | Geothermal water | NR |
| 136B | HTC1-3 | Geothermal water | 50-350 |
| 136C | HTC1-3 | Geothermal water | 100-300 |
| 136D | HTC1-3 | Geothermal water | 125-275 |
| 137A | HTC1-3 | Low pressure steam | NR |
| 138B | HTC1-3 | Low pressure steam | =<121 |
| 138C | HTC1-3 | Low pressure steam | =<115 |
| 139D | HTC1-3 | Low pressure steam | =<110 |
| 140A | HTC1-3 | distributed power generation equipment using fuel cells | NR |
| 140B | HTC1-3 | distributed power generation equipment using fuel cells | 60-200 |
| 140C | HTC1-3 | distributed power generation equipment using fuel cells | 80-200 |
| 140D | HTC1-3 | distributed power generation equipment using fuel cells | 100-200 |
| 141A | HTC1-3 | Exhaust gas from internaI combustion engine | NR |
| 141B | HTC1-3 | Exhaust gas from internaI combustion engine | =<500 |
| 141C | HTC1-3 | Exhaust gas from internaI combustion engine | =<450 |
| 141D | HTC1-3 | Exhaust gas from internaI combustion engine | =<400 |
| 142A | HTC1-3 | Industrial waste heat | NR |
| 142B | HTC1-3 | Industrial waste heat | 90-800 |
| 142C | HTC1-3 | Industrial waste heat | 90-400 |
| 142D | HTC1-3 | Industrial waste heat | 90-200 |
| 143A | HTC1-4 | Solar energy | NR |
| 143B | HTC1-4 | Solar energy | 100-400 |
| 143C | HTC1-4 | Solar energy | 150-350 |
| 143D | HTC1-4 | Solar energy | 200-300 |
| 144A | HTC1-4 | Geothermal water | NR |
| 144B | HTC1-4 | Geothermal water | 50-350 |
| 144C | HTC1-4 | Geothermal water | 100-300 |
| 144D | HTC1-4 | Geothermal water | 125-275 |
| 144A | HTC1-4 | Low pressure steam | NR |
| 144B | HTC1-4 | Low pressure steam | =<121 |
| 145C | HTC1-4 | Low pressure steam | =<115 |
| 145D | HTC1-4 | Low pressure steam | =<110 |
| 146A | HTC1-4 | distributed power generation equipment using fuel cells | NR |
| 146B | HTC1-4 | distributed power generation equipment using fuel cells | 60-200 |
| 146C | HTC1-4 | distributed power generation equipment using fuel cells | 80-200 |
| 146D | HTC1-4 | distributed power generation equipment using fuel cells | 100-200 |
| 147A | HTC1-4 | Exhaust gas from internaI combustion engine | NR |
| 147B | HTC1-4 | Exhaust gas from internaI combustion engine | =<500 |
| 147C | HTC1-4 | Exhaust gas from internaI combustion engine | =<450 |
| 147D | HTC1-4 | Exhaust gas from internaI combustion engine | =<400 |
| 148A | HTC1-5 | Industrial waste heat | NR |
| 148B | HTC1-5 | Industrial waste heat | 90-800 |
| 148C | HTC1-5 | Industrial waste heat | 90-400 |
| 148D | HTC1-5 | Industrial waste heat | 90-200 |
| 149A | HTC1-5 | Solar energy | NR |
| 149B | HTC1-5 | Solar energy | 100-400 |
| 149C | HTC1-5 | Solar energy | 150-350 |
| 149D | HTC1-5 | Solar energy | 200-300 |
| 150A | HTC1-5 | Geothermal water | NR |
| 150B | HTC1-5 | Geothermal water | 50-350 |
| 150C | HTC1-5 | Geothermal water | 100-300 |
| 150D | HTC1-5 | Geothermal water | 125-275 |
| 151A | HTC1-5 | Low pressure steam | NR |
| 151B | HTC1-5 | Low pressure steam | =<121 |
| 151C | HTC1-5 | Low pressure steam | =<115 |
| 151D | HTC1-5 | Low pressure steam | =<110 |
| 152A | HTC1-5 | distributed power generation equipment using fuel cells | NR |
| 152B | HTC1-5 | distributed power generation equipment using fuel cells | 60-200 |
| 152C | HTC1-5 | distributed power generation equipment using fuel cells | 80-200 |
| 152D | HTC1-5 | distributed power generation equipment using fuel cells | 100-200 |
| 153A | HTC1-5 | Exhaust gas from internaI combustion engine | NR |
| 153B | HTC1-5 | Exhaust gas from internaI combustion engine | =<500 |
| 153C | HTC1-5 | Exhaust gas from internaI combustion engine | =<450 |
| 153D | HTC1-5 | Exhaust gas from internaI combustion engine | =<400 |
| 154A | HTC1-6 | Industrial waste heat | NR |
| 154B | HTC1-6 | Industrial waste heat | 90-800 |
| 154C | HTC1-6 | Industrial waste heat | 90-400 |
| 155D | HTC1-6 | Industrial waste heat | 90-200 |
| 156A | HTC1-6 | Solar energy | NR |
| 156B | HTC1-6 | Solar energy | 100-400 |
| 156C | HTC1-6 | Solar energy | 150-350 |
| 156D | HTC1-6 | Solar energy | 200-300 |
| 157A | HTC1-6 | Geothermal water | NR |
| 157B | HTC1-6 | Geothermal water | 50-350 |
| 157C | HTC1-6 | Geothermal water | 100-300 |
| 157D | HTC1-6 | Geothermal water | 125-275 |
| 158A | HTC1-6 | Low pressure steam | NR |
| 158B | HTC1-6 | Low pressure steam | =<121 |
| 158C | HTC1-6 | Low pressure steam | =<115 |
| 158D | HTC1-6 | Low pressure steam | =<110 |
| 159A | HTC1-6 | distributed power generation equipment using fuel cells | NR |
| 159B | HTC1-6 | distributed power generation equipment using fuel cells | 60-200 |
| 159C | HTC1-6 | distributed power generation equipment using fuel cells | 80-200 |
| 159D | HTC1-6 | distributed power generation equipment using fuel cells | 100-200 |
| 160A | HTC1-6 | Exhaust gas from internaI combustion engine | NR |
| 160B | HTC1-6 | Exhaust gas from internaI combustion engine | =<500 |
| 160C | HTC1-6 | Exhaust gas from internaI combustion engine | =<450 |
| 160D | HTC1-6 | Exhaust gas from internaI combustion engine | =<400 |
| 161A | HTC1-7 | Industrial waste heat | NR |
| 161B | HTC1-7 | Industrial waste heat | 90-800 |
| 161C | HTC1-7 | Industrial waste heat | 90-400 |
| 161D | HTC1-7 | Industrial waste heat | 90-200 |
| 162A | HTC1-7 | Solar energy | NR |
| 162B | HTC1-7 | Solar energy | 100-400 |
| 162C | HTC1-7 | Solar energy | 150-350 |
| 162D | HTC1-7 | Solar energy | 200-300 |
| 163A | HTC1-7 | Geothermal water | NR |
| 163B | HTC1-7 | Geothermal water | 50-350 |
| 163C | HTC1-7 | Geothermal water | 100-300 |
| 163D | HTC1-7 | Geothermal water | 125-275 |
| 164A | HTC1-7 | Low pressure steam | NR |
| 164B | HTC1-7 | Low pressure steam | =<121 |
| 164C | HTC1-7 | Low pressure steam | =<115 |
| 164D | HTC1-7 | Low pressure steam | =<110 |
| 165A | HTC1-7 | distributed power generation equipment using fuel cells | NR |
| 165B | HTC1-7 | distributed power generation equipment using fuel cells | 60-200 |
| 165C | HTC1-7 | distributed power generation equipment using fuel cells | 80-200 |
| 165D | HTC1-7 | distributed power generation equipment using fuel cells | 100-200 |
| 166A | HTC1-7 | Exhaust gas from internaI combustion engine | NR |
| 166B | HTC1-7 | Exhaust gas from internaI combustion engine | =<500 |
| 166C | HTC1-7 | Exhaust gas from internaI combustion engine | =<450 |
| 166D | HTC1-7 | Exhaust gas from internaI combustion engine | =<400 |
| 167A | HTC2-1 | Industrial waste heat | NR |
| 167B | HTC2-1 | Industrial waste heat | 90-800 |
| 167C | HTC2-1 | Industrial waste heat | 90-400 |
| 167D | HTC2-1 | Industrial waste heat | 90-200 |
| 168A | HTC2-1 | Solar energy | NR |
| 168B | HTC2-1 | Solar energy | 100-400 |
| 168C | HTC2-1 | Solar energy | 150-350 |
| 168D | HTC2-1 | Solar energy | 200-300 |
| 169A | HTC2-1 | Geothermal water | NR |
| 169B | HTC2-1 | Geothermal water | 50-350 |
| 169C | HTC2-1 | Geothermal water | 100-300 |
| 169D | HTC2-1 | Geothermal water | 125-275 |
| 170A | HTC2-1 | Low pressure steam | NR |
| 170B | HTC2-1 | Low pressure steam | =<121 |
| 170C | HTC2-1 | Low pressure steam | =<115 |
| 170D | HTC2-1 | Low pressure steam | =<110 |
| 171A | HTC2-1 | distributed power generation equipment using fuel cells | NR |
| 171B | HTC2-1 | distributed power generation equipment using fuel cells | 60-200 |
| 171C | HTC2-1 | distributed power generation equipment using fuel cells | 80-200 |
| 171D | HTC2-1 | distributed power generation equipment using fuel cells | 100-200 |
| 172A | HTC2-1 | Exhaust gas from internaI combustion engine | NR |
| 172B | HTC2-1 | Exhaust gas from internaI combustion engine | =<500 |
| 172C | HTC2-1 | Exhaust gas from internaI combustion engine | =<450 |
| 172D | HTC2-1 | Exhaust gas from internaI combustion engine | =<400 |
| 173A | HTC2-2 | Industrial waste heat | NR |
| 173B | HTC2-2 | Industrial waste heat | 90-800 |
| 173C | HTC2-2 | Industrial waste heat | 90-400 |
| 173D | HTC2-2 | Industrial waste heat | 90-200 |
| 174A | HTC2-2 | Solar energy | NR |
| 174B | HTC2-2 | Solar energy | 100-400 |
| 174C | HTC2-2 | Solar energy | 150-350 |
| 174D | HTC2-2 | Solar energy | 200-300 |
| 175A | HTC2-2 | Geothermal water | NR |
| 175B | HTC2-2 | Geothermal water | 50-350 |
| 175C | HTC2-2 | Geothermal water | 100-300 |
| 175D | HTC2-2 | Geothermal water | 125-275 |
| 176A | HTC2-2 | Low pressure steam | NR |
| 176B | HTC2-2 | Low pressure steam | =<121 |
| 176C | HTC2-2 | Low pressure steam | =<115 |
| 176D | HTC2-2 | Low pressure steam | =<110 |
| 177A | HTC2-2 | distributed power generation equipment using fuel cells | NR |
| 177B | HTC2-2 | distributed power generation equipment using fuel cells | 60-200 |
| 177C | HTC2-2 | distributed power generation equipment using fuel cells | 80-200 |
| 177D | HTC2-2 | distributed power generation equipment using fuel cells | 100-200 |
| 178A | HTC2-2 | Exhaust gas from internaI combustion engine | NR |
| 178B | HTC2-2 | Exhaust gas from internaI combustion engine | =<500 |
| 178C | HTC2-2 | Exhaust gas from internaI combustion engine | =<450 |
| 178D | HTC2-2 | Exhaust gas from internaI combustion engine | =<400 |
| 179A | HTC2-3 | Industrial waste heat | NR |
| 179B | HTC2-3 | Industrial waste heat | 90-800 |
| 179C | HTC2-3 | Industrial waste heat | 90-400 |
| 179D | HTC2-3 | Industrial waste heat | 90-200 |
| 180A | HTC2-3 | Solar energy | NR |
| 180B | HTC2-3 | Solar energy | 100-400 |
| 180C | HTC2-3 | Solar energy | 150-350 |
| 180D | HTC2-3 | Solar energy | 200-300 |
| 181A | HTC2-3 | Geothermal water | NR |
| 181B | HTC2-3 | Geothermal water | 50-350 |
| 181C | HTC2-3 | Geothermal water | 100-300 |
| 181D | HTC2-3 | Geothermal water | 125-275 |
| 182A | HTC2-3 | Low pressure steam | NR |
| 182B | HTC2-3 | Low pressure steam | =<121 |
| 182C | HTC2-3 | Low pressure steam | =<115 |
| 183D | HTC2-3 | Low pressure steam | =<110 |
| 184A | HTC2-3 | distributed power generation equipment using fuel cells | NR |
| 184B | HTC2-3 | distributed power generation equipment using fuel cells | 60-200 |
| 184C | HTC2-3 | distributed power generation equipment using fuel cells | 80-200 |
| 184D | HTC2-3 | distributed power generation equipment using fuel cells | 100-200 |
| 185A | HTC2-3 | Exhaust gas from internaI combustion engine | NR |
| 85B | HTC2-3 | Exhaust gas from internaI combustion engine | =<500 |
| 185C | HTC2-3 | Exhaust gas from internaI combustion engine | =<450 |
| 185D | HTC2-3 | Exhaust gas from internaI combustion engine | =<400 |
| 186A | HTC2-4 | Industrial waste heat | NR |
| 186B | HTC2-4 | Industrial waste heat | 90-800 |
| 186C | HTC2-4 | Industrial waste heat | 90-400 |
| 186D | HTC2-4 | Industrial waste heat | 90-200 |
| 187A | HTC2-4 | Solar energy | NR |
| 187B | HTC2-4 | Solar energy | 100-400 |
| 187C | HTC2-4 | Solar energy | 150-350 |
| 187D | HTC2-4 | Solar energy | 200-300 |
| 188A | HTC2-4 | Geothermal water | NR |
| 188B | HTC2-4 | Geothermal water | 50-350 |
| 188C | HTC2-4 | Geothermal water | 100-300 |
| 188D | HTC2-4 | Geothermal water | 125-275 |
| 189A | HTC2-4 | Low pressure steam | NR |
| 189B | HTC2-4 | Low pressure steam | =<121 |
| 189C | HTC2-4 | Low pressure steam | =<115 |
| 189D | HTC2-4 | Low pressure steam | =<110 |
| 190A | HTC2-4 | distributed power generation equipment using fuel cells | NR |
| 190B | HTC2-4 | distributed power generation equipment using fuel cells | 60-200 |
| 190C | HTC2-4 | distributed power generation equipment using fuel cells | 80-200 |
| 190D | HTC2-4 | distributed power generation equipment using fuel cells | 100-200 |
| 191A | HTC2-4 | Exhaust gas from internaI combustion engine | NR |
| 191B | HTC2-4 | Exhaust gas from internaI combustion engine | =<500 |
| 191C | HTC2-4 | Exhaust gas from internaI combustion engine | =<450 |
| 191D | HTC2-4 | Exhaust gas from internaI combustion engine | =<400 |
| 192A | HTC2-5 | Industrial waste heat | NR |
| 192B | HTC2-5 | Industrial waste heat | 90-800 |
| 192C | HTC2-5 | Industrial waste heat | 90-400 |
| 192D | HTC2-5 | Industrial waste heat | 90-200 |
| 193A | HTC2-5 | Solar energy | NR |
| 193B | HTC2-5 | Solar energy | 100-400 |
| 193C | HTC2-5 | Solar energy | 150-350 |
| 193D | HTC2-5 | Solar energy | 200-300 |
| 194A | HTC2-5 | Geothermal water | NR |
| 194B | HTC2-5 | Geothermal water | 50-350 |
| 194C | HTC2-5 | Geothermal water | 100-300 |
| 194D | HTC2-5 | Geothermal water | 125-275 |
| 194A | HTC2-5 | Low pressure steam | NR |
| 195B | HTC2-5 | Low pressure steam | =<121 |
| 195C | HTC2-5 | Low pressure steam | =<115 |
| 195D | HTC2-5 | Low pressure steam | =<110 |
| 195A | HTC2-5 | distributed power generation equipment using fuel cells | NR |
| 196B | HTC2-5 | distributed power generation equipment using fuel cells | 60-200 |
| 196C | HTC2-5 | distributed power generation equipment using fuel cells | 80-200 |
| 196D | HTC2-5 | distributed power generation equipment using fuel cells | 100-200 |
| 197A | HTC2-5 | Exhaust gas from internaI combustion engine | NR |
| 197B | HTC2-5 | Exhaust gas from internaI combustion engine | =<500 |
| 197C | HTC2-5 | Exhaust gas from internaI combustion engine | =<450 |
| 197D | HTC2-5 | Exhaust gas from internaI combustion engine | =<400 |
| 198A | HTC2-6 | Industrial waste heat | NR |
| 198B | HTC2-6 | Industrial waste heat | 90-800 |
| 198C | HTC2-6 | Industrial waste heat | 90-400 |
| 198D | HTC2-6 | Industrial waste heat | 90-200 |
| 199A | HTC2-6 | Solar energy | NR |
| 199B | HTC2-6 | Solar energy | 90-800 |
| 199C | HTC2-6 | Solar energy | 90-400 |
| 199D | HTC2-6 | Solar energy | 90-200 |
| 200A | HTC2-6 | Geothermal water | NR |
| 200B | HTC2-6 | Geothermal water | 100-400 |
| 200C | HTC2-6 | Geothermal water | 150-350 |
| 200D | HTC2-6 | Geothermal water | 200-300 |
| 201A | HTC2-6 | Low pressure steam | NR |
| 201B | HTC2-6 | Low pressure steam | 50-350 |
| 201C | HTC2-6 | Low pressure steam | 100-300 |
| 201D | HTC2-6 | Low pressure steam | 125-275 |
| 202A | HTC2-6 | distributed power generation equipment using fuel cells | NR |
| 202B | HTC2-6 | distributed power generation equipment using fuel cells | =<121 |
| 202C | HTC2-6 | distributed power generation equipment using fuel cells | =<115 |
| 202D | HTC2-6 | distributed power generation equipment using fuel cells | =<110 |
| 203A | HTC2-6 | Exhaust gas from internaI combustion engine | NR |
| 203B | HTC2-6 | Exhaust gas from internaI combustion engine | 60-200 |
| 203C | HTC2-6 | Exhaust gas from internaI combustion engine | 80-200 |
| 203D | HTC2-6 | Exhaust gas from internaI combustion engine | 100-200 |
| 204A | HTC2-7 | Industrial waste heat | NR |
| 204B | HTC2-7 | Industrial waste heat | 90-800 |
| 204C | HTC2-7 | Industrial waste heat | 90-400 |
| 204D | HTC2-7 | Industrial waste heat | 90-200 |
| 205A | HTC2-7 | Solar energy | NR |
| 205B | HTC2-7 | Solar energy | 100-400 |
| 205C | HTC2-7 | Solar energy | 150-350 |
| 205D | HTC2-7 | Solar energy | 200-300 |
| 206A | HTC2-7 | Geothermal water | NR |
| 206B | HTC2-7 | Geothermal water | 50-350 |
| 206C | HTC2-7 | Geothermal water | 100-300 |
| 206D | HTC2-7 | Geothermal water | 125-275 |
| 207A | HTC2-7 | Low pressure steam | NR |
| 207B | HTC2-7 | Low pressure steam | =<121 |
| 207C | HTC2-7 | Low pressure steam | =<115 |
| 207D | HTC2-7 | Low pressure steam | =<110 |
| 208A | HTC2-7 | distributed power generation equipment using fuel cells | NR |
| 208B | HTC2-7 | distributed power generation equipment using fuel cells | 60-200 |
| 208C | HTC2-7 | distributed power generation equipment using fuel cells | 80-200 |
| 208D | HTC2-7 | distributed power generation equipment using fuel cells | 100-200 |
| 209A | HTC2-7 | Exhaust gas from internaI combustion engine | NR |
| 209B | HTC2-7 | Exhaust gas from internaI combustion engine | =<500 |
| 209C | HTC2-7 | Exhaust gas from internaI combustion engine | =<450 |
| 209D | HTC2-7 | Exhaust gas from internaI combustion engine | =<400 |
| 210A | HTC3-1 | Industrial waste heat | NR |
| 210B | HTC3-1 | Industrial waste heat | 90-800 |
| 210C | HTC3-1 | Industrial waste heat | 90-400 |
| 210D | HTC3-1 | Industrial waste heat | 90-200 |
| 211A | HTC3-1 | Solar energy | NR |
| 211B | HTC3-1 | Solar energy | 90-800 |
| 211C | HTC3-1 | Solar energy | 90-400 |
| 211D | HTC3-1 | Solar energy | 90-200 |
| 212A | HTC3-1 | Geothermal water | NR |
| 212B | HTC3-1 | Geothermal water | 100-400 |
| 212C | HTC3-1 | Geothermal water | 150-350 |
| 212D | HTC3-1 | Geothermal water | 200-300 |
| 213A | HTC3-1 | Low pressure steam | NR |
| 213B | HTC3-1 | Low pressure steam | 50-350 |
| 213C | HTC3-1 | Low pressure steam | 100-300 |
| 213D | HTC3-1 | Low pressure steam | 125-275 |
| 214A | HTC3-1 | distributed power generation equipment using fuel cells | NR |
| 214B | HTC3-1 | distributed power generation equipment using fuel cells | =<121 |
| 214C | HTC3-1 | distributed power generation equipment using fuel cells | =<115 |
| 214D | HTC3-1 | distributed power generation equipment using fuel cells | =<110 |
| 215A | HTC3-1 | Exhaust gas from internaI combustion engine | NR |
| 215B | HTC3-1 | Exhaust gas from internaI combustion engine | 60-200 |
| 215C | HTC3-1 | Exhaust gas from internaI combustion engine | 80-200 |
| 215D | HTC3-1 | Exhaust gas from internaI combustion engine | 100-200 |
| 216A | HTC3-2 | Industrial waste heat | NR |
| 216B | HTC3-2 | Industrial waste heat | 90-800 |
| 216C | HTC3-2 | Industrial waste heat | 90-400 |
| 216D | HTC3-2 | Industrial waste heat | 90-200 |
| 217A | HTC3-2 | Solar energy | NR |
| 217B | HTC3-2 | Solar energy | 100-400 |
| 217C | HTC3-2 | Solar energy | 150-350 |
| 217D | HTC3-2 | Solar energy | 200-300 |
| 218A | HTC3-2 | Geothermal water | NR |
| 218B | HTC3-2 | Geothermal water | 50-350 |
| 218C | HTC3-2 | Geothermal water | 100-300 |
| 218D | HTC3-2 | Geothermal water | 125-275 |
| 219A | HTC3-2 | Low pressure steam | NR |
| 219B | HTC3-2 | Low pressure steam | =<121 |
| 219C | HTC3-2 | Low pressure steam | =<115 |
| 219D | HTC3-2 | Low pressure steam | =<110 |
| 220A | HTC3-2 | distributed power generation equipment using fuel cells | NR |
| 220B | HTC3-2 | distributed power generation equipment using fuel cells | 60-200 |
| 220C | HTC3-2 | distributed power generation equipment using fuel cells | 80-200 |
| 220D | HTC3-2 | distributed power generation equipment using fuel cells | 100-200 |
| 221A | HTC3-2 | Exhaust gas from internaI combustion engine | NR |
| 221B | HTC3-2 | Exhaust gas from internaI combustion engine | =<500 |
| 221C | HTC3-2 | Exhaust gas from internaI combustion engine | =<450 |
| 221D | HTC3-2 | Exhaust gas from internaI combustion engine | =<400 |
| 222A | HTC3-2 | Industrial waste heat | NR |
| 222B | HTC3-3 | Industrial waste heat | 90-800 |
| 222C | HTC3-3 | Industrial waste heat | 90-400 |
| 222D | HTC3-3 | Industrial waste heat | 90-200 |
| 223A | HTC3-3 | Solar energy | NR |
| 223B | HTC3-3 | Solar energy | 100-400 |
| 223C | HTC3-3 | Solar energy | 150-350 |
| 223D | HTC3-3 | Solar energy | 200-300 |
| 224A | HTC3-3 | Geothermal water | NR |
| 224B | HTC3-3 | Geothermal water | 50-350 |
| 224C | HTC3-3 | Geothermal water | 100-300 |
| 224D | HTC3-3 | Geothermal water | 125-275 |
| 225A | HTC3-3 | Low Pressure steam | NR |
| 225B | HTC3-3 | Low pressure steam | =<121 |
| 225C | HTC3-3 | Low pressure steam | =<115 |
| 225D | HTC3-3 | Low pressure steam | =<110 |
| 226A | HTC3-3 | distributed power generation equipment using fuel cells | NR |
| 226B | HTC3-3 | distributed power generation equipment using fuel cells | 60-200 |
| 226C | HTC3-3 | distributed power generation equipment using fuel cells | 80-200 |
| 226D | HTC3-3 | distributed power generation equipment using fuel cells | 100-200 |
| 227A | HTC3-3 | Exhaust gas from internaI combustion engine | NR |
| 227B | HTC3-3 | Exhaust gas from internaI combustion engine | =<500 |
| 227C | HTC3-3 | Exhaust gas from internaI combustion engine | =<450 |
| 227D | HTC3-3 | Exhaust gas from internaI combustion engine | =<400 |
| 228A | HTC3-3 | Industrial waste heat | NR |
| 228B | HTC3-4 | Industrial waste heat | 90-800 |
| 228C | HTC3-4 | Industrial waste heat | 90-400 |
| 228D | HTC3-4 | Industrial waste heat | 90-200 |
| 229A | HTC3-4 | Solar energy | NR |
| 229B | HTC3-4 | Solar energy | 100-400 |
| 229C | HTC3-4 | Solar energy | 150-350 |
| 229D | HTC3-4 | Solar energy | 200-300 |
| 230A | HTC3-4 | Geothermal water | NR |
| 230B | HTC3-4 | Geothermal water | 50-350 |
| 230C | HTC3-4 | Geothermal water | 100-300 |
| 230D | HTC3-4 | Geothermal water | 125-275 |
| 231A | HTC3-4 | Low pressure steam | =<121 |
| 231B | HTC3-4 | Low pressure steam | =<115 |
| 231C | HTC3-4 | Low pressure steam | =<110 |
| 231D | HTC3-4 | Low pressure steam | NR |
| 232A | HTC3-4 | distributed power generation equipment using fuel cells | 60-200 |
| 232B | HTC3-4 | distributed power generation equipment using fuel cells | 80-200 |
| 232C | HTC3-4 | distributed power generation equipment using fuel cells | 100-200 |
| 232D | HTC3-4 | distributed power generation equipment using fuel cells | NR |
| 233A | HTC3-4 | Exhaust gas from internaI combustion engine | =<500 |
| 233B | HTC3-4 | Exhaust gas from internaI combustion engine | =<450 |
| 233C | HTC3-4 | Exhaust gas from internaI combustion engine | =<400 |
| 233D | HTC3-4 | Exhaust gas from internaI combustion engine | NR |
| 234A | HTC3-5 | Industrial waste heat | NR |
| 234B | HTC3-5 | Industrial waste heat | 90-800 |
| 234C | HTC3-5 | Industrial waste heat | 90-400 |
| 234D | HTC3-5 | Industrial waste heat | 90-200 |
| 235A | HTC3-6 | Solar energy | NR |
| 235B | HTC3-6 | Solar energy | 100-400 |
| 235C | HTC3-6 | Solar energy | 150-350 |
| 235D | HTC3-6 | Solar energy | 200-300 |
| 236A | HTC3-7 | Geothermal water | NR |
| 236B | HTC3-7 | Geothermal water | 50-350 |
| 236C | HTC3-7 | Geothermal water | 100-300 |
| 236D | HTC3-7 | Geothermal water | 125-275 |
| 237A | HTC4-1 | Low pressure steam | NR |
| 238B | HTC4-1 | Low pressure steam | =<121 |
| 238C | HTC4-1 | Low pressure steam | =<115 |
| 239D | HTC4-1 | Low pressure steam | =<110 |
| 240A | HTC4-1 | distributed power generation equipment using fuel cells | NR |
| 240B | HTC4-1 | distributed power generation equipment using fuel cells | 60-200 |
| 240C | HTC4-1 | distributed power generation equipment using fuel cells | 80-200 |
| 240D | HTC4-1 | distributed power generation equipment using fuel cells | 100-200 |
| 241A | HTC4-1 | Exhaust gas from internaI combustion engine | NR |
| 241B | HTC4-1 | Exhaust gas from internaI combustion engine | =<500 |
| 241C | HTC4-1 | Exhaust gas from internaI combustion engine | =<450 |
| 241D | HTC4-1 | Exhaust gas from internaI combustion engine | =<400 |
| 242A | HTC4-2 | Industrial waste heat | NR |
| 242B | HTC4-2 | Industrial waste heat | 90-800 |
| 242C | HTC4-2 | Industrial waste heat | 90-400 |
| 242D | HTC4-2 | Industrial waste heat | 90-200 |
| 243A | HTC4-2 | Solar energy | NR |
| 243B | HTC4-2 | Solar energy | 100-400 |
| 243C | HTC4-2 | Solar energy | 150-350 |
| 243D | HTC4-2 | Solar energy | 200-300 |
| 244A | HTC4-2 | Geothermal water | NR |
| 244B | HTC4-2 | Geothermal water | 50-350 |
| 244C | HTC4-2 | Geothermal water | 100-300 |
| 244D | HTC4-2 | Geothermal water | 125-275 |
| 244A | HTC4-2 | Low pressure steam | NR |
| 244B | HTC4-2 | Low pressure steam | =<121 |
| 245C | HTC4-2 | Low pressure steam | =<115 |
| 245D | HTC4-2 | Low pressure steam | =<110 |
| 246A | HTC4-2 | distributed power generation equipment using fuel cells | NR |
| 246B | HTC4-2 | distributed power generation equipment using fuel cells | 60-200 |
| 246C | HTC4-2 | distributed power generation equipment using fuel cells | 80-200 |
| 246D | HTC4-2 | distributed power generation equipment using fuel cells | 100-200 |
| 247A | HTC4-2 | Exhaust gas from internaI combustion engine | NR |
| 247B | HTC4-2 | Exhaust gas from internaI combustion engine | =<500 |
| 247C | HTC4-2 | Exhaust gas from internaI combustion engine | =<450 |
| 247D | HTC4-2 | Exhaust gas from internaI combustion engine | =<400 |
| 248A | HTC4-3 | Industrial waste heat | NR |
| 248B | HTC4-3 | Industrial waste heat | 90-800 |
| 248C | HTC4-3 | Industrial waste heat | 90-400 |
| 248D | HTC4-3 | Industrial waste heat | 90-200 |
| 249A | HTC4-3 | Solar energy | NR |
| 249B | HTC4-3 | Solar energy | 100-400 |
| 249C | HTC4-3 | Solar energy | 150-350 |
| 249D | HTC4-3 | Solar energy | 200-300 |
| 250A | HTC4-3 | Geothermal water | NR |
| 250B | HTC4-3 | Geothermal water | 50-350 |
| 250C | HTC4-3 | Geothermal water | 100-300 |
| 250D | HTC4-3 | Geothermal water | 125-275 |
| 251A | HTC4-3 | Low pressure steam | NR |
| 251B | HTC4-3 | Low pressure steam | =<121 |
| 251C | HTC4-3 | Low pressure steam | =<115 |
| 251D | HTC4-3 | Low pressure steam | =<110 |
| 252A | HTC4-3 | distributed power generation equipment using fuel cells | NR |
| 252B | HTC4-3 | distributed power generation equipment using fuel cells | 60-200 |
| 252C | HTC4-3 | distributed power generation equipment using fuel cells | 80-200 |
| 252D | HTC4-3 | distributed power generation equipment using fuel cells | 100-200 |
| 253A | HTC4-3 | Exhaust gas from internaI combustion engine | NR |
| 253B | HTC4-3 | Exhaust gas from internaI combustion engine | =<500 |
| 253C | HTC4-3 | Exhaust gas from internaI combustion engine | =<450 |
| 253D | HTC4-3 | Exhaust gas from internaI combustion engine | =<400 |
| 254A | HTC4-4 | Industrial waste heat | NR |
| 254B | HTC4-4 | Industrial waste heat | 90-800 |
| 254C | HTC4-4 | Industrial waste heat | 90-400 |
| 254D | HTC4-4 | Industrial waste heat | 90-200 |
| 256A | HTC4-4 | Solar energy | NR |
| 256B | HTC4-4 | Solar energy | 100-400 |
| 256C | HTC4-4 | Solar energy | 150-350 |
| 256D | HTC4-4 | Solar energy | 200-300 |
| 257A | HTC4-4 | Geothermal water | NR |
| 257B | HTC4-4 | Geothermal water | 50-350 |
| 257C | HTC4-4 | Geothermal water | 100-300 |
| 257D | HTC4-4 | Geothermal water | 125-275 |
| 258A | HTC4-4 | Low pressure steam | NR |
| 258B | HTC4-4 | Low pressure steam | =<121 |
| 258C | HTC4-4 | Low pressure steam | =<115 |
| 258D | HTC4-4 | Low pressure steam | =<110 |
| 259A | HTC4-4 | distributed power generation equipment using fuel cells | NR |
| 259B | HTC4-4 | distributed power generation equipment using fuel cells | 60-200 |
| 259C | HTC4-4 | distributed power generation equipment using fuel cells | 80-200 |
| 259D | HTC4-4 | distributed power generation equipment using fuel cells | 100-200 |
| 260A | HTC4-4 | Exhaust gas from internaI combustion engine | NR |
| 260B | HTC4-4 | Exhaust gas from internaI combustion engine | =<500 |
| 260C | HTC4-4 | Exhaust gas from internaI combustion engine | =<450 |
| 260D | HTC4-4 | Exhaust gas from internaI combustion engine | =<400 |
| 261A | HTC4-5 | Industrial waste heat | NR |
| 261B | HTC4-5 | Industrial waste heat | 90-800 |
| 261C | HTC4-5 | Industrial waste heat | 90-400 |
| 261D | HTC4-5 | Industrial waste heat | 90-200 |
| 262A | HTC4-5 | Solar energy | NR |
| 262B | HTC4-5 | Solar energy | 100-400 |
| 262C | HTC4-5 | Solar energy | 150-350 |
| 262D | HTC4-5 | Solar energy | 200-300 |
| 263A | HTC4-5 | Geothermal water | NR |
| 263B | HTC4-5 | Geothermal water | 50-350 |
| 263C | HTC4-5 | Geothermal water | 100-300 |
| 263D | HTC4-5 | Geothermal water | 125-275 |
| 264A | HTC4-5 | Low pressure steam | NR |
| 264B | HTC4-5 | Low pressure steam | =<121 |
| 264C | HTC4-5 | Low pressure steam | =<115 |
| 264D | HTC4-5 | Low pressure steam | =<110 |
| 265A | HTC4-5 | distributed power generation equipment using fuel cells | NR |
| 265B | HTC4-5 | distributed power generation equipment using fuel cells | 60-200 |
| 265C | HTC4-5 | distributed power generation equipment using fuel cells | 80-200 |
| 265D | HTC4-5 | distributed power generation equipment using fuel cells | 100-200 |
| 266A | HTC4-5 | Exhaust gas from internaI combustion engine | NR |
| 266B | HTC4-5 | Exhaust gas from internaI combustion engine | =<500 |
| 266C | HTC4-5 | Exhaust gas from internaI combustion engine | =<450 |
| 266D | HTC4-5 | Exhaust gas from internaI combustion engine | =<400 |
| 267A | HTC4-6 | Industrial waste heat | NR |
| 267B | HTC4-6 | Industrial waste heat | 90-800 |
| 267C | HTC4-6 | Industrial waste heat | 90-400 |
| 267D | HTC4-6 | Industrial waste heat | 90-200 |
| 268A | HTC4-6 | Solar energy | NR |
| 268B | HTC4-6 | Solar energy | 100-400 |
| 268C | HTC4-6 | Solar energy | 150-350 |
| 268D | HTC4-6 | Solar energy | 200-300 |
| 269A | HTC4-6 | Geothermal water | NR |
| 269B | HTC4-6 | Geothermal water | 50-350 |
| 269C | HTC4-6 | Geothermal water | 100-300 |
| 269D | HTC4-6 | Geothermal water | 125-275 |
| 270A | HTC4-6 | Low pressure steam | NR |
| 270B | HTC4-6 | Low pressure steam | =<121 |
| 270C | HTC4-6 | Low pressure steam | =<115 |
| 270D | HTC4-6 | Low pressure steam | =<110 |
| 271A | HTC4-6 | distributed power generation equipment using fuel cells | NR |
| 271B | HTC4-6 | distributed power generation equipment using fuel cells | 60-200 |
| 271C | HTC4-6 | distributed power generation equipment using fuel cells | 80-200 |
| 271D | HTC4-6 | distributed power generation equipment using fuel cells | 100-200 |
| 272A | HTC4-6 | Exhaust gas from internaI combustion engine | NR |
| 272B | HTC4-6 | Exhaust gas from internaI combustion engine | =<500 |
| 272C | HTC4-6 | Exhaust gas from internaI combustion engine | =<450 |
| 272D | HTC4-6 | Exhaust gas from internaI combustion engine | =<400 |
| 273A | HTC4-6 | Industrial waste heat | NR |
| 273B | HTC4-6 | Industrial waste heat | 90-800 |
| 273C | HTC4-6 | Industrial waste heat | 90-400 |
| 273D | HTC4-6 | Industrial waste heat | 90-200 |
| 274A | HTC4-6 | Solar energy | NR |
| 274B | HTC4-6 | Solar energy | 100-400 |
| 274C | HTC4-6 | Solar energy | 150-350 |
| 274D | HTC4-6 | Solar energy | 200-300 |
| 275A | HTC4-6 | Geothermal water | NR |
| 275B | HTC4-6 | Geothermal water | 50-350 |
| 275C | HTC4-6 | Geothermal water | 100-300 |
| 275D | HTC4-6 | Geothermal water | 125-275 |
| 276A | HTC4-6 | Low pressure steam | NR |
| 276B | HTC4-6 | Low pressure steam | =<121 |
| 276C | HTC4-6 | Low pressure steam | =<115 |
| 276D | HTC4-6 | Low pressure steam | =<110 |
| 277A | HTC4-6 | distributed power generation equipment using fuel cells | NR |
| 277B | HTC4-6 | distributed power generation equipment using fuel cells | 60-200 |
| 277C | HTC4-6 | distributed power generation equipment using fuel cells | 80-200 |
| 277D | HTC4-6 | distributed power generation equipment using fuel cells | 100-200 |
| 278A | HTC4-6 | Exhaust gas from internaI combustion engine | NR |
| 278B | HTC4-6 | Exhaust gas from internaI combustion engine | =<500 |
| 278C | HTC4-6 | Exhaust gas from internaI combustion engine | =<450 |
| 278D | HTC4-6 | Exhaust gas from internaI combustion engine | =<400 |
| 279A | HTC4-7 | Industrial waste heat | NR |
| 279B | HTC4-7 | Industrial waste heat | 90-800 |
| 279C | HTC4-7 | Industrial waste heat | 90-400 |
| 279D | HTC4-7 | Industrial waste heat | 90-200 |
| 280A | HTC4-7 | Solar energy | NR |
| 280B | HTC4-7 | Solar energy | 100-400 |
| 280C | HTC4-7 | Solar energy | 150-350 |
| 280D | HTC4-7 | Solar energy | 200-300 |
| 281A | HTC4-7 | Geothermal water | NR |
| 281B | HTC4-7 | Geothermal water | 50-350 |
| 281C | HTC4-7 | Geothermal water | 100-300 |
| 281D | HTC4-7 | Geothermal water | 125-275 |
| 282A | HTC4-7 | Low pressure steam | NR |
| 282B | HTC4-7 | Low pressure steam | =<121 |
| 282C | HTC4-7 | Low pressure steam | =<115 |
| 283D | HTC4-7 | Low pressure steam | =<110 |
| 284A | HTC4-7 | distributed power generation equipment using fuel cells | NR |
| 284B | HTC4-7 | distributed power generation equipment using fuel cells | 60-200 |
| 284C | HTC4-7 | distributed power generation equipment using fuel cells | 80-200 |
| 284D | HTC4-7 | distributed power generation equipment using fuel cells | 100-200 |
| 285A | HTC4-7 | Exhaust gas from internaI combustion engine | NR |
| 285B | HTC4-7 | Exhaust gas from internaI combustion engine | =<500 |
| 285C | HTC4-7 | Exhaust gas from internaI combustion engine | =<450 |
| 285D | HTC4-7 | Exhaust gas from internaI combustion engine | =<400 |
| 286A | HTC5-B1 | Industrial waste heat | NR |
| 286B | HTC5-B1 | Industrial waste heat | 90-800 |
| 286C | HTC5-B1 | Industrial waste heat | 90-400 |
| 286D | HTC5-B1 | Industrial waste heat | 90-200 |
| 287A | HTC5-B1 | Solar energy | NR |
| 287B | HTC5-B1 | Solar energy | 100-400 |
| 287C | HTC5-B1 | Solar energy | 150-350 |
| 287D | HTC5-B1 | Solar energy | 200-300 |
| 288A | HTC5-B1 | Geothermal water | NR |
| 288B | HTC5-B1 | Geothermal water | 50-350 |
| 288C | HTC5-B1 | Geothermal water | 100-300 |
| 288D | HTC5-B1 | Geothermal water | 125-275 |
| 289A | HTC5-B1 | Low pressure steam | NR |
| 289B | HTC5-B1 | Low pressure steam | =<121 |
| 289C | HTC5-B1 | Low pressure steam | =<115 |
| 289D | HTC5-B1 | Low pressure steam | =<110 |
| 290A | HTC5-B1 | distributed power generation equipment using fuel cells | NR |
| 290B | HTC5-B1 | distributed power generation equipment using fuel cells | 60-200 |
| 290C | HTC5-B1 | distributed power generation equipment using fuel cells | 80-200 |
| 290D | HTC5-B1 | distributed power generation equipment using fuel cells | 100-200 |
| 291A | HTC5-B1 | Exhaust gas from internaI combustion engine | NR |
| 291B | HTC5-B1 | Exhaust gas from internaI combustion engine | =<500 |
| 291C | HTC5-B1 | Exhaust gas from internaI combustion engine | =<450 |
| 291D | HTC5-B1 | Exhaust gas from internaI combustion engine | =<400 |
| 292A | HTC5-B2 | Industrial waste heat | NR |
| 292B | HTC5-B2 | Industrial waste heat | 90-800 |
| 292C | HTC5-B2 | Industrial waste heat | 90-400 |
| 292D | HTC5-B2 | Industrial waste heat | 90-200 |
| 293A | HTC5-B2 | Solar energy | NR |
| 293B | HTC5-B2 | Solar energy | 100-400 |
| 293C | HTC5-B2 | Solar energy | 150-350 |
| 293D | HTC5-B2 | Solar energy | 200-300 |
| 294A | HTC5-B2 | Geothermal water | NR |
| 294B | HTC5-B2 | Geothermal water | 50-350 |
| 294C | HTC5-B2 | Geothermal water | 100-300 |
| 294D | HTC5-B2 | Geothermal water | 125-275 |
| 294A | HTC5-B2 | Low pressure steam | NR |
| 295B | HTC5-B2 | Low pressure steam | =<121 |
| 295C | HTC5-B2 | Low pressure steam | =<115 |
| 295D | HTC5-B2 | Low pressure steam | =<110 |
| 295A | HTC5-B2 | distributed power generation equipment using fuel cells | NR |
| 296B | HTC5-B2 | distributed power generation equipment using fuel cells | 60-200 |
| 296C | HTC5-B2 | distributed power generation equipment using fuel cells | 80-200 |
| 296D | HTC5-B2 | distributed power generation equipment using fuel cells | 100-200 |
| 297A | HTC5-B2 | Exhaust gas from internaI combustion engine | NR |
| 297B | HTC5-B2 | Exhaust gas from internaI combustion engine | =<500 |
| 297C | HTC5-B2 | Exhaust gas from internaI combustion engine | =<450 |
| 297D | HTC5-B2 | Exhaust gas from internaI combustion engine | =<400 |
| 298A | HTC5-B2 | Industrial waste heat | NR |
| 298B | HTC5-B2 | Industrial waste heat | 90-800 |
| 298C | HTC5-B2 | Industrial waste heat | 90-400 |
| 298D | HTC5-B2 | Industrial waste heat | 90-200 |
| 299A | HTC5-B2 | Solar energy | NR |
| 299B | HTC5-B2 | Solar energy | 100-400 |
| 299C | HTC5-B2 | Solar energy | 150-350 |
| 299D | HTC5-B2 | Solar energy | 200-300 |
| 300A | HTC5-B2 | Geothermal water | NR |
| 300B | HTC5-B2 | Geothermal water | 50-350 |
| 300C | HTC5-B2 | Geothermal water | 100-300 |
| 300D | HTC5-B2 | Geothermal water | 125-275 |
| 301A | HTC5-B2 | Low pressure steam | NR |
| 301B | HTC5-B2 | Low pressure steam | =<121 |
| 301C | HTC5-B2 | Low pressure steam | =<115 |
| 301D | HTC5-B2 | Low pressure steam | =<110 |
| 302A | HTC5-B2 | distributed power generation equipment using fuel cells | NR |
| 302B | HTC5-B2 | distributed power generation equipment using fuel cells | 60-200 |
| 302C | HTC5-B2 | distributed power generation equipment using fuel cells | 80-200 |
| 302D | HTC5-B2 | distributed power generation equipment using fuel cells | 100-200 |
| 303A | HTC5-B2 | Exhaust gas from internaI combustion engine | NR |
| 303B | HTC5-B2 | Exhaust gas from internaI combustion engine | =<500 |
| 303C | HTC5-B2 | Exhaust gas from internaI combustion engine | =<450 |
| 303D | HTC5-B2 | Exhaust gas from internaI combustion engine | =<400 |
| 304A | HTC5-B3 | Industrial waste heat | NR |
| 304B | HTC5-B3 | Industrial waste heat | 90-800 |
| 304C | HTC5-B3 | Industrial waste heat | 90-400 |
| 304D | HTC5-B3 | Industrial waste heat | 90-200 |
| 305A | HTC5-B3 | Solar energy | NR |
| 305B | HTC5-B3 | Solar energy | 100-400 |
| 305C | HTC5-B3 | Solar energy | 150-350 |
| 305D | HTC5-B3 | Solar energy | 200-300 |
| 306A | HTC5-B3 | Geothermal water | NR |
| 306B | HTC5-B3 | Geothermal water | 50-350 |
| 306C | HTC5-B3 | Geothermal water | 100-300 |
| 306D | HTC5-B3 | Geothermal water | 125-275 |
| 307A | HTC5-B3 | Low pressure steam | NR |
| 307B | HTC5-B3 | Low pressure steam | =<121 |
| 307C | HTC5-B3 | Low pressure steam | =<115 |
| 307D | HTC5-B3 | Low pressure steam | =<110 |
| 308A | HTC5-B3 | distributed power generation equipment using fuel cells | NR |
| 308B | HTC5-B3 | distributed power generation equipment using fuel cells | 60-200 |
| 308C | HTC5-B3 | distributed power generation equipment using fuel cells | 80-200 |
| 308D | HTC5-B3 | distributed power generation equipment using fuel cells | 100-200 |
| 309A | HTC5-B3 | Exhaust gas from internaI combustion engine | NR |
| 309B | HTC5-B3 | Exhaust gas from internaI combustion engine | =<500 |
| 309C | HTC5-B3 | Exhaust gas from internaI combustion engine | =<450 |
| 309D | HTC5-B3 | Exhaust gas from internaI combustion engine | =<400 |
| 310A | HTC5-B4 | Industrial waste heat | NR |
| 310B | HTC5-B4 | Industrial waste heat | 90-800 |
| 310C | HTC5-B4 | Industrial waste heat | 90-400 |
| 310D | HTC5-B4 | Industrial waste heat | 90-200 |
| 311A | HTC5-B4 | Solar energy | NR |
| 311B | HTC5-B4 | Solar energy | 100-400 |
| 311C | HTC5-B4 | Solar energy | 150-350 |
| 311D | HTC5-B4 | Solar energy | 200-300 |
| 313A | HTC5-B4 | Geothermal water | NR |
| 312B | HTC5-B4 | Geothermal water | 50-350 |
| 312C | HTC5-B4 | Geothermal water | 100-300 |
| 312D | HTC5-B4 | Geothermal water | 125-275 |
| 313A | HTC5-B4 | Low pressure steam | NR |
| 313B | HTC5-B4 | Low pressure steam | =<121 |
| 313C | HTC5-B4 | Low pressure steam | =<115 |
| 313D | HTC5-B4 | Low pressure steam | =<110 |
| 314A | HTC5-B4 | distributed power generation equipment using fuel cells | NR |
| 314B | HTC5-B4 | distributed power generation equipment using fuel cells | 60-200 |
| 314C | HTC5-B4 | distributed power generation equipment using fuel cells | 80-200 |
| 314D | HTC5-B4 | distributed power generation equipment using fuel cells | 100-200 |
| 315A | HTC5-B4 | Exhaust gas from internaI combustion engine | NR |
| 315B | HTC5-B4 | Exhaust gas from internaI combustion engine | =<500 |
| 315C | HTC5-B4 | Exhaust gas from internaI combustion engine | =<450 |
| 315D | HTC5-B4 | Exhaust gas from internaI combustion engine | =<400 |
| 316A | HTC5-B5 | Industrial waste heat | NR |
| 316B | HTC5-B5 | Industrial waste heat | 90-800 |
| 316C | HTC5-B5 | Industrial waste heat | 90-400 |
| 316D | HTC5-B5 | Industrial waste heat | 90-200 |
| 317A | HTC5-B5 | Solar energy | NR |
| 317B | HTC5-B5 | Solar energy | 100-400 |
| 317C | HTC5-B5 | Solar energy | 150-350 |
| 317D | HTC5-B5 | Solar energy | 200-300 |
| 318A | HTC5-B5 | Geothermal water | NR |
| 318B | HTC5-B5 | Geothermal water | 50-350 |
| 318C | HTC5-B5 | Geothermal water | 100-300 |
| 318D | HTC5-B5 | Geothermal water | 125-275 |
| 319A | HTC5-B5 | Low pressure steam | NR |
| 319B | HTC5-B5 | Low pressure steam | =<121 |
| 319C | HTC5-B5 | Low pressure steam | =<115 |
| 319D | HTC5-B5 | Low pressure steam | =<110 |
| 320A | HTC5-B5 | distributed power generation equipment using fuel cells | NR |
| 320B | HTC5-B5 | distributed power generation equipment using fuel cells | 60-200 |
| 320C | HTC5-B5 | distributed power generation equipment using fuel cells | 80-200 |
| 320D | HTC5-B5 | distributed power generation equipment using fuel cells | 100-200 |
| 321A | HTC5-B5 | Exhaust gas from internaI combustion engine | NR |
| 321B | HTC5-B5 | Exhaust gas from internaI combustion engine | =<500 |
| 321C | HTC5-B5 | Exhaust gas from internaI combustion engine | =<450 |
| 321D | HTC5-B5 | Exhaust gas from internaI combustion engine | =<400 |
| 322A | HTC5-B6 | Industrial waste heat | NR |
| 322B | HTC5-B6 | Industrial waste heat | 90-800 |
| 322C | HTC5-B6 | Industrial waste heat | 90-400 |
| 322D | HTC5-B6 | Industrial waste heat | 90-200 |
| 323A | HTC5-B6 | Solar energy | NR |
| 323B | HTC5-B6 | Solar energy | 100-400 |
| 323C | HTC5-B6 | Solar energy | 150-350 |
| 323D | HTC5-B6 | Solar energy | 200-300 |
| 324A | HTC5-B6 | Geothermal water | NR |
| 324B | HTC5-B6 | Geothermal water | 50-350 |
| 324C | HTC5-B6 | Geothermal water | 100-300 |
| 324D | HTC5-B6 | Geothermal water | 125-275 |
| 325A | HTC5-B6 | Low Pressure steam | NR |
| 325B | HTC5-B6 | Low pressure steam | =<121 |
| 325C | HTC5-B6 | Low pressure steam | =<115 |
| 325D | HTC5-B6 | Low pressure steam | =<110 |
| 326A | HTC5-B6 | distributed power generation equipment using fuel cells | NR |
| 326B | HTC5-B6 | distributed power generation equipment using fuel cells | 60-200 |
| 326C | HTC5-B6 | distributed power generation equipment using fuel cells | 80-200 |
| 326D | HTC5-B6 | distributed power generation equipment using fuel cells | 100-200 |
| 327A | HTC5-B6 | Exhaust gas from internaI combustion engine | NR |
| 327B | HTC5-B6 | Exhaust gas from internaI combustion engine | =<500 |
| 327C | HTC5-B6 | Exhaust gas from internaI combustion engine | =<450 |
| 327D | HTC5-B6 | Exhaust gas from internaI combustion engine | =<400 |
| 328A | HTC5-B7 | Industrial waste heat | NR |
| 328B | HTC5-B7 | Industrial waste heat | 90-800 |
| 328C | HTC5-B7 | Industrial waste heat | 90-400 |
| 328D | HTC5-B7 | Industrial waste heat | 90-200 |
| 329A | HTC5-B7 | Solar energy | NR |
| 329B | HTC5-B7 | Solar energy | 100-400 |
| 329C | HTC5-B7 | Solar energy | 150-350 |
| 329D | HTC5-B7 | Solar energy | 200-300 |
| 330A | HTC5-B7 | Geothermal water | NR |
| 330B | HTC5-B7 | Geothermal water | 50-350 |
| 330C | HTC5-B7 | Geothermal water | 100-300 |
| 330D | HTC5-B7 | Geothermal water | 125-275 |
| 331A | HTC5-B7 | Low pressure steam | NR |
| 331B | HTC5-B7 | Low pressure steam | =<121 |
| 331C | HTC5-B7 | Low pressure steam | =<115 |
| 331D | HTC5-B7 | Low pressure steam | =<110 |
| 332A | HTC5-B7 | distributed power generation equipment using fuel cells | NR |
| 332B | HTC5-B7 | distributed power generation equipment using fuel cells | 60-200 |
| 332C | HTC5-B7 | distributed power generation equipment using fuel cells | 80-200 |
| 332D | HTC5-B7 | distributed power generation equipment using fuel cells | 100-200 |
| 333A | HTC5-B7 | Exhaust gas from internaI combustion engine | NR |
| 333B | HTC5-B7 | Exhaust gas from internaI combustion engine | =<500 |
| 333C | HTC5-B7 | Exhaust gas from internaI combustion engine | =<450 |
| 333D | HTC5-B7 | Exhaust gas from internaI combustion engine | =<400 |
| 334A | HTC6-1 | Low pressure steam | NR |
| 334B | HTC6-1 | Low pressure steam | 90-800 |
| 334C | HTC6-1 | Low pressure steam | 90-400 |
| 334D | HTC6-1 | Low pressure steam | 90-200 |
| 335A | HTC6-1 | distributed power generation equipment using fuel cells | NR |
| 335B | HTC6-1 | distributed power generation equipment using fuel cells | 90-800 |
| 335C | HTC6-1 | distributed power generation equipment using fuel cells | 90-400 |
| 335D | HTC6-1 | distributed power generation equipment using fuel cells | 90-200 |
| 336A | HTC6-1 | Exhaust gas from internaI combustion engine | NR |
| 336B | HTC6-1 | Exhaust gas from internaI combustion engine | 100-400 |
| 336C | HTC6-1 | Exhaust gas from internaI combustion engine | 150-350 |
| 336D | HTC6-1 | Exhaust gas from internaI combustion engine | 200-300 |
| 337A | HTC6-2 | Industrial waste heat | NR |
| 338B | HTC6-2 | Industrial waste heat | 90-800 |
| 338C | HTC6-2 | Industrial waste heat | 90-400 |
| 339D | HTC6-2 | Industrial waste heat | 90-200 |
| 340A | HTC6-2 | Solar energy | NR |
| 340B | HTC6-2 | Solar energy | 100-400 |
| 340C | HTC6-2 | Solar energy | 150-350 |
| 340D | HTC6-2 | Solar energy | 200-300 |
| 341A | HTC6-2 | Geothermal water | NR |
| 341B | HTC6-2 | Geothermal water | 50-350 |
| 341C | HTC6-2 | Geothermal water | 100-300 |
| 341D | HTC6-2 | Geothermal water | 125-275 |
| 342A | HTC6-2 | Low pressure steam | NR |
| 342B | HTC6-2 | Low pressure steam | =<121 |
| 342C | HTC6-2 | Low pressure steam | =<115 |
| 342D | HTC6-2 | Low pressure steam | =<110 |
| 343A | HTC6-2 | distributed power generation equipment using fuel cells | NR |
| 343B | HTC6-2 | distributed power generation equipment using fuel cells | 60-200 |
| 343C | HTC6-2 | distributed power generation equipment using fuel cells | 80-200 |
| 343D | HTC6-2 | distributed power generation equipment using fuel cells | 100-200 |
| 344A | HTC6-2 | Exhaust gas from internaI combustion engine | NR |
| 344B | HTC6-2 | Exhaust gas from internaI combustion engine | =<500 |
| 344C | HTC6-2 | Exhaust gas from internaI combustion engine | =<450 |
| 344D | HTC6-2 | Exhaust gas from internaI combustion engine | =<400 |
| 344A | HTC6-3 | Industrial waste heat | NR |
| 344B | HTC6-3 | Industrial waste heat | 90-800 |
| 345C | HTC6-3 | Industrial waste heat | 90-400 |
| 345D | HTC6-3 | Industrial waste heat | 90-200 |
| 346A | HTC6-3 | Solar energy | NR |
| 346B | HTC6-3 | Solar energy | 100-400 |
| 346C | HTC6-3 | Solar energy | 150-350 |
| 346D | HTC6-3 | Solar energy | 200-300 |
| 347A | HTC6-3 | Geothermal water | NR |
| 347B | HTC6-3 | Geothermal water | 50-350 |
| 347C | HTC6-3 | Geothermal water | 100-300 |
| 347D | HTC6-3 | Geothermal water | 125-275 |
| 348A | HTC6-3 | Low pressure steam | NR |
| 348B | HTC6-3 | Low pressure steam | =<121 |
| 348C | HTC6-3 | Low pressure steam | =<115 |
| 348D | HTC6-3 | Low pressure steam | =<110 |
| 349A | HTC6-3 | distributed power generation equipment using fuel cells | NR |
| 349B | HTC6-3 | distributed power generation equipment using fuel cells | 60-200 |
| 349C | HTC6-3 | distributed power generation equipment using fuel cells | 80-200 |
| 349D | HTC6-3 | distributed power generation equipment using fuel cells | 100-200 |
| 350A | HTC6-3 | Exhaust gas from internaI combustion engine | NR |
| 350B | HTC6-3 | Exhaust gas from internaI combustion engine | =<500 |
| 350C | HTC6-3 | Exhaust gas from internaI combustion engine | =<450 |
| 350D | HTC6-3 | Exhaust gas from internaI combustion engine | =<400 |
| 351A | HTC6-4 | Industrial waste heat | NR |
| 351B | HTC6-4 | Industrial waste heat | 90-800 |
| 351C | HTC6-4 | Industrial waste heat | 90-400 |
| 351D | HTC6-4 | Industrial waste heat | 90-200 |
| 352A | HTC6-4 | Solar energy | NR |
| 352B | HTC6-4 | Solar energy | 100-400 |
| 352C | HTC6-4 | Solar energy | 150-350 |
| 352D | HTC6-4 | Solar energy | 200-300 |
| 353A | HTC6-4 | Geothermal water | NR |
| 353B | HTC6-4 | Geothermal water | 50-350 |
| 353C | HTC6-4 | Geothermal water | 100-300 |
| 353D | HTC6-4 | Geothermal water | 125-275 |
| 354A | HTC6-4 | Low pressure steam | NR |
| 354B | HTC6-4 | Low pressure steam | =<121 |
| 354C | HTC6-4 | Low pressure steam | =<115 |
| 354D | HTC6-4 | Low pressure steam | =<110 |
| 356A | HTC6-4 | distributed power generation equipment using fuel cells | NR |
| 356B | HTC6-4 | distributed power generation equipment using fuel cells | 60-200 |
| 356C | HTC6-4 | distributed power generation equipment using fuel cells | 80-200 |
| 356D | HTC6-4 | distributed power generation equipment using fuel cells | 100-200 |
| 357A | HTC6-4 | Exhaust gas from internaI combustion engine | NR |
| 357B | HTC6-4 | Exhaust gas from internaI combustion engine | =<500 |
| 357C | HTC6-4 | Exhaust gas from internaI combustion engine | =<450 |
| 357D | HTC6-4 | Exhaust gas from internaI combustion engine | =<400 |
| 358A | HTC6-5 | Industrial waste heat | NR |
| 358B | HTC6-5 | Industrial waste heat | 90-800 |
| 358C | HTC6-5 | Industrial waste heat | 90-400 |
| 358D | HTC6-5 | Industrial waste heat | 90-200 |
| 359A | HTC6-5 | Solar energy | NR |
| 359B | HTC6-5 | Solar energy | 100-400 |
| 359C | HTC6-5 | Solar energy | 150-350 |
| 359D | HTC6-5 | Solar energy | 200-300 |
| 360A | HTC6-5 | Geothermal water | NR |
| 360B | HTC6-5 | Geothermal water | 50-350 |
| 360C | HTC6-5 | Geothermal water | 100-300 |
| 360D | HTC6-5 | Geothermal water | 125-275 |
| 361A | HTC6-5 | Low pressure steam | NR |
| 361B | HTC6-5 | Low pressure steam | =<121 |
| 361C | HTC6-5 | Low pressure steam | =<115 |
| 361D | HTC6-5 | Low pressure steam | =<110 |
| 362A | HTC6-5 | distributed power generation equipment using fuel cells | NR |
| 362B | HTC6-5 | distributed power generation equipment using fuel cells | 60-200 |
| 362C | HTC6-5 | distributed power generation equipment using fuel cells | 80-200 |
| 362D | HTC6-5 | distributed power generation equipment using fuel cells | 100-200 |
| 363A | HTC6-5 | Exhaust gas from internaI combustion engine | NR |
| 363B | HTC6-5 | Exhaust gas from internaI combustion engine | =<500 |
| 363C | HTC6-5 | Exhaust gas from internaI combustion engine | =<450 |
| 363D | HTC6-5 | Exhaust gas from internaI combustion engine | =<400 |
| 364A | HTC6-6 | Industrial waste heat | NR |
| 364B | HTC6-6 | Industrial waste heat | 90-800 |
| 364C | HTC6-6 | Industrial waste heat | 90-400 |
| 364D | HTC6-6 | Industrial waste heat | 90-200 |
| 365A | HTC6-6 | Solar energy | NR |
| 365B | HTC6-6 | Solar energy | 100-400 |
| 365C | HTC6-6 | Solar energy | 150-350 |
| 365D | HTC6-6 | Solar energy | 200-300 |
| 366A | HTC6-6 | Geothermal water | NR |
| 366B | HTC6-6 | Geothermal water | 50-350 |
| 366C | HTC6-6 | Geothermal water | 100-300 |
| 366D | HTC6-6 | Geothermal water | 125-275 |
| 367A | HTC6-6 | Low pressure steam | NR |
| 367B | HTC6-6 | Low pressure steam | =<121 |
| 367C | HTC6-6 | Low pressure steam | =<115 |
| 367D | HTC6-6 | Low pressure steam | =<110 |
| 368A | HTC6-6 | distributed power generation equipment using fuel cells | NR |
| 368B | HTC6-6 | distributed power generation equipment using fuel cells | 60-200 |
| 368C | HTC6-6 | distributed power generation equipment using fuel cells | 80-200 |
| 368D | HTC6-6 | distributed power generation equipment using fuel cells | 100-200 |
| 369A | HTC6-6 | Exhaust gas from internaI combustion engine | NR |
| 369B | HTC6-6 | Exhaust gas from internaI combustion engine | =<500 |
| 369C | HTC6-6 | Exhaust gas from internaI combustion engine | =<450 |
| 3709D | HTC6-6 | Exhaust gas from internaI combustion engine | =<400 |
| 370A | HTC6-6 | Industrial waste heat | NR |
| 370B | HTC6-6 | Industrial waste heat | 90-800 |
| 370C | HTC6-6 | Industrial waste heat | 90-400 |
| 370D | HTC6-6 | Industrial waste heat | 90-200 |
| 371A | HTC6-6 | Solar energy | NR |
| 371B | HTC6-6 | Solar energy | 100-400 |
| 371C | HTC6-6 | Solar energy | 150-350 |
| 371D | HTC6-6 | Solar energy | 200-300 |
| 372A | HTC6-6 | Geothermal water | NR |
| 372B | HTC6-6 | Geothermal water | 50-350 |
| 372C | HTC6-6 | Geothermal water | 100-300 |
| 372D | HTC6-6 | Geothermal water | 125-275 |
| 373A | HTC6-6 | Low pressure steam | NR |
| 373B | HTC6-6 | Low pressure steam | =<121 |
| 373C | HTC6-6 | Low pressure steam | =<115 |
| 373D | HTC6-6 | Low pressure steam | =<110 |
| 374A | HTC6-6 | distributed power generation equipment using fuel cells | NR |
| 374B | HTC6-6 | distributed power generation equipment using fuel cells | 60-200 |
| 374C | HTC6-6 | distributed power generation equipment using fuel cells | 80-200 |
| 374D | HTC6-6 | distributed power generation equipment using fuel cells | 100-200 |
| 375A | HTC6-6 | Exhaust gas from internaI combustion engine | NR |
| 375B | HTC6-6 | Exhaust gas from internaI combustion engine | =<500 |
| 375C | HTC6-6 | Exhaust gas from internaI combustion engine | =<450 |
| 375D | HTC6-6 | Exhaust gas from internaI combustion engine | =<400 |
| 376A | HTC6-7 | Industrial waste heat | NR |
| 376B | HTC6-7 | Industrial waste heat | 90-800 |
| 376C | HTC6-7 | Industrial waste heat | 90-400 |
| 376D | HTC6-7 | Industrial waste heat | 90-200 |
| 377A | HTC6-7 | Solar energy | NR |
| 377B | HTC6-7 | Solar energy | 100-400 |
| 377C | HTC6-7 | Solar energy | 150-350 |
| 377D | HTC6-7 | Solar energy | 200-300 |
| 378A | HTC6-7 | Geothermal water | NR |
| 378B | HTC6-7 | Geothermal water | 50-350 |
| 378C | HTC6-7 | Geothermal water | 100-300 |
| 378D | HTC6-7 | Geothermal water | 125-275 |
| 379A | HTC6-7 | Low pressure steam | NR |
| 379B | HTC6-7 | Low pressure steam | =<121 |
| 379C | HTC6-7 | Low pressure steam | =<115 |
| 379D | HTC6-7 | Low pressure steam | =<110 |
| 380A | HTC6-7 | distributed power generation equipment using fuel cells | NR |
| 380B | HTC6-7 | distributed power generation equipment using fuel cells | 60-200 |
| 380C | HTC6-7 | distributed power generation equipment using fuel cells | 80-200 |
| 380D | HTC6-7 | distributed power generation equipment using fuel cells | 100-200 |
| 381A | HTC6-7 | Exhaust gas from internaI combustion engine | NR |
| 381B | HTC6-7 | Exhaust gas from internaI combustion engine | =<500 |
| 381C | HTC6-7 | Exhaust gas from internaI combustion engine | =<450 |
| 381D | HTC6-7 | Exhaust gas from internaI combustion engine | =<400 |

### High Temperature Heat Pump

As discussed above, when a heat transfer fluid of the present invention, including each of Compositions 1-5 and HTCs 1-6, used in a heat pump, it is referred to as a refrigerant. The refrigerant therefore corresponds to the heat transfer fluid as discussed in this application. All preferred features of the heat transfer fluid as described apply to the refrigerant as described herein.

The refrigerant or heat transfer composition of the invention, including each of Compositions 1-5 and HTCs 1-6, may be used in a high temperature heat pump system.

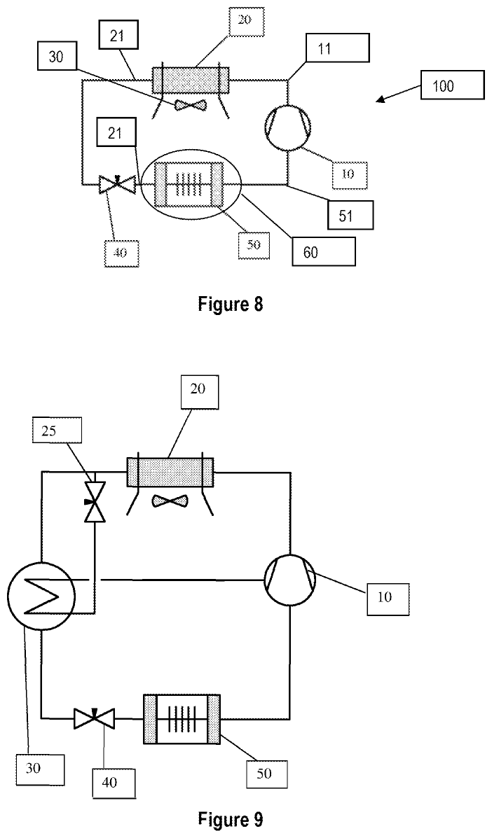

FIG. 8 illustrates in block diagram form a HTHP 100 according to the present invention includes an evaporator 50 that receives heat from the low temperature heat source (represented schematically by the oval 60), including by undergoing a phase change from liquid to vapor as heat is absorbed from the low temperature heat source. It will be appreciated, however, that some level of sensible heat may also transferred to the refrigerants of the present invention, including each of Refrigerants 1-5 and HTCs 1-6, by the low temperature heat source. The vaporous refrigerant which exits evaporator 50 via line 51 is introduced to the suction side of compressor 10 which adds work to the refrigerant and increases both the temperature and the pressure of the refrigerant vapor. This high temperature vapor from the compressor 10 is transported via line 11 to the condenser 20 in which the refrigerant of the present invention, including each of Refrigerants 1-5 and HTCs 1-6, supplies heat at a relatively high temperature to the high temperature heat sink, which is represented schematically (but not by way of limitation) by fan 30. The condensed refrigerant of the present invention then is transported by line 21 to a pressure reducing device, such as an expansion valve, 40 where the pressure of the liquid refrigerant is reduced, thus producing relatively low temperature liquid refrigerant, which is then introduced via line 41 to the condenser, where the cycle begins again.

An alternative but preferred embodiment of the present HTHP of the present invention is illustrated generally in FIG. 9. In this embodiment, the basic structure of the HTHP as described above is used, except a vapor injector economizer, represented schematically by the combination of the expansion valve 25 and heat exchanger 30, is used. In this arrangement, a portion of the liquid refrigerant of the present invention, including each of Compositions 1-5 and HTCs 1-6, exiting from the condenser 20 flows through the expansion valve 25 to produce a relative cool, low pressure liquid that feeds heat exchanger 30. In heat exchanger 30, the cool low pressure liquid absorbs heat from the relatively warm condenser liquid by vaporizing to form a relatively low pressure refrigerant vapor which then enters into a suction side of the compressor 10. The condenser liquid which has been cooled in the vapor injector economizer then proceeds to the expansion valve 40.

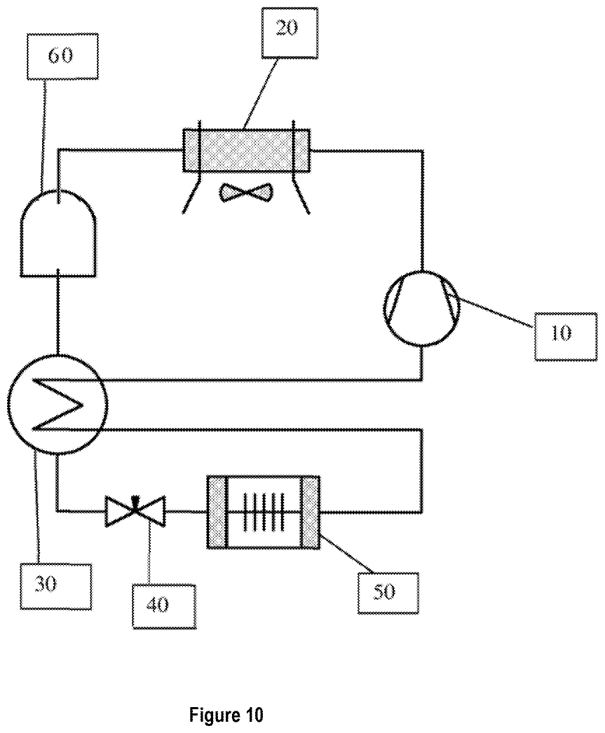

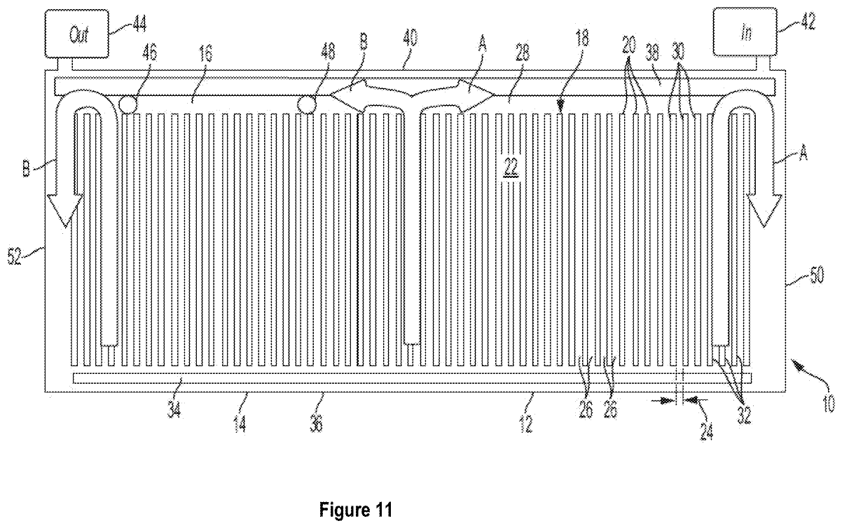

Another alternative but preferred embodiment of the present HTHP of the present invention is illustrated generally in FIG. 10. In this embodiment, the basic structure of the HTHP as described above in FIG. 1 is used, except a suction line heat exchanger (SLHX) 30 is used. In this arrangement, at least a portion of the liquid refrigerant of the present invention, including each of Compositions 1-5 and HTCs 1-6, exiting from the condenser 20 flow to an accumulator and/or oil separator 60 and then to SLHX 30 wherein heat is introduced into the feed line to the compressor 10. The heat transfer fluid, including heat transfer compositions including Refrigerants 1-5 and HTCs 1-6, exiting the SLHX 30 then is introduced to the expansion valve 25 to produce a relative cool, low pressure liquid that feeds heat exchanger 50. In heat exchanger 50, the cool low pressure liquid absorbs heat from the relatively warm condenser liquid by vaporizing to form a relatively low pressure refrigerant vapor which then, after passing through the SLHX 30, enters into a suction side of the compressor 10.

The specific type of equipment used in the present heat pump systems can vary widely within the scope of the present invention. For example, the compressor can be of centrifugal, screw and positive displacement type. In preferred aspects, the high temperature heat pump systems of the present invention, including each of Heat Transfer Methods 1, and HTHP Method Nos. 1-129 (as defined in the Table 5 which follows below), comprise one or more single stage centrifugal compressors and/or one or more multistage centrifugal compressors, and combinations of one or more single stage and one or more multistage centrifugal compressors. In certain aspects, particularly those centrifugal compressor(s) as described in the previous paragraph, a vapor injector economizer (VIE) is used. In certain aspects, particularly those centrifugal compressor(s) as described in the previous paragraph which comprise one or more multistage compressors, the VIE introduces vapor to the compressor between one or more of said multiple stages. In certain aspects, particularly those centrifugal compressor(s) as described in the previous paragraph which comprise one or more 2 stage compressors, a VIE is provided which introduces vapor to the compressor at least between the first and second stage. In certain aspects, particularly those centrifugal compressor(s) as described in the previous paragraph which comprise one or more 3 stage compressors, a VIE is provided which introduces vapor to the compressor at least between the first and second stage and/or at least between the second and third stage.

With respect to the heat exchangers 20, 30 and 50, applicants note that the preferred refrigerants of the present invention have condenser glides and evaporator glides of from about 3 °C to less than about 15 °C, and for the use of the refrigerants of the present invention, including each of Compositions 1-5 and HTCs 1-6, it is preferred that counter-current and/or cross current flow heat exchangers are used for evaporator(s) and the condenser(s). Applicants have come to appreciate that the use of such heat exchangers in the systems and methods as described herein allow advantageous use of glide matching with the heat sink or heat source. Furthermore, the use of heat exchangers that do not permit fractionation during evaporation or condensation are preferred, and accordingly flooded heat exchangers are preferably not used on the refrigerant side and instead heat exchangers such as brazed plate heat exchangers, and similar heat exchangers which do not permit fractionation of refrigerant, are preferred. For example, if shell and tube heat exchangers are used for either the condenser or the evaporator, it is highly preferred that the refrigerant flows through the tubes in the heat exchanger and not on the shell side. Thus, the heat exchangers 20, 30 and 50 of the present systems and methods are preferably a dry expansion or direct expansion type (and not a flooded type) and in the case of the evaporator 50 produce a vapor with a super heat of at least about 5 °C and in the case of the condenser 20 a liquid with subcooling at least about 5 °C.

The type of expansion device used can vary. The expansion device can be an expansion valve, which can be electronic or thermostatic as needed by the specifics of the design. This description does not limit any possible additional variances for the specific equipment or the use of additional components that are not illustrated in FIG. 8, such as suction line heat exchangers, vapor ejectors and the like.

The following table identifies preferred high temperature heat pump method of the present invention (identified by the HTHP Method numbers in column 1) using: (1) the refrigerants of the present invention (identified in column 2 by numbers corresponding to the Composition Number (abbreviated Comp. No. in the first row of column 2) as defined above); (2) a direct expansion evaporator; (3) a vapor injector economizer; and (4) having the operating parameters as specified in the table. In the column headings, the following abbreviations are used: “LTHS Temp.” means the low temperature heat source temperature range; and “HTHS. Temp.” means high temperature heat source temperature range.

TABLE 5
HIGH TEMPERATURE HEAT PUMP METHOD CONDITIONS

| | | | | High | |
|---|---|---|---|---|---|
| HTHP | | | LTHS. | Temp. | HTHS. |
| Method | Comp. | | Temp., | Heat | Temp., |
| No. | No. | Low Temperature Heat Source | °C | Sink | °C |
| 1A | 1 | Industrial waste heat | 30-90 | Industrial | 110-145 |
| | | | | Steam | |
| 1B | 1 | Industrial waste heat | 30-90 | Low | 115-130 |
| | | | | Pressure | |
| | | | | Steam | |
| 1C | 1 | Industrial waste heat | 30-90 | Hot | 50-70 |
| | | | | water | |
| 1D | 1 | Industrial waste heat | 30-90 | Hot air | 35-60 |
| 2A | 1 | Industrial waste heat | 30-90 | Industrial | 110-145 |
| | | from data centers | | Steam | |
| 2B | 1 | Industrial waste heat | 30-90 | Low | 115-130 |
| | | from data centers | | Pressure | |
| | | | | Steam | |
| 2C | 1 | Industrial waste heat | 30-90 | Hot | 50-70 |
| | | from data centers | | water | |
| 2D | 1 | Industrial waste heat from data centers | 30-90 | Hot air | 35-60 |
| 3A | 1 | Industrial waste heat from data centers | 30-90 | Industrial | 110-145 |
| | | | | Steam | |
| 3B | 1 | Industrial waste heat from data centers | 30-90 | Low | 115-130 |
| | | | | Pressure | |
| | | | | Steam | |
| 3C | 1 | Industrial waste heat from data centers | 30-90 | Hot | 50-70 |
| | | | | water | |
| 3D | 1 | Industrial waste heat from data centers | 30-90 | Hot air | 35-60 |
| 4A | 1 | Industrial waste heat from beverage | 30-90 | Industrial | 110-145 |
| | | manufacturing and/or bottling | | Steam | |
| 4B | 1 | Industrial waste heat from beverage | 30-90 | Low | 115-130 |
| | | manufacturing and/or bottling | | Pressure | |
| | | | | Steam | |
| 4C | 1 | Industrial waste heat from beverage | 30-90 | Hot | 50-70 |
| | | manufacturing and/or bottling | | water | |
| 4D | 1 | Industrial waste heat from beverage | 30-90 | Hot air | 35-60 |
| | | manufacturing and/or bottling | | | |
| 5A | 1 | Industrial waste heat from beverage | 30-90 | Industrial | 110-145 |
| | | manufacturing and/or bottling | | Steam | |
| 5B | 1 | Industrial waste heat from beverage | 30-90 | Low | 115-130 |
| | | manufacturing and/or bottling | | Pressure | |
| | | | | Steam | |
| 5C | 1 | Industrial waste heat from beverage | 30-90 | Hot | 50-70 |
| | | manufacturing and/or bottling | | water | |
| 5D | 1 | Industrial waste heat from beverage | 30-90 | Hot air | 35-60 |
| | | manufacturing and/or processing | | | |
| 6A | 1 | Industrial waste heat from dairy | 30-90 | Industrial | 110-145 |
| | | manufacturing and/or processing | | Steam | |
| 6B | 1 | Industrial waste heat from dairy | 30-90 | Low | 115-130 |
| | | manufacturing and/or processing | | Pressure | |
| | | | | Steam | |
| 6C | 1 | Industrial waste heat from dairy | 30-90 | Hot | 50-70 |
| | | manufacturing and/or processing | | water | |
| 6D | 1 | Industrial waste heat from dairy | 30-90 | Hot air | 35-60 |
| | | manufacturing and/or processing | | | |
| 7A | 2A | Industrial waste heat | 30-90 | Industrial | 110-145 |
| | | | | Steam | |
| 7B | 2A | Industrial waste heat | 30-90 | Low | 115-130 |
| | | | | Pressure | |
| | | | | Steam | |
| 7C | 2A | Industrial waste heat | 30-90 | Hot | 50-70 |
| | | | | water | |
| 7D | 2A | Industrial waste heat | 30-90 | Hot air | 35-60 |
| 8A | 2A | Industrial waste heat from data centers | 30-90 | Industrial | 110-145 |
| | | | | Steam | |
| 8B | 2A | Industrial waste heat from data centers | 30-90 | Low | 115-130 |
| | | | | Pressure | |
| | | | | Steam | |
| 8C | 2A | Industrial waste heat from data centers | 30-90 | Hot | 50-70 |
| | | | | water | |
| 8D | 2A | Industrial waste heat from data centers | 30-90 | Hot air | 35-60 |
| 9A | 2A | Industrial waste heat from data centers | 30-90 | Industrial | 110-145 |
| | | | | Steam | |
| 9B | 2A | Industrial waste heat from data centers | 30-90 | Low | 115-130 |
| | | | | Pressure | |
| | | | | Steam | |
| 9C | 2A | Industrial waste heat from data centers | 30-90 | Hot | 50-70 |
| | | | | water | |
| 9D | 2A | Industrial waste heat from data centers | 30-90 | Hot air | 35-60 |
| 10A | 2A | Industrial waste heat from beverage | 30-90 | Industrial | 110-145 |
| | | manufacturing and/or bottling | | Steam | |
| 10B | 2A | Industrial waste heat from beverage | 30-90 | Low | 115-130 |
| | | manufacturing and/or bottling | | Pressure | |
| | | | | Steam | |
| 10C | 2A | Industrial waste heat from beverage | 30-90 | Hot | 50-70 |
| | | manufacturing and/or bottling | | water | |
| 10D | 2A | Industrial waste heat from beverage | 30-90 | Hot air | 35-60 |
| | | manufacturing and/or bottling | | | |
| 11A | 2A | Industrial waste heat from beverage | 30-90 | Industrial | 110-145 |
| | | manufacturing and/or bottling | | Steam | |
| 11B | 2A | Industrial waste heat from beverage | 30-90 | Low | 115-130 |
| | | manufacturing and/or bottling | | Pressure | |
| | | | | Steam | |
| 11C | 2A | Industrial waste heat from beverage | 30-90 | Hot | 50-70 |
| | | manufacturing and/or bottling | | water | |
| 11D | 2A | Industrial waste heat from beverage | 30-90 | Hot air | 35-60 |
| | | manufacturing and/or processing | | | |
| 12A | 3B | Industrial waste heat from dairy | 30-90 | Industrial | 110-145 |
| | | manufacturing and/or processing | | Steam | |
| 12B | 3B | Industrial waste heat from dairy | 30-90 | Low | 115-130 |
| | | manufacturing and/or processing | | Pressure | |
| | | | | Steam | |
| 12C | 3B | Industrial waste heat from dairy | 30-90 | Hot | 50-70 |
| | | manufacturing and/or processing | | water | |
| 12D | 3B | Industrial waste heat from dairy | 30-90 | Hot air | 35-60 |
| | | manufacturing and/or processing | | | |
| 13A | 3B | Industrial waste heat | 30-90 | Industrial | 110-145 |
| | | | | Steam | |
| 13B | 3B | Industrial waste heat | 30-90 | Low | 115-130 |
| | | | | Pressure | |
| | | | | Steam | |
| 13C | 3B | Industrial waste heat | 30-90 | Hot | 50-70 |
| | | | | water | |
| 13D | 3B | Industrial waste heat | 30-90 | Hot air | 35-60 |
| 14A | 3B | Industrial waste heat from data centers | 30-90 | Industrial | 110-145 |
| | | | | Steam | |
| 14B | 3B | Industrial waste heat from data centers | 30-90 | Low | 115-130 |
| | | | | Pressure | |
| | | | | Steam | |
| 140 | 3B | Industrial waste heat from data centers | 30-90 | Hot | 50-70 |
| | | | | water | |
| 14D | 3B | Industrial waste heat from data centers | 30-90 | Hot air | 35-60 |
| 15A | 3B | Industrial waste heat from data centers | 30-90 | Industrial | 110-145 |
| | | | | Steam | |
| 15B | 3B | Industrial waste heat from data centers | 30-90 | Low | 115-130 |
| | | | | Pressure | |
| | | | | Steam | |
| 15C | 3B | Industrial waste heat from data centers | 30-90 | Hot | 50-70 |
| | | | | water | |
| 15D | 3B | Industrial waste heat from data centers | 30-90 | Hot air | 35-60 |
| 16A | 3B | Industrial waste heat from beverage | 30-90 | Industrial | 110-145 |
| | | manufacturing and/or bottling | | Steam | |
| 16B | 3B | Industrial waste heat from beverage | 30-90 | Low | 115-130 |
| | | manufacturing and/or bottling | | Pressure | |
| | | | | Steam | |
| 16C | 3B | Industrial waste heat from beverage | 30-90 | Hot | 50-70 |
| | | manufacturing and/or bottling | | water | |
| 16D | 3B | Industrial waste heat from beverage | 30-90 | Hot air | 35-60 |
| | | manufacturing and/or bottling | | | |
| 17A | 3B | Industrial waste heat from beverage | 30-90 | Industrial | 110-145 |
| | | manufacturing and/or bottling | | Steam | |
| 17B | 3B | Industrial waste heat from beverage | 30-90 | Low | 115-130 |
| | | manufacturing and/or bottling | | Pressure | |
| | | | | Steam | |
| 17C | 3B | Industrial waste heat from beverage | 30-90 | Hot | 50-70 |
| | | manufacturing and/or bottling | | water | |
| 17D | 3B | Industrial waste heat from beverage | 30-90 | Hot air | 35-60 |
| | | manufacturing and/or processing | | | |
| 18A | 3B | Industrial waste heat from dairy | 30-90 | Industrial | 110-145 |
| | | manufacturing and/or processing | | Steam | |
| 18B | 3B | Industrial waste heat from dairy | 30-90 | Low | 115-130 |
| | | manufacturing and/or processing | | Pressure | |
| | | | | Steam | |
| 18C | 3B | Industrial waste heat from dairy | 30-90 | Hot | 50-70 |
| | | manufacturing and/or processing | | water | |
| 18D | 3B | Industrial waste heat from dairy | 30-90 | Hot air | 35-60 |
| | | manufacturing and/or processing | | | |
| 19A | 3Bt | Industrial waste heat | 30-90 | Industrial | 110-145 |
| | | | | Steam | |
| 19B | 3Bt | Industrial waste heat | 30-90 | Low | 115-130 |
| | | | | Pressure | |
| | | | | Steam | |
| 19C | 3Bt | Industrial waste heat | 30-90 | Hot | 50-70 |
| | | | | water | |
| 19D | 3Bt | Industrial waste heat | 30-90 | Hot air | 35-60 |
| 20A | 3Bt | Industrial waste heat from data centers | 30-90 | Industrial | 110-145 |
| | | | | Steam | |
| 20B | 3Bt | Industrial waste heat from data centers | 30-90 | Low | 115-130 |
| | | | | Pressure | |
| | | | | Steam | |
| 20C | 3Bt | Industrial waste heat from data centers | 30-90 | Hot | 50-70 |
| | | | | water | |
| 20D | 3Bt | Industrial waste heat from data centers | 30-90 | Hot air | 35-60 |
| 21A | 3Bt | Industrial waste heat from data centers | 30-90 | Industrial | 110-145 |
| | | | | Steam | |
| 21B | 3Bt | Industrial waste heat from data centers | 30-90 | Low | 115-130 |
| | | | | Pressure | |
| | | | | Steam | |
| 21C | 3Bt | Industrial waste heat from data centers | 30-90 | Hot | 50-70 |
| | | | | water | |
| 21D | 3Bt | Industrial waste heat from data centers | 30-90 | Hot air | 35-60 |
| 22A | 3Bt | Industrial waste heat from beverage | 30-90 | Industrial | 110-145 |
| | | manufacturing and/or bottling | | Steam | |
| 22B | 3Bt | Industrial waste heat from beverage | 30-90 | Low | 115-130 |
| | | manufacturing and/or bottling | | Pressure | |
| | | | | Steam | |
| 22C | 3Bt | Industrial waste heat from beverage | 30-90 | Hot | 50-70 |
| | | manufacturing and/or bottling | | water | |
| 22D | 3Bt | Industrial waste heat from beverage | 30-90 | Hot air | 35-60 |
| | | manufacturing and/or bottling | | | |
| 23A | 3Bt | Industrial waste heat from beverage | 30-90 | Industrial | 110-145 |
| | | manufacturing and/or bottling | | Steam | |
| 23B | 3Bt | Industrial waste heat from beverage | 30-90 | Low | 115-130 |
| | | manufacturing and/or bottling | | Pressure | |
| | | | | Steam | |
| 23C | 3Bt | Industrial waste heat from beverage | 30-90 | Hot | 50-70 |
| | | manufacturing and/or bottling | | water | |
| 23D | 3Bt | Industrial waste heat from beverage | 30-90 | Hot air | 35-60 |
| | | manufacturing and/or processing | | | |
| 24A | 3Bt | Industrial waste heat from dairy | 30-90 | Industrial | 110-145 |
| | | manufacturing and/or processing | | Steam | |
| 24B | 3Bt | Industrial waste heat from dairy | 30-90 | Low | 115-130 |
| | | manufacturing and/or processing | | Pressure | |
| | | | | Steam | |
| 24C | 3Bt | Industrial waste heat from dairy | 30-90 | Hot | 50-70 |
| | | manufacturing and/or processing | | water | |
| 24D | 3Bt | Industrial waste heat from dairy | 30-90 | Hot air | 35-60 |
| | | manufacturing and/or processing | | | |
| 25A | 3Bt | Industrial waste heat | 30-90 | Industrial | 110-145 |
| | | | | Steam | |
| 25B | 3Bt | Industrial waste heat | 30-90 | Low | 115-130 |
| | | | | Pressure | |
| | | | | Steam | |
| 25C | 3Bt | Industrial waste heat | 30-90 | Hot | 50-70 |
| | | | | water | |
| 25D | 3Bt | Industrial waste heat | 30-90 | Hot air | 35-60 |
| 26A | 3Bt | Industrial waste heat from data centers | 30-90 | Industrial | 110-145 |
| | | | | Steam | |
| 26B | 3Bt | Industrial waste heat from data centers | 30-90 | Low | 115-130 |
| | | | | Pressure | |
| | | | | Steam | |
| 26C | 3Bt | Industrial waste heat from data centers | 30-90 | Hot | 50-70 |
| | | | | water | |
| 26D | 3Bt | Industrial waste heat from data centers | 30-90 | Hot air | 35-60 |
| 27A | 3Bt | Industrial waste heat from data centers | 30-90 | Industrial | 110-145 |
| | | | | Steam | |
| 27B | 3Bt | Industrial waste heat from data centers | 30-90 | Low | 115-130 |
| | | | | Pressure | |
| | | | | Steam | |
| 27C | 3Bt | Industrial waste heat from data centers | 30-90 | Hot | 50-70 |
| | | | | water | |
| 27D | 3Bt | Industrial waste heat from data centers | 30-90 | Hot air | 35-60 |
| 28A | 3Bt | Industrial waste heat from beverage | 30-90 | Industrial | 110-145 |
| | | manufacturing and/or bottling | | Steam | |
| 28B | 3Bt | Industrial waste heat from beverage | 30-90 | Low | 115-130 |
| | | manufacturing and/or bottling | | Pressure | |
| | | | | Steam | |
| 28C | 3Bt | Industrial waste heat from beverage | 30-90 | Hot | 50-70 |
| | | manufacturing and/or bottling | | water | |
| 28D | 3Bt | Industrial waste heat from beverage | 30-90 | Hot air | 35-60 |
| | | manufacturing and/or bottling | | | |
| 29A | 3Bt | Industrial waste heat from beverage | 30-90 | Industrial | 110-145 |
| | | manufacturing and/or bottling | | Steam | |
| 29B | 3Bt | Industrial waste heat from beverage | 30-90 | Low | 115-130 |
| | | manufacturing and/or bottling | | Pressure | |
| | | | | Steam | |
| 29C | 3Bt | Industrial waste heat from beverage | 30-90 | Hot | 50-70 |
| | | manufacturing and/or bottling | | water | |
| 29D | 3Bt | Industrial waste heat from beverage | 30-90 | Hot air | 35-60 |
| | | manufacturing and/or processing | | | |
| 30A | 3Bt | Industrial waste heat from dairy | 30-90 | Industrial | 110-145 |
| | | manufacturing and/or processing | | Steam | |
| 30B | 3Bt | Industrial waste heat from dairy | 30-90 | Low | 115-130 |
| | | manufacturing and/or processing | | Pressure | |
| | | | | Steam | |
| 30C | 3Bt | Industrial waste heat from dairy | 30-90 | Hot | 50-70 |
| | | manufacturing and/or processing | | water | |
| 30D | 3Bt | Industrial waste heat from dairy | 30-90 | Hot air | 35-60 |
| | | manufacturing and/or processing | | | |
| 31A | 3Bc | Industrial waste heat | 30-90 | Industrial | 110-145 |
| | | | | Steam | |
| 31B | 3Bc | Industrial waste heat | 30-90 | Low | 115-130 |
| | | | | Pressure | |
| | | | | Steam | |
| 31C | 3Bc | Industrial waste heat | 30-90 | Hot | 50-70 |
| | | | | water | |
| 31D | 3Bc | Industrial waste heat | 30-90 | Hot air | 35-60 |
| 32A | 3Bc | Industrial waste heat from data centers | 30-90 | Industrial | 110-145 |
| | | | | Steam | |
| 32B | 3Bc | Industrial waste heat from data centers | 30-90 | Low | 115-130 |
| | | | | Pressure | |
| | | | | Steam | |
| 32C | 3Bc | Industrial waste heat from data centers | 30-90 | Hot | 50-70 |
| | | | | water | |
| 32D | 3Bc | Industrial waste heat from data centers | 30-90 | Hot air | 35-60 |
| 33A | 3Bc | Industrial waste heat from data centers | 30-90 | Industrial | 110-145 |
| | | | | Steam | |
| 33B | 3Bc | Industrial waste heat from data centers | 30-90 | Low | 115-130 |
| | | | | Pressure | |
| | | | | Steam | |
| 33C | 3Bc | Industrial waste heat from data centers | 30-90 | Hot | 50-70 |
| | | | | water | |
| 33D | 3Bc | Industrial waste heat from data centers | 30-90 | Hot air | 35-60 |
| 34A | 3Bc | Industrial waste heat from beverage | 30-90 | Industrial | 110-145 |
| | | manufacturing and/or bottling | | Steam | |
| 35B | 3Bc | Industrial waste heat from beverage | 30-90 | Low | 115-130 |
| | | manufacturing and/or bottling | | Pressure | |
| | | | | Steam | |
| 35C | 3Bc | Industrial waste heat from beverage | 30-90 | Hot | 50-70 |
| | | manufacturing and/or bottling | | water | |
| 35D | 3Bc | Industrial waste heat from beverage | 30-90 | Hot air | 35-60 |
| | | manufacturing and/or bottling | | | |
| 36A | 3Bc | Industrial waste heat from beverage | 30-90 | Industrial | 110-145 |
| | | manufacturing and/or bottling | | Steam | |
| 36B | 3Bc | Industrial waste heat from beverage | 30-90 | Low | 115-130 |
| | | manufacturing and/or bottling | | Pressure | |
| | | | | Steam | |
| 36C | 3Bc | Industrial waste heat from beverage | 30-90 | Hot | 50-70 |
| | | manufacturing and/or bottling | | water | |
| 36D | 3Bc | Industrial waste heat from beverage | 30-90 | Hot air | 35-60 |
| | | manufacturing and/or processing | | | |
| 37A | 3Bc | Industrial waste heat from dairy | 30-90 | Industrial | 110-145 |
| | | manufacturing and/or processing | | Steam | |
| 37B | 3Bc | Industrial waste heat from dairy | 30-90 | Low | 115-130 |
| | | manufacturing and/or processing | | Pressure | |
| | | | | Steam | |
| 37C | 3Bc | Industrial waste heat from dairy | 30-90 | Hot | 50-70 |
| | | manufacturing and/or processing | | water | |
| 37D | 3Bc | Industrial waste heat from dairy | 30-90 | Hot air | 35-60 |
| | | manufacturing and/or processing | | | |
| 38A | 3C | Industrial waste heat | 30-90 | Industrial | 110-145 |
| | | | | Steam | |
| 38B | 3C | Industrial waste heat | 30-90 | Low | 115-130 |
| | | | | Pressure | |
| | | | | Steam | |
| 38C | 3C | Industrial waste heat | 30-90 | Hot | 50-70 |
| | | | | water | |
| 38D | 3C | Industrial waste heat | 30-90 | Hot air | 35-60 |
| 39A | 3C | Industrial waste heat from data centers | 30-90 | Industrial | 110-145 |
| | | | | Steam | |
| 39B | 3C | Industrial waste heat from data centers | 30-90 | Low | 115-130 |
| | | | | Pressure | |
| | | | | Steam | |
| 39C | 3C | Industrial waste heat from data centers | 30-90 | Hot | 50-70 |
| | | | | water | |
| 39D | 3C | Industrial waste heat from data centers | 30-90 | Hot air | 35-60 |
| 40A | 3C | Industrial waste heat from data centers | 30-90 | Industrial | 110-145 |
| | | | | Steam | |
| 40B | 3C | Industrial waste heat from data centers | 30-90 | Low | 115-130 |
| | | | | Pressure | |
| | | | | Steam | |
| 40C | 3C | Industrial waste heat from data centers | 30-90 | Hot | 50-70 |
| | | | | water | |
| 40D | 3C | Industrial waste heat from data centers | 30-90 | Hot air | 35-60 |
| 41A | 3C | Industrial waste heat from beverage | 30-90 | Industrial | 110-145 |
| | | manufacturing and/or bottling | | Steam | |
| 41B | 3C | Industrial waste heat from beverage | 30-90 | Low | 115-130 |
| | | manufacturing and/or bottling | | Pressure | |
| | | | | Steam | |
| 41C | 3C | Industrial waste heat from beverage | 30-90 | Hot | 50-70 |
| | | manufacturing and/or bottling | | water | |
| 41D | 3C | Industrial waste heat from beverage | 30-90 | Hot air | 35-60 |
| | | manufacturing and/or bottling | | | |
| 42A | 3C | Industrial waste heat from beverage | 30-90 | Industrial | 110-145 |
| | | manufacturing and/or bottling | | Steam | |
| 42B | 3C | Industrial waste heat from beverage | 30-90 | Low | 115-130 |
| | | manufacturing and/or bottling | | Pressure | |
| | | | | Steam | |
| 42C | 3C | Industrial waste heat from beverage | 30-90 | Hot | 50-70 |
| | | manufacturing and/or bottling | | water | |
| 42D | 3C | Industrial waste heat from beverage | 30-90 | Hot air | 35-60 |
| | | manufacturing and/or processing | | | |
| 43A | 3C | Industrial waste heat from dairy | 30-90 | Industrial | 110-145 |
| | | manufacturing and/or processing | | Steam | |
| 43B | 3C | Industrial waste heat from dairy | 30-90 | Low | 115-130 |
| | | manufacturing and/or processing | | Pressure | |
| | | | | Steam | |
| 43C | 3C | Industrial waste heat from dairy | 30-90 | Hot | 50-70 |
| | | manufacturing and/or processing | | water | |
| 43D | 3C | Industrial waste heat from dairy | 30-90 | Hot air | 35-60 |
| | | manufacturing and/or processing | | | |
| 44A | 4A1 | Industrial waste heat | 30-90 | Industrial | 110-145 |
| | | | | Steam | |
| 44B | 4A1 | Industrial waste heat | 30-90 | Low | 115-130 |
| | | | | Pressure | |
| | | | | Steam | |
| 44C | 4A1 | Industrial waste heat | 30-90 | Hot | 50-70 |
| | | | | water | |
| 44D | 4A1 | Industrial waste heat | 30-90 | Hot air | 35-60 |
| 45A | 4A1 | Industrial waste heat from data centers | 30-90 | Industrial | 110-145 |
| | | | | Steam | |
| 45B | 4A1 | Industrial waste heat from data centers | 30-90 | Low | 115-130 |
| | | | | Pressure | |
| | | | | Steam | |
| 45C | 4A1 | Industrial waste heat from data centers | 30-90 | Hot | 50-70 |
| | | | | water | |
| 45D | 4A1 | Industrial waste heat from data centers | 30-90 | Hot air | 35-60 |
| 46A | 4A1 | Industrial waste heat from data centers | 30-90 | Industrial | 110-145 |
| | | | | Steam | |
| 46B | 4A1 | Industrial waste heat from data centers | 30-90 | Low | 115-130 |
| | | | | Pressure | |
| | | | | Steam | |
| 46C | 4A1 | Industrial waste heat from data centers | 30-90 | Hot | 50-70 |
| | | | | water | |
| 46D | 4A1 | Industrial waste heat from data centers | 30-90 | Hot air | 35-60 |
| 47A | 4A1 | Industrial waste heat from beverage | 30-90 | Industrial | 110-145 |
| | | manufacturing and/or bottling | | Steam | |
| 47B | 4A1 | Industrial waste heat from beverage | 30-90 | Low | 115-130 |
| | | manufacturing and/or bottling | | Pressure | |
| | | | | Steam | |
| 47C | 4A1 | Industrial waste heat from beverage | 30-90 | Hot | 50-70 |
| | | manufacturing and/or bottling | | water | |
| 47D | 4A1 | Industrial waste heat from beverage | 30-90 | Hot air | 35-60 |
| | | manufacturing and/or bottling | | | |
| 48A | 4A1 | Industrial waste heat from beverage | 30-90 | Industrial | 110-145 |
| | | manufacturing and/or bottling | | Steam | |
| 48B | 4A1 | Industrial waste heat from beverage | 30-90 | Low | 115-130 |
| | | manufacturing and/or bottling | | Pressure | |
| | | | | Steam | |
| 48C | 4A1 | Industrial waste heat from beverage | 30-90 | Hot | 50-70 |
| | | manufacturing and/or bottling | | water | |
| 48D | 4A1 | Industrial waste heat from beverage | 30-90 | Hot air | 35-60 |
| | | manufacturing and/or processing | | | |
| 49A | 4A1 | Industrial waste heat from dairy | 30-90 | Industrial | 110-145 |
| | | manufacturing and/or processing | | Steam | |
| 49B | 4A1 | Industrial waste heat from dairy | 30-90 | Low | 115-130 |
| | | manufacturing and/or processing | | Pressure | |
| | | | | Steam | |
| 49C | 4A1 | Industrial waste heat from dairy | 30-90 | Hot | 50-70 |
| | | manufacturing and/or processing | | water | |
| 49D | 4A1 | Industrial waste heat from dairy | 30-90 | Hot air | 35-60 |
| | | manufacturing and/or processing | | | |
| 50A | 4A2 | Industrial waste heat | 30-90 | Industrial | 110-145 |
| | | | | Steam | |
| 50B | 4A2 | Industrial waste heat | 30-90 | Low | 115-130 |
| | | | | Pressure | |
| | | | | Steam | |
| 50C | 4A2 | Industrial waste heat | 30-90 | Hot | 50-70 |
| | | | | water | |
| 50D | 4A2 | Industrial waste heat | 30-90 | Hot air | 35-60 |
| 51A | 4A2 | Industrial waste heat from data centers | 30-90 | Industrial | 110-145 |
| | | | | Steam | |
| 51B | 4A2 | Industrial waste heat from data centers | 30-90 | Low | 115-130 |
| | | | | Pressure | |
| | | | | Steam | |
| 51C | 4A2 | Industrial waste heat from data centers | 30-90 | Hot | 50-70 |
| | | | | water | |
| 51D | 4A2 | Industrial waste heat from data centers | 30-90 | Hot air | 35-60 |
| 52A | 4A2 | Industrial waste heat from data centers | 30-90 | Industrial | 110-145 |
| | | | | Steam | |
| 52B | 4A2 | Industrial waste heat from data centers | 30-90 | Low | 115-130 |
| | | | | Pressure | |
| | | | | Steam | |
| 52C | 4A2 | Industrial waste heat from data centers | 30-90 | Hot | 50-70 |
| | | | | water | |
| 52D | 4A2 | Industrial waste heat from data centers | 30-90 | Hot air | 35-60 |
| 53A | 4A2 | Industrial waste heat from beverage | 30-90 | Industrial | 110-145 |
| | | manufacturing and/or bottling | | Steam | |
| 53B | 4A2 | Industrial waste heat from beverage | 30-90 | Low | 115-130 |
| | | manufacturing and/or bottling | | Pressure | |
| | | | | Steam | |
| 53C | 4A2 | Industrial waste heat from beverage | 30-90 | Hot | 50-70 |
| | | manufacturing and/or bottling | | water | |
| 53D | 4A2 | Industrial waste heat from beverage | 30-90 | Hot air | 35-60 |
| | | manufacturing and/or bottling | | | |
| 54A | 4A2 | Industrial waste heat from beverage | 30-90 | Industrial | 110-145 |
| | | manufacturing and/or bottling | | Steam | |
| 54B | 4A2 | Industrial waste heat from beverage | 30-90 | Low | 115-130 |
| | | manufacturing and/or bottling | | Pressure | |
| | | | | Steam | |
| 54C | 4A2 | Industrial waste heat from beverage | 30-90 | Hot | 50-70 |
| | | manufacturing and/or bottling | | water | |
| 54D | 4A2 | Industrial waste heat from beverage | 30-90 | Hot air | 35-60 |
| | | manufacturing and/or processing | | | |
| 55A | 4A2 | Industrial waste heat from dairy | 30-90 | Industrial | 110-145 |
| | | manufacturing and/or processing | | Steam | |
| 55B | 4A2 | Industrial waste heat from dairy | 30-90 | Low | 115-130 |
| | | manufacturing and/or processing | | Pressure | |
| | | | | Steam | |
| 55C | 4A2 | Industrial waste heat from dairy | 30-90 | Hot | 50-70 |
| | | manufacturing and/or processing | | water | |
| 55D | 4A2 | Industrial waste heat from dairy | 30-90 | Hot air | 35-60 |
| | | manufacturing and/or processing | | | |
| 56A | 4A3 | Industrial waste heat | 30-90 | Industrial | 110-145 |
| | | | | Steam | |
| 56B | 4A3 | Industrial waste heat | 30-90 | Low | 115-130 |
| | | | | Pressure | |
| | | | | Steam | |
| 56C | 4A3 | Industrial waste heat | 30-90 | Hot | 50-70 |
| | | | | water | |
| 56D | 4A3 | Industrial waste heat | 30-90 | Hot air | 35-60 |
| 57A | 4A3 | Industrial waste heat from data centers | 30-90 | Industrial | 110-145 |
| | | | | Steam | |
| 57B | 4A3 | Industrial waste heat from data centers | 30-90 | Low | 115-130 |
| | | | | Pressure | |
| | | | | Steam | |
| 57C | 4A3 | Industrial waste heat from data centers | 30-90 | Hot | 50-70 |
| | | | | water | |
| 57D | 4A3 | Industrial waste heat from data centers | 30-90 | Hot air | 35-60 |
| 58A | 4A3 | Industrial waste heat from data centers | 30-90 | Industrial | 110-145 |
| | | | | Steam | |
| 58B | 4A3 | Industrial waste heat from data centers | 30-90 | Low | 115-130 |
| | | | | Pressure | |
| | | | | Steam | |
| 58C | 4A3 | Industrial waste heat from data centers | 30-90 | Hot | 50-70 |
| | | | | water | |
| 58D | 4A3 | Industrial waste heat from data centers | 30-90 | Hot air | 35-60 |
| 59A | 4A3 | Industrial waste heat from beverage | 30-90 | Industrial | 110-145 |
| | | manufacturing and/or bottling | | Steam | |
| 59B | 4A3 | Industrial waste heat from beverage | 30-90 | Low | 115-130 |
| | | manufacturing and/or bottling | | Pressure | |
| | | | | Steam | |
| 59C | 4A3 | Industrial waste heat from beverage | 30-90 | Hot | 50-70 |
| | | manufacturing and/or bottling | | water | |
| 59D | 4A3 | Industrial waste heat from beverage | 30-90 | Hot air | 35-60 |
| | | manufacturing and/or bottling | | | |
| 60A | 4A3 | Industrial waste heat from beverage | 30-90 | Industrial | 110-145 |
| | | manufacturing and/or bottling | | Steam | |
| 60B | 4A3 | Industrial waste heat from beverage | 30-90 | Low | 115-130 |
| | | manufacturing and/or bottling | | Pressure | |
| | | | | Steam | |
| 60C | 4A3 | Industrial waste heat from beverage | 30-90 | Hot | 50-70 |
| | | manufacturing and/or bottling | | water | |
| 60D | 4A3 | Industrial waste heat from beverage | 30-90 | Hot air | 35-60 |
| | | manufacturing and/or processing | | | |
| 61A | 4A3 | Industrial waste heat from dairy | 30-90 | Industrial | 110-145 |
| | | manufacturing and/or processing | | Steam | |
| 61B | 4A3 | Industrial waste heat from dairy | 30-90 | Low | 115-130 |
| | | manufacturing and/or processing | | Pressure | |
| | | | | Steam | |
| 61C | 4A3 | Industrial waste heat from dairy | 30-90 | Hot | 50-70 |
| | | manufacturing and/or processing | | water | |
| 61D | 4A3 | Industrial waste heat from dairy | 30-90 | Hot air | 35-60 |
| | | manufacturing and/or processing | | | |
| 62A | 4B1 | Industrial waste heat | 30-90 | Industrial | 110-145 |
| | | | | Steam | |
| 62B | 4B1 | Industrial waste heat | 30-90 | Low | 115-130 |
| | | | | Pressure | |
| | | | | Steam | |
| 62C | 4B1 | Industrial waste heat | 30-90 | Hot | 50-70 |
| | | | | water | |
| 62D | 4B1 | Industrial waste heat | 30-90 | Hot air | 35-60 |
| 63A | 4B1 | Industrial waste heat from data centers | 30-90 | Industrial | 110-145 |
| | | | | Steam | |
| 63B | 4B1 | Industrial waste heat from data centers | 30-90 | Low | 115-130 |
| | | | | Pressure | |
| | | | | Steam | |
| 63C | 4B1 | Industrial waste heat from data centers | 30-90 | Hot | 50-70 |
| | | | | water | |
| 63D | 4B1 | Industrial waste heat from data centers | 30-90 | Hot air | 35-60 |
| 64A | 4B1 | Industrial waste heat from data centers | 30-90 | Industrial | 110-145 |
| | | | | Steam | |
| 64B | 4B1 | Industrial waste heat from data centers | 30-90 | Low | 115-130 |
| | | | | Pressure | |
| | | | | Steam | |
| 64C | 4B1 | Industrial waste heat from data centers | 30-90 | Hot | 50-70 |
| | | | | water | |
| 64D | 4B1 | Industrial waste heat from data centers | 30-90 | Hot air | 35-60 |
| 65A | 4B1 | Industrial waste heat from beverage | 30-90 | Industrial | 110-145 |
| | | manufacturing and/or bottling | | Steam | |
| 65B | 4B1 | Industrial waste heat from beverage | 30-90 | Low | 115-130 |
| | | manufacturing and/or bottling | | Pressure | |
| | | | | Steam | |
| 65C | 4B1 | Industrial waste heat from beverage | 30-90 | Hot | 50-70 |
| | | manufacturing and/or bottling | | water | |
| 65D | 4B1 | Industrial waste heat from beverage | 30-90 | Hot air | 35-60 |
| | | manufacturing and/or bottling | | | |
| 66A | 4B1 | Industrial waste heat from beverage | 30-90 | Industrial | 110-145 |
| | | manufacturing and/or bottling | | Steam | |
| 66B | 4B1 | Industrial waste heat from beverage | 30-90 | Low | 115-130 |
| | | manufacturing and/or bottling | | Pressure | |
| | | | | Steam | |
| 66C | 4B1 | Industrial waste heat from beverage | 30-90 | Hot | 50-70 |
| | | manufacturing and/or bottling | | water | |
| 66D | 4B1 | Industrial waste heat from beverage | 30-90 | Hot air | 35-60 |
| | | manufacturing and/or processing | | | |
| 67A | 4B1 | Industrial waste heat from dairy | 30-90 | Industrial | 110-145 |
| | | manufacturing and/or processing | | Steam | |
| 67B | 4B1 | Industrial waste heat from dairy | 30-90 | Low | 115-130 |
| | | manufacturing and/or processing | | Pressure | |
| | | | | Steam | |
| 67C | 4B1 | Industrial waste heat from dairy | 30-90 | Hot | 50-70 |
| | | manufacturing and/or processing | | water | |
| 67D | 4B1 | Industrial waste heat from dairy | 30-90 | Hot air | 35-60 |
| | | manufacturing and/or processing | | | |
| 68A | 4B2 | Industrial waste heat | 30-90 | Industrial | 110-145 |
| | | | | Steam | |
| 68B | 4B2 | Industrial waste heat | 30-90 | Low | 115-130 |
| | | | | Pressure | |
| | | | | Steam | |
| 68C | 4B2 | Industrial waste heat | 30-90 | Hot | 50-70 |
| | | | | water | |
| 68D | 4B2 | Industrial waste heat | 30-90 | Hot air | 35-60 |
| 69A | 4B2 | Industrial waste heat from data centers | 30-90 | Industrial | 110-145 |
| | | | | Steam | |
| 69B | 4B2 | Industrial waste heat from data centers | 30-90 | Low | 115-130 |
| | | | | Pressure | |
| | | | | Steam | |
| 69C | 4B2 | Industrial waste heat from data centers | 30-90 | Hot | 50-70 |
| | | | | water | |
| 69D | 4B2 | Industrial waste heat from data centers | 30-90 | Hot air | 35-60 |
| 70A | 4B2 | Industrial waste heat from data centers | 30-90 | Industrial | 110-145 |
| | | | | Steam | |
| 70B | 4B2 | Industrial waste heat from data centers | 30-90 | Low | 115-130 |
| | | | | Pressure | |
| | | | | Steam | |
| 70C | 4B2 | Industrial waste heat from data centers | 30-90 | Hot | 50-70 |
| | | | | water | |
| 70D | 4B2 | Industrial waste heat from data centers | 30-90 | Hot air | 35-60 |
| 71A | 4B2 | Industrial waste heat from beverage | 30-90 | Industrial | 110-145 |
| | | manufacturing and/or bottling | | Steam | |
| 71B | 4B2 | Industrial waste heat from beverage | 30-90 | Low | 115-130 |
| | | manufacturing and/or bottling | | Pressure | |
| | | | | Steam | |
| 71C | 4B2 | Industrial waste heat from beverage | 30-90 | Hot | 50-70 |
| | | manufacturing and/or bottling | | water | |
| 71D | 4B2 | Industrial waste heat from beverage | 30-90 | Hot air | 35-60 |
| | | manufacturing and/or bottling | | | |
| 72A | 4B2 | Industrial waste heat from beverage | 30-90 | Industrial | 110-145 |
| | | manufacturing and/or bottling | | Steam | |
| 72B | 4B2 | Industrial waste heat from beverage | 30-90 | Low | 115-130 |
| | | manufacturing and/or bottling | | Pressure | |
| | | | | Steam | |
| 72C | 4B2 | Industrial waste heat from beverage | 30-90 | Hot | 50-70 |
| | | manufacturing and/or bottling | | water | |
| 72D | 4B2 | Industrial waste heat from beverage | 30-90 | Hot air | 35-60 |
| | | manufacturing and/or processing | | | |
| 73A | 4B3 | Industrial waste heat from dairy | 30-90 | Industrial | 110-145 |
| | | manufacturing and/or processing | | Steam | |
| 73B | 4B3 | Industrial waste heat from dairy | 30-90 | Low | 115-130 |
| | | manufacturing and/or processing | | Pressure | |
| | | | | Steam | |
| 73C | 4B3 | Industrial waste heat from dairy | 30-90 | Hot | 50-70 |
| | | manufacturing and/or processing | | water | |
| 73D | 4B3 | Industrial waste heat from dairy | 30-90 | Hot air | 35-60 |
| | | manufacturing and/or processing | | | |
| 74A | 4B3 | Industrial waste heat | 30-90 | Industrial | 110-145 |
| | | | | Steam | |
| 74B | 4B3 | Industrial waste heat | 30-90 | Low | 115-130 |
| | | | | Pressure | |
| | | | | Steam | |
| 74C | 4B3 | Industrial waste heat | 30-90 | Hot | 50-70 |
| | | | | water | |
| 74D | 4B3 | Industrial waste heat | 30-90 | Hot air | 35-60 |
| 75A | 4B3 | Industrial waste heat from data centers | 30-90 | Industrial | 110-145 |
| | | | | Steam | |
| 75B | 4B3 | Industrial waste heat from data centers | 30-90 | Low | 115-130 |
| | | | | Pressure | |
| | | | | Steam | |
| 75C | 4B3 | Industrial waste heat from data centers | 30-90 | Hot | 50-70 |
| | | | | water | |
| 75D | 4B3 | Industrial waste heat from data centers | 30-90 | Hot air | 35-60 |
| 76A | 4B3 | Industrial waste heat from data centers | 30-90 | Industrial | 110-145 |
| | | | | Steam | |
| 76B | 4B3 | Industrial waste heat from data centers | 30-90 | Low | 115-130 |
| | | | | Pressure | |
| | | | | Steam | |
| 76C | 4B3 | Industrial waste heat from data centers | 30-90 | Hot | 50-70 |
| | | | | water | |
| 76D | 4B3 | Industrial waste heat from data centers | 30-90 | Hot air | 35-60 |
| 77A | 4B3 | Industrial waste heat from beverage | 30-90 | Industrial | 110-145 |
| | | manufacturing and/or bottling | | Steam | |
| 77B | 4B3 | Industrial waste heat from beverage | 30-90 | Low | 115-130 |
| | | manufacturing and/or bottling | | Pressure | |
| | | | | Steam | |
| 77C | 4B3 | Industrial waste heat from beverage | 30-90 | Hot | 50-70 |
| | | manufacturing and/or bottling | | water | |
| 77D | 4B3 | Industrial waste heat from beverage | 30-90 | Hot air | 35-60 |
| | | manufacturing and/or bottling | | | |
| 78A | 4B3 | Industrial waste heat from beverage | 30-90 | Industrial | 110-145 |
| | | manufacturing and/or bottling | | Steam | |
| 78B | 4B3 | Industrial waste heat from beverage | 30-90 | Low | 115-130 |
| | | manufacturing and/or bottling | | Pressure | |
| | | | | Steam | |
| 78C | 4B3 | Industrial waste heat from beverage | 30-90 | Hot | 50-70 |
| | | manufacturing and/or bottling | | water | |
| 78D | 4B3 | Industrial waste heat from beverage | 30-90 | Hot air | 35-60 |
| | | manufacturing and/or processing | | | |
| 79A | 4B3 | Industrial waste heat from dairy | 30-90 | Industrial | 110-145 |
| | | manufacturing and/or processing | | Steam | |
| 79B | 4B3 | Industrial waste heat from dairy | 30-90 | Low | 115-130 |
| | | manufacturing and/or processing | | Pressure | |
| | | | | Steam | |
| 79C | 4B3 | Industrial waste heat from dairy | 30-90 | Hot | 50-70 |
| | | manufacturing and/or processing | | water | |
| 79D | 4B3 | Industrial waste heat from dairy | 30-90 | Hot air | 35-60 |
| | | manufacturing and/or processing | | | |
| 80A | 4C1 | Industrial waste heat from dairy | 30-90 | Industrial | 110-145 |
| | | manufacturing and/or processing | | Steam | |
| 80B | 4C1 | Industrial waste heat from dairy | 30-90 | Low | 115-130 |
| | | manufacturing and/or processing | | Pressure | |
| | | | | Steam | |
| 80C | 4C1 | Industrial waste heat from dairy | 30-90 | Hot | 50-70 |
| | | manufacturing and/or processing | | water | |
| 80D | 4C1 | Industrial waste heat from dairy | 30-90 | Hot air | 35-60 |
| | | manufacturing and/or processing | | | |
| 81A | 4C1 | Industrial waste heat | 30-90 | Industrial | 110-145 |
| | | | | Steam | |
| 81B | 4C1 | Industrial waste heat | 30-90 | Low | 115-130 |
| | | | | Pressure | |
| | | | | Steam | |
| 81C | 4C1 | Industrial waste heat | 30-90 | Hot | 50-70 |
| | | | | water | |
| 81D | 4C1 | Industrial waste heat | 30-90 | Hot air | 35-60 |
| 82A | 4C1 | Industrial waste heat from data centers | 30-90 | Industrial | 110-145 |
| | | | | Steam | |
| 82B | 4C1 | Industrial waste heat from data centers | 30-90 | Low | 115-130 |
| | | | | Pressure | |
| | | | | Steam | |
| 82C | 4C1 | Industrial waste heat from data centers | 30-90 | Hot | 50-70 |
| | | | | water | |
| 83D | 4C1 | Industrial waste heat from data centers | 30-90 | Hot air | 35-60 |
| 84A | 4C1 | Industrial waste heat from data centers | 30-90 | Industrial | 110-145 |
| | | | | Steam | |
| 84B | 4C1 | Industrial waste heat from data centers | 30-90 | Low | 115-130 |
| | | | | Pressure | |
| | | | | Steam | |
| 84C | 4C1 | Industrial waste heat from data centers | 30-90 | Hot | 50-70 |
| | | | | water | |
| 84D | 4C1 | Industrial waste heat from data centers | 30-90 | Hot air | 35-60 |
| 85A | 4C1 | Industrial waste heat from beverage | 30-90 | Industrial | 110-145 |
| | | manufacturing and/or bottling | | Steam | |
| 85B | 4C1 | Industrial waste heat from beverage | 30-90 | Low | 115-130 |
| | | manufacturing and/or bottling | | Pressure | |
| | | | | Steam | |
| 85C | 4C1 | Industrial waste heat from beverage | 30-90 | Hot | 50-70 |
| | | manufacturing and/or bottling | | water | |
| 85D | 4C1 | Industrial waste heat from beverage | 30-90 | Hot air | 35-60 |
| | | manufacturing and/or bottling | | | |
| 86A | 4C1 | Industrial waste heat from beverage | 30-90 | Industrial | 110-145 |
| | | manufacturing and/or bottling | | Steam | |
| 86A | 4C1 | Industrial waste heat from beverage | 30-90 | Low | 115-130 |
| | | manufacturing and/or bottling | | Pressure | |
| | | | | Steam | |
| 86B | 4C1 | Industrial waste heat from data centers | 30-90 | Low | 115-130 |
| | | | | Pressure | |
| | | | | Steam | |
| 86C | 4C1 | Industrial waste heat from beverage | 30-90 | Hot | 50-70 |
| | | manufacturing and/or bottling | | water | |
| 86D | 4C1 | Industrial waste heat from beverage | 30-90 | Hot air | 35-60 |
| | | manufacturing and/or processing | | | |
| 87A | 4C1 | Industrial waste heat from dairy | 30-90 | Industrial | 110-145 |
| | | manufacturing and/or processing | | Steam | |
| 87B | 4C1 | Industrial waste heat from dairy | 30-90 | Low | 115-130 |
| | | manufacturing and/or processing | | Pressure | |
| | | | | Steam | |
| 87C | 4C1 | Industrial waste heat from dairy | 30-90 | Hot | 50-70 |
| | | manufacturing and/or processing | | water | |
| 87D | 4C1 | Industrial waste heat from dairy | 30-90 | Hot air | 35-60 |
| | | manufacturing and/or processing | | | |
| 88A | 4C2 | Industrial waste heat from dairy | 30-90 | Industrial | 110-145 |
| | | manufacturing and/or processing | | Steam | |
| 88B | 4C2 | Industrial waste heat from dairy | 30-90 | Low | 115-130 |
| | | manufacturing and/or processing | | Pressure | |
| | | | | Steam | |
| 88C | 4C2 | Industrial waste heat from dairy | 30-90 | Hot | 50-70 |
| | | manufacturing and/or processing | | water | |
| 88D | 4C2 | Industrial waste heat from dairy | 30-90 | Hot air | 35-60 |
| | | manufacturing and/or processing | | | |
| 89A | 4C2 | Industrial waste heat | 30-90 | Industrial | 110-145 |
| | | | | Steam | |
| 89B | 4C2 | Industrial waste heat | 30-90 | Low | 115-130 |
| | | | | Pressure | |
| | | | | Steam | |
| 89C | 4C2 | Industrial waste heat | 30-90 | Hot | 50-70 |
| | | | | water | |
| 89D | 4C2 | Industrial waste heat | 30-90 | Hot air | 35-60 |
| 90A | 4C2 | Industrial waste heat from data centers | 30-90 | Industrial | 110-145 |
| | | | | Steam | |
| 90B | 4C2 | Industrial waste heat from data centers | 30-90 | Low | 115-130 |
| | | | | Pressure | |
| | | | | Steam | |
| 90C | 4C2 | Industrial waste heat from data centers | 30-90 | Hot | 50-70 |
| | | | | water | |
| 90D | 4C2 | Industrial waste heat from data centers | 30-90 | Hot air | 35-60 |
| 91A | 4C2 | Industrial waste heat from data centers | 30-90 | Industrial | 110-145 |
| | | | | Steam | |
| 91B | 4C2 | Industrial waste heat from data centers | 30-90 | Low | 115-130 |
| | | | | Pressure | |
| | | | | Steam | |
| 91C | 4C2 | Industrial waste heat from data centers | 30-90 | Hot | 50-70 |
| | | | | water | |
| 91D | 4C2 | Industrial waste heat from data centers | 30-90 | Hot air | 35-60 |
| 92A | 4C2 | Industrial waste heat from beverage | 30-90 | Industrial | 110-145 |
| | | manufacturing and/or bottling | | Steam | |
| 92B | 4C2 | Industrial waste heat from beverage | 30-90 | Low | 115-130 |
| | | manufacturing and/or bottling | | Pressure | |
| | | | | Steam | |
| 92C | 4C2 | Industrial waste heat from beverage | 30-90 | Hot | 50-70 |
| | | manufacturing and/or bottling | | water | |
| 92D | 4C2 | Industrial waste heat from beverage | 30-90 | Hot air | 35-60 |
| | | manufacturing and/or bottling | | | |
| 93A | 4C2 | Industrial waste heat from beverage | 30-90 | Industrial | 110-145 |
| | | manufacturing and/or bottling | | Steam | |
| 93B | 4C2 | Industrial waste heat from beverage | 30-90 | Low | 115-130 |
| | | manufacturing and/or bottling | | Pressure | |
| | | | | Steam | |
| 93B | 4C2 | Industrial waste heat from data centers | 30-90 | Low | 115-130 |
| | | | | Pressure | |
| | | | | Steam | |
| 93C | 4C2 | Industrial waste heat from beverage | 30-90 | Hot | 50-70 |
| | | manufacturing and/or bottling | | water | |
| 93D | 4C2 | Industrial waste heat from beverage | 30-90 | Hot air | 35-60 |
| | | manufacturing and/or processing | | | |
| 94A | 4C2 | Industrial waste heat from dairy | 30-90 | Industrial | 110-145 |
| | | manufacturing and/or processing | | Steam | |
| 94B | 4C2 | Industrial waste heat from dairy | 30-90 | Low | 115-130 |
| | | manufacturing and/or processing | | Pressure | |
| | | | | Steam | |
| 94C | 4C2 | Industrial waste heat from dairy | 30-90 | Hot | 50-70 |
| | | manufacturing and/or processing | | water | |
| 94D | 4C2 | Industrial waste heat from dairy | 30-90 | Hot air | 35-60 |
| | | manufacturing and/or processing | | | |
| 95A | 4C3 | Industrial waste heat from dairy | 30-90 | Industrial | 110-145 |
| | | manufacturing and/or processing | | Steam | |
| 95B | 4C3 | Industrial waste heat from dairy | 30-90 | Low | 115-130 |
| | | manufacturing and/or processing | | Pressure | |
| | | | | Steam | |
| 95C | 4C3 | Industrial waste heat from dairy | 30-90 | Hot | 50-70 |
| | | manufacturing and/or processing | | water | |
| 95D | 4C3 | Industrial waste heat from dairy | 30-90 | Hot air | 35-60 |
| | | manufacturing and/or processing | | | |
| 96A | 4C3 | Industrial waste heat | 30-90 | Industrial | 110-145 |
| | | | | Steam | |
| 96B | 4C3 | Industrial waste heat | 30-90 | Low | 115-130 |
| | | | | Pressure | |
| | | | | Steam | |
| 96C | 4C3 | Industrial waste heat | 30-90 | Hot | 50-70 |
| | | | | water | |
| 96D | 4C3 | Industrial waste heat | 30-90 | Hot air | 35-60 |
| 97A | 4C3 | Industrial waste heat from data centers | 30-90 | Industrial | 110-145 |
| | | | | Steam | |
| 97B | 4C3 | Industrial waste heat from data centers | 30-90 | Low | 115-130 |
| | | | | Pressure | |
| | | | | Steam | |
| 97C | 4C3 | Industrial waste heat from data centers | 30-90 | Hot | 50-70 |
| | | | | water | |
| 97D | 4C3 | Industrial waste heat from data centers | 30-90 | Hot air | 35-60 |
| 98A | 4C3 | Industrial waste heat from data centers | 30-90 | Industrial | 110-145 |
| | | | | Steam | |
| 98B | 4C3 | Industrial waste heat from data centers | 30-90 | Low | 115-130 |
| | | | | Pressure | |
| | | | | Steam | |
| 98C | 4C3 | Industrial waste heat from data centers | 30-90 | Hot | 50-70 |
| | | | | water | |
| 98D | 4C3 | Industrial waste heat from data centers | 30-90 | Hot air | 35-60 |
| 99A | 4C3 | Industrial waste heat from beverage | 30-90 | Industrial | 110-145 |
| | | manufacturing and/or bottling | | Steam | |
| 99B | 4C3 | Industrial waste heat from beverage | 30-90 | Low | 115-130 |
| | | manufacturing and/or bottling | | Pressure | |
| | | | | Steam | |
| 99C | 4C3 | Industrial waste heat from beverage | 30-90 | Hot | 50-70 |
| | | manufacturing and/or bottling | | water | |
| 99D | 4C3 | Industrial waste heat from beverage | 30-90 | Hot air | 35-60 |
| | | manufacturing and/or bottling | | | |
| 100A | 4C3 | Industrial waste heat from beverage | 30-90 | Industrial | 110-145 |
| | | manufacturing and/or bottling | | Steam | |
| 100B | 4C3 | Industrial waste heat from data centers | 30-90 | Low | 115-130 |
| | | | | Pressure | |
| | | | | Steam | |
| 100C | 4C3 | Industrial waste heat from beverage | 30-90 | Hot | 50-70 |
| | | manufacturing and/or bottling | | water | |
| 100D | 4C3 | Industrial waste heat from beverage | 30-90 | Hot air | 35-60 |
| | | manufacturing and/or processing | | | |
| 101A | 4C3 | Industrial waste heat from dairy | 30-90 | Industrial | 110-145 |
| | | manufacturing and/or processing | | Steam | |
| 101B | 4C3 | Industrial waste heat from dairy | 30-90 | Low | 115-130 |
| | | manufacturing and/or processing | | Pressure | |
| | | | | Steam | |
| 101C | 4C3 | Industrial waste heat from dairy | 30-90 | Hot | 50-70 |
| | | manufacturing and/or processing | | water | |
| 101D | 4C3 | Industrial waste heat from dairy | 30-90 | Hot air | 35-60 |
| | | manufacturing and/or processing | | | |
| 102A | 5A | Industrial waste heat from dairy | 30-90 | Industrial | 110-145 |
| | | manufacturing and/or processing | | Steam | |
| 102B | 5A | Industrial waste heat from dairy | 30-90 | Low | 115-130 |
| | | manufacturing and/or processing | | Pressure | |
| | | | | Steam | |
| 102C | 5A | Industrial waste heat from dairy | 30-90 | Hot | 50-70 |
| | | manufacturing and/or processing | | water | |
| 102D | 5A | Industrial waste heat from dairy | 30-90 | Hot air | 35-60 |
| | | manufacturing and/or processing | | | |
| 103A | 5A | Industrial waste heat | 30-90 | Industrial | 110-145 |
| | | | | Steam | |
| 103B | 5A | Industrial waste heat | 30-90 | Low | 115-130 |
| | | | | Pressure | |
| | | | | Steam | |
| 103C | 5A | Industrial waste heat | 30-90 | Hot | 50-70 |
| | | | | water | |
| 103D | 5A | Industrial waste heat | 30-90 | Hot air | 35-60 |
| 104A | 5A | Industrial waste heat from data centers | 30-90 | Industrial | 110-145 |
| | | | | Steam | |
| 104B | 5A | Industrial waste heat from data centers | 30-90 | Low | 115-130 |
| | | | | Pressure | |
| | | | | Steam | |
| 104C | 5A | Industrial waste heat from data centers | 30-90 | Hot | 50-70 |
| | | | | water | |
| 104D | 5A | Industrial waste heat from data centers | 30-90 | Hot air | 35-60 |
| 105A | 5A | Industrial waste heat from data centers | 30-90 | Industrial | 110-145 |
| | | | | Steam | |
| 105B | 5A | Industrial waste heat from data centers | 30-90 | Low | 115-130 |
| | | | | Pressure | |
| | | | | Steam | |
| 105C | 5A | Industrial waste heat from data centers | 30-90 | Hot | 50-70 |
| | | | | water | |
| 105D | 5A | Industrial waste heat from data centers | 30-90 | Hot air | 35-60 |
| 106A | 5A | Industrial waste heat from beverage | 30-90 | Industrial | 110-145 |
| | | manufacturing and/or bottling | | Steam | |
| 106B | 5A | Industrial waste heat from beverage | 30-90 | Low | 115-130 |
| | | manufacturing and/or bottling | | Pressure | |
| | | | | Steam | |
| 106C | 5A | Industrial waste heat from beverage | 30-90 | Hot | 50-70 |
| | | manufacturing and/or bottling | | water | |
| 106D | 5A | Industrial waste heat from beverage | 30-90 | Hot air | 35-60 |
| | | manufacturing and/or bottling | | | |
| 107A | 5A | Industrial waste heat from beverage | 30-90 | Industrial | 110-145 |
| | | manufacturing and/or bottling | | Steam | |
| 107B | 5A | Industrial waste heat from data centers | 30-90 | Low | 115-130 |
| | | | | Pressure | |
| | | | | Steam | |
| 107C | 5A | Industrial waste heat from beverage | 30-90 | Hot | 50-70 |
| | | manufacturing and/or bottling | | water | |
| 107D | 5A | Industrial waste heat from beverage | 30-90 | Hot air | 35-60 |
| | | manufacturing and/or processing | | | |
| 108A | 5A | Industrial waste heat from dairy | 30-90 | Industrial | 110-145 |
| | | manufacturing and/or processing | | Steam | |
| 108B | 5A | Industrial waste heat from dairy | 30-90 | Low | 115-130 |
| | | manufacturing and/or processing | | Pressure | |
| | | | | Steam | |
| 108C | 5A | Industrial waste heat from dairy | 30-90 | Hot | 50-70 |
| | | manufacturing and/or processing | | water | |
| 108D | 5A | Industrial waste heat from dairy | 30-90 | Hot air | 35-60 |
| | | manufacturing and/or processing | | | |
| 109A | 5B | Industrial waste heat from dairy | 30-90 | Industrial | 110-145 |
| | | manufacturing and/or processing | | Steam | |
| 109B | 5B | Industrial waste heat from dairy | 30-90 | Low | 115-130 |
| | | manufacturing and/or processing | | Pressure | |
| | | | | Steam | |
| 109C | 5B | Industrial waste heat from dairy | 30-90 | Hot | 50-70 |
| | | manufacturing and/or processing | | water | |
| 109D | 5B | Industrial waste heat from dairy | 30-90 | Hot air | 35-60 |
| | | manufacturing and/or processing | | | |
| 110A | 5B | Industrial waste heat | 30-90 | Industrial | 110-145 |
| | | | | Steam | |
| 110B | 5B | Industrial waste heat | 30-90 | Low | 115-130 |
| | | | | Pressure | |
| | | | | Steam | |
| 110C | 5B | Industrial waste heat | 30-90 | Hot | 50-70 |
| | | | | water | |
| 110D | 5B | Industrial waste heat | 30-90 | Hot air | 35-60 |
| 111A | 5B | Industrial waste heat from data centers | 30-90 | Industrial | 110-145 |
| | | | | Steam | |
| 111B | 5B | Industrial waste heat from data centers | 30-90 | Low | 115-130 |
| | | | | Pressure | |
| | | | | Steam | |
| 111C | 5B | Industrial waste heat from data centers | 30-90 | Hot | 50-70 |
| | | | | water | |
| 111D | 5B | Industrial waste heat from data centers | 30-90 | Hot air | 35-60 |
| 112A | 5B | Industrial waste heat from data centers | 30-90 | Industrial | 110-145 |
| | | | | Steam | |
| 112B | 5B | Industrial waste heat from data centers | 30-90 | Low | 115-130 |
| | | | | Pressure | |
| | | | | Steam | |
| 112C | 5B | Industrial waste heat from data centers | 30-90 | Hot | 50-70 |
| | | | | water | |
| 112D | 5B | Industrial waste heat from data centers | 30-90 | Hot air | 35-60 |
| 113A | 5B | Industrial waste heat from beverage | 30-90 | Industrial | 110-145 |
| | | manufacturing and/or bottling | | Steam | |
| 113B | 5B | Industrial waste heat from beverage | 30-90 | Low | 115-130 |
| | | manufacturing and/or bottling | | Pressure | |
| | | | | Steam | |
| 113C | 5B | Industrial waste heat from beverage | 30-90 | Hot | 50-70 |
| | | manufacturing and/or bottling | | water | |
| 113D | 5B | Industrial waste heat from beverage | 30-90 | Hot air | 35-60 |
| | | manufacturing and/or bottling | | | |
| 114A | 5B | Industrial waste heat from beverage | 30-90 | Industrial | 110-145 |
| | | manufacturing and/or bottling | | Steam | |
| 114B | 5B | Industrial waste heat from data centers | 30-90 | Low | 115-130 |
| | | | | Pressure | |
| | | | | Steam | |
| 114C | 5B | Industrial waste heat from beverage | 30-90 | Hot | 50-70 |
| | | manufacturing and/or bottling | | water | |
| 114D | 5B | Industrial waste heat from beverage | 30-90 | Hot air | 35-60 |
| | | manufacturing and/or processing | | | |
| 115A | 5B | Industrial waste heat from dairy | 30-90 | Industrial | 110-145 |
| | | manufacturing and/or processing | | Steam | |
| 115B | 5B | Industrial waste heat from dairy | 30-90 | Low | 115-130 |
| | | manufacturing and/or processing | | Pressure | |
| | | | | Steam | |
| 115C | 5B | Industrial waste heat from dairy | 30-90 | Hot | 50-70 |
| | | manufacturing and/or processing | | water | |
| 115D | 5B | Industrial waste heat from dairy | 30-90 | Hot air | 35-60 |
| | | manufacturing and/or processing | | | |
| 116A | 5C | Industrial waste heat from dairy | 30-90 | Industrial | 110-145 |
| | | manufacturing and/or processing | | Steam | |
| 116B | 5C | Industrial waste heat from dairy | 30-90 | Low | 115-130 |
| | | manufacturing and/or processing | | Pressure | |
| | | | | Steam | |
| 116C | 5C | Industrial waste heat from dairy | 30-90 | Hot | 50-70 |
| | | manufacturing and/or processing | | water | |
| 116D | 5C | Industrial waste heat from dairy | 30-90 | Hot air | 35-60 |
| | | manufacturing and/or processing | | | |
| 117A | 5C | Industrial waste heat | 30-90 | Industrial | 110-145 |
| | | | | Steam | |
| 117B | 5C | Industrial waste heat | 30-90 | Low | 115-130 |
| | | | | Pressure | |
| | | | | Steam | |
| 117C | 5C | Industrial waste heat | 30-90 | Hot | 50-70 |
| | | | | water | |
| 117D | 5C | Industrial waste heat | 30-90 | Hot air | 35-60 |
| 118A | 5C | Industrial waste heat from data centers | 30-90 | Industrial | 110-145 |
| | | | | Steam | |
| 118B | 5C | Industrial waste heat from data centers | 30-90 | Low | 115-130 |
| | | | | Pressure | |
| | | | | Steam | |
| 118C | 5C | Industrial waste heat from data centers | 30-90 | Hot | 50-70 |
| | | | | water | |
| 118D | 5C | Industrial waste heat from data centers | 30-90 | Hot air | 35-60 |
| 119A | 5C | Industrial waste heat from data centers | 30-90 | Industrial | 110-145 |
| | | | | Steam | |
| 119B | 5C | Industrial waste heat from data centers | 30-90 | Low | 115-130 |
| | | | | Pressure | |
| | | | | Steam | |
| 119C | 5C | Industrial waste heat from data centers | 30-90 | Hot | 50-70 |
| | | | | water | |
| 119D | 5C | Industrial waste heat from data centers | 30-90 | Hot air | 35-60 |
| 120A | 5C | Industrial waste heat from beverage | 30-90 | Industrial | 110-145 |
| | | manufacturing and/or bottling | | Steam | |
| 120B | 5C | Industrial waste heat from beverage | 30-90 | Low | 115-130 |
| | | manufacturing and/or bottling | | Pressure | |
| | | | | Steam | |
| 120C | 5C | Industrial waste heat from beverage | 30-90 | Hot | 50-70 |
| | | manufacturing and/or bottling | | water | |
| 120D | 5C | Industrial waste heat from beverage | 30-90 | Hot air | 35-60 |
| | | manufacturing and/or bottling | | | |
| 121A | 5C | Industrial waste heat from beverage | 30-90 | Industrial | 110-145 |
| | | manufacturing and/or bottling | | Steam | |
| 121B | 5C | Industrial waste heat from data centers | 30-90 | Low | 115-130 |
| | | | | Pressure | |
| | | | | Steam | |
| 121C | 5C | Industrial waste heat from beverage | 30-90 | Hot | 50-70 |
| | | manufacturing and/or bottling | | water | |
| 121D | 5C | Industrial waste heat from beverage | 30-90 | Hot air | 35-60 |
| | | manufacturing and/or processing | | | |
| 122A | 5C | Industrial waste heat from dairy | 30-90 | Industrial | 110-145 |
| | | manufacturing and/or processing | | Steam | |
| 122B | 5C | Industrial waste heat from dairy | 30-90 | Low | 115-130 |
| | | manufacturing and/or processing | | Pressure | |
| | | | | Steam | |
| 122C | 5C | Industrial waste heat from dairy | 30-90 | Hot | 50-70 |
| | | manufacturing and/or processing | | water | |
| 122D | 5C | Industrial waste heat from dairy | 30-90 | Hot air | 35-60 |
| | | manufacturing and/or processing | | | |
| 123A | 5D | Industrial waste heat from dairy | 30-90 | Industrial | 110-145 |
| | | manufacturing and/or processing | | Steam | |
| 123B | 5D | Industrial waste heat from dairy | 30-90 | Low | 115-130 |
| | | manufacturing and/or processing | | Pressure | |
| | | | | Steam | |
| 123C | 5D | Industrial waste heat from dairy | 30-90 | Hot | 50-70 |
| | | manufacturing and/or processing | | water | |
| 123D | 5D | Industrial waste heat from dairy | 30-90 | Hot air | 35-60 |
| | | manufacturing and/or processing | | | |
| 124A | 5D | Industrial waste heat | 30-90 | Industrial | 110-145 |
| | | | | Steam | |
| 124B | 5D | Industrial waste heat | 30-90 | Low | 115-130 |
| | | | | Pressure | |
| | | | | Steam | |
| 124C | 5D | Industrial waste heat | 30-90 | Hot | 50-70 |
| | | | | water | |
| 124D | 5D | Industrial waste heat | 30-90 | Hot air | 35-60 |
| 125A | 5D | Industrial waste heat from data centers | 30-90 | Industrial | 110-145 |
| | | | | Steam | |
| 125B | 5D | Industrial waste heat from data centers | 30-90 | Low | 115-130 |
| | | | | Pressure | |
| | | | | Steam | |
| 125C | 5D | Industrial waste heat from data centers | 30-90 | Hot | 50-70 |
| | | | | water | |
| 125D | 5D | Industrial waste heat from data centers | 30-90 | Hot air | 35-60 |
| 126A | 5D | Industrial waste heat from data centers | 30-90 | Industrial | 110-145 |
| | | | | Steam | |
| 126B | 5D | Industrial waste heat from data centers | 30-90 | Low | 115-130 |
| | | | | Pressure | |
| | | | | Steam | |
| 126C | 5D | Industrial waste heat from data centers | 30-90 | Hot | 50-70 |
| | | | | water | |
| 126D | 5D | Industrial waste heat from data centers | 30-90 | Hot air | 35-60 |
| 127A | 5D | Industrial waste heat from beverage | 30-90 | Industrial | 110-145 |
| | | manufacturing and/or bottling | | Steam | |
| 127B | 5D | Industrial waste heat from beverage | 30-90 | Low | 115-130 |
| | | manufacturing and/or bottling | | Pressure | |
| | | | | Steam | |
| 127C | 5D | Industrial waste heat from | 30-90 | Hot | 50-70 |
| | | beveragemanufacturing and/or bottling | | water | |
| 127D | 5D | Industrial waste heat from beverage | 30-90 | Hot air | 35-60 |
| | | manufacturing and/or bottling | | | |
| 128A | 5D | Industrial waste heat from beverage | 30-90 | Industrial | 110-145 |
| | | manufacturing and/or bottling | | Steam | |
| 128B | 5D | Industrial waste heat from data centers | 30-90 | Low | 115-130 |
| | | | | Pressure | |
| | | | | Steam | |
| 128C | 5D | Industrial waste heat from beverage | 30-90 | Hot | 50-70 |
| | | manufacturing and/or bottling | | water | |
| 128D | 5D | Industrial waste heat from beverage | 30-90 | Hot air | 35-60 |
| | | manufacturing and/or processing | | | |
| 129A | 5D | Industrial waste heat from dairy | 30-90 | Industrial | 110-145 |
| | | manufacturing and/or processing | | Steam | |
| 129B | 5D | Industrial waste heat from dairy | 30-90 | Low | 115-130 |
| | | manufacturing and/or processing | | Pressure | |
| | | | | Steam | |
| 129C | 5D | Industrial waste heat from dairy | 30-90 | Hot | 50-70 |
| | | manufacturing and/or processing | | water | |
| 129D | 5D | Industrial waste heat from dairy | 30-90 | Hot air | 35-60 |
| | | manufacturing and/or processing | | | |

Applicants have found that the refrigerant compositions, systems and methods of the present invention are able to satisfy in an exceptional and unexpected way the need for HTHP systems having excellent performance with respect to environmental impact while at the same time providing other important performance characteristics.

In preferred embodiments the present compositions provide alternatives and/or replacements for working fluids currently used in high temperature heat pump applications, particularly and preferably as alternatives and/or replacements for CFC-114 and R-1233zd(Z).

In preferred embodiments, each of the methods of the present invention, including Heat Transfer Method 1 and HTHP Methods 1-129, utilizes a refrigerant of the present invention, including each of Composition 1-5 and HTCs 1-6, wherein the critical temperature of the refrigerant is greater than the temperature of the high temperature heat sink, and preferably of about 150 °C to about 160 °C.

In preferred embodiments, each of the methods of the present invention, including Heat Transfer Method 1 and HTHP Methods 1-129, utilizes a refrigerant of the present invention, including each of Compositions 1-5 and HTCs 1-6, wherein the critical temperature of the refrigerant is greater than the temperature of the high temperature heat sink and wherein the refrigerant critical temperature is about 160 °C or less.

The present invention provides a method of heating a fluid or body using a high temperature heat pump, including Heat Transfer Method 1 and HTHP Methods 1-129, said method comprising the steps of (a) condensing a refrigerant composition of the present invention, including each of Compositions 1-5 and HTCs 1-6, in the vicinity of the fluid of body or be heated, and (b) evaporating said refrigerant.

Examples of high temperature heat pumps include a heat pump tumble dryer or an industrial heat pump. It will be appreciated the heat pump may comprise a suction line/liquid line heat exchanger (SL-LL HX). By “high temperature heat pump”, it is meant a heat pump that is able to generate temperatures of at least about 80 °C, preferably at least about 90 °C, preferably at least about 100 °C, more preferably at least about 110 °C.

### Methods

The present heat transfer fluids, or heat transfer compositions which can include a secondary loop air conditioning system which utilize a refrigerant of the present invention, including each of Compositions 1-5 and HTCs 1-6, may be used as a replacement for existing fluids.

The invention includes a method of replacing an existing heat transfer fluid in a heat transfer system, said method comprising the steps of (a) removing at least a portion of said existing heat transfer fluid from said system, and subsequently (b) introducing into said system a heat transfer fluid of the invention. Step (a) may involve removing at least about 5 wt. %, at least about 10 wt. %, at least about 15 wt. %, at least about 50 wt. % at least about 70 wt. %, at least about 90 wt. %, at least about 95 wt. %, at least about 99 wt. % or at least about 99.5 wt. % or substantially all of said existing heat transfer fluid from said system prior to step (b).

The method may optionally comprise the step of flushing said system with a solvent after conducting step (a) and prior to conducting step (b).

For the purposes of this invention, the heat transfer fluid of the present invention, including each of Compositions 1-5 and HTCs 1-6, can be used to replace an existing fluid in an electronic device, in an Organic Rankine cycle, or in a high temperature heat pump.

For example, the thermal management fluid of the invention, including each of Compositions 1-5, may be used as a replacement for existing fluids such as HFC-4310mee, HFE-7100 and HFE-7200. Alternatively, the thermal management fluid, including each of Compositions 1-5 and HTCs 1-6, can be used to replace water and glycol. The replacement may be in existing systems, or in new systems which are designed to work with an existing fluid. Alternatively, the thermal management fluid, including each of Compositions 1-5 and HTCs 1-6, can be used in applications in which the existing refrigerant was previously used. For example, the refrigerants of the invention, including each of Compositions 1-5 and HTCs 1-6, may be used as a replacement for existing refrigerants such as HFC-245fa, HFC-134a, HFC-404A and HFC-410A. The refrigerant, including each of Compositions 1-5 and HTCs 1-6, may be used in applications in which the existing refrigerant was previously used. Alternatively, the refrigerant of the present invention, including each of Compositions 1-5 and HTCs 1-6, may be used to retrofit an existing refrigerant in an existing system. Alternatively, the refrigerant of the present invention, including each of Compositions 1-5 and HTCs 1-6, may be used in new systems which are designed to work with an existing refrigerant.

The invention provides a method of replacing an existing refrigerant in a heat transfer system, said method comprising the steps of (a) removing at least a portion of said existing refrigerant from said system, and subsequently (b) introducing into said system a refrigerant of the invention of the present invention, including each of Compositions 1-5. The existing refrigerants may be selected, for example, from HFC-245fa, HFC-134a, HFC-404A and HFC-410A.

Step (a) may involve removing at least about 5 wt. %, at least about 10 wt. %, at least about 15 wt. %, at least about 50 wt. % at least about 70 wt. %, at least about 90 wt. %, at least about 95 wt. %, at least about 99 wt. % or at least about 99.5 wt. % of said existing refrigerant from said system prior to step (b).

The method may optionally comprise the step of flushing said system with a solvent after conducting step (a) and prior to conducting step (b).

### Examples

### Example 1A-Organic Rankine Cycle-Using Compound 1

This example illustrates that a refrigerant consisting of 100% of Compound 1 (Boiling Point 51 °C) is useful as a working fluid in an Organic Rankine cycle based on a comparison of the estimated thermal efficiency of various working fluids in an organic Rankine cycle. In this example, an ORC system is assumed to contain a condenser, pump, boiler and turbine and the following qualitative results will occur as shown in Table Ex1A below.

TABLE Ex1A

| | | Process Specifications | | | | | | |
|---|---|---|---|---|---|---|---|---|
| | | | | Boiler | | | | |
| | | Boiler | Critical | Super- | Isentropic | Condensing | Isentropic | Estimated |
| | Working | Temp | Temp | heat | Efficiency | Temp | Efficiency | Thermal |
| | Fluid | (°C) | (°C) | (°C) | (Turbine) | (°C) | (Pump) | Efficiency |
| Ex. | Compound 1- | 144 | 225 | 1 | 0.8 | 35 | 0.8 | 15-20% |
| 1A | 100% of | | | | | | | |
| | Refrigerant | | | | | | | |
| | R245fa | 144 | 154 | 1 | 0.8 | 35 | 0.8 | 15.41% |
| | R1233zd(E) | 144 | 166 | 1 | 0.8 | 35 | 0.8 | 15.92% |
| Ex. | Compound 1 | 210 | 225 | 1 | 0.8 | 35 | 0.8 | 15-20% |
| 1B | R245fa | 144 | 154 | 1 | 0.8 | 35 | 0.8 | 15.41% |
| | R1233zd(E) | 156 | 166 | 1 | 0.8 | 35 | 0.8 | 16.66% |

### Example 1B—Organic Rankine Cycle-Using Each of Compositions 1-5 and HTCs 1-6

Example 1A is repeated, except that each of the compositions within Compositions 1-5 and HTCs 1-6 are used in place of 100% Compound 1 as the refrigerant. Similar advantageous results are achieved.

### Example 2A-Battery Cooling-Single Phase Via Heat Exchanger-Using Compound 1

Batteries of electric vehicles develop heat during operation when charging and discharging. The typical design of vehicle batteries differs between three types: cylindrical cells, pouch cells and prismatic cells. All three types have different considerations in terms of heat transfer due to their shape. Prismatic and pouch cells are often used with cooling plates due to the straight outer faces. Cylindrical cells employ cooling ribbons that are in thermal contact with the outer shell of the cells. Extensive heat generation during charging and discharging of the cells can lead to an increase in temperature that can cause decreasing performance and reduced battery lifetime.

A battery cooling plate set up may be used to provide active cooling to a battery and remove the heat (e.g. to remove heat from the battery of an electric vehicle). In this Example, the performance of a heat transfer composition comprising 100% of Compound 1 and 3M Novec 7200 is analyzed for its ability to provide cooling in single phase heat transfer.

It will be appreciated that the convective heat transfer can occur either by direct contact, ie. when the battery is immersed in the fluid that may be pumped through the battery enclosure or indirectly, i.e. by using a cooling plate with a combination of convective and conductive heat transfer.

The present example uses a round tube with an internal diameter of 0.55 inches to provide a cooling load of 10246 BTU/h (3 kW). The tube length was 30 ft (9.14 m) with an assumed pressure drop of 2.9PSI (20 kPa). The fluid temperature was 7.2 C (45F). The internal heat transfer coefficient is determined for turbulent flow. The necessary mass flow rate to remove the cooling load is determined for both fluids. The results of the comparison are shown in the Table Ex2A below. It can be seen in the results that the necessary mass flow rate to remove the generated heat is about or less than for 3M Novec 7200 and that the useful output (I.e. the heat transfer coefficient) is about or higher than 3M Novec 7200.

TABLE Ex2A
Heat Transfer and Pressure Drop For Heat Exchanger Set Up

| | Mass | Prandtl | Internal heat |
|---|---|---|---|
| | Flow Rate | Number | transfer coefficient |
| | lb/s | [—] | BTU/(h-ft²-F) |
| Compound 1 | 0.9-1 | 9-11 | 300-350 |
| 3M Novec 7200 | 0.98 | 10.4 | 303.4 |

### Example 2B—Battery Cooling—Single Phase Via Heat Exchanger—Using Each of Compositions 1-5 and HTCs 1-6

Example 2A is repeated, except that each of the compositions within Compositions 1-5 and HTCs 1-6 are used in place of 100% Compound 1 as the refrigerant. Similar advantageous results are achieved.

### Example 3A—Battery Cooling Vis Sensible Heat Immersion Cooling Using Compound 1

Batteries of electric vehicles develop heat during operation when charging and discharging. The typical design of vehicle batteries differs between three types: Cylindrical cells, pouch cells and prismatic cells. All three types have different considerations in terms of heat transfer due to their shape. Extensive heat generation during charging and discharging of the cells can lead to an increase in temperature that can cause decreasing performance and reduced battery lifetime.

Compound 1 of the present invention (boiling point 51 C) has a low dielectric constant, high dielectric strength, and is as nonflammable fluid, which allows for direct cooling of the battery cells that are immersed therein.

The present example considers a battery module that consists of 1792 cylindrical battery cells of 18650 type. In one case the battery module is cooled by a 50/50 mixture of water/glycol in a flat tube heat exchanger that is on contact with the battery cells. In the other case the cells are immersed in Compound 1, i.e. are in direct contact with the fluid. The waste heat for the battery module is 8750 W that is evenly distributed over the total number of cells. The assumptions and operating conditions are listed in Table Ex3.

TABLE Ex3A
Assumptions for Battery module design and operating conditions

| | | | Compo- |
|---|---|---|---|
| | | | sitions |
| Parameter | Unit | Water/Glycol | 1-38 |
| Battery diameter | [mm] | 18.5 | 18.5 |
| Battery gap | [mm] | 3.8 | 1.5 |
| Battery height | [mm] | 65 | |
| Number of batteries | [—] | 1792 | |
| Battery mass | [g] | 49 | |
| Battery specific heat | [J/kgK] | 830 | |
| Total battery module waste heat | [W] | 8750 | |
| Fluid flow rate | [kg/s] | 0.1 | |
| Initial module temperature | [°C] | 30 | |
| Fluid inlet temperature | [°C] | 10 | |
| Cooling channel height | [mm] | 30 | n/a |
| Cooling channel width | [mm] | 2.8 | n/a |
| Heat exchanger flat tube wall | [mm] | 0.5 | n/a |
| thickness | | | |
| Heat exchanger flat tube | [W/mK] | 3 | n/a |
| thermal conductivity | | | |
| Heat exchanger flat tube | [—] | 0.0003 | n/a |
| relative surface roughness | | | |

TABLE Ex3B
Minimum and maximum cell temperatures in battery module

| | Minimum cell temperature [°C] | | Maximum cell temperatures [°C] | |
|---|---|---|---|---|
| | Water/Glycol | Com- | Water/Glycol | Com- |
| Time | 50/50 | pound 1 | 50/50 | pound 1 |
| 0 | 30.0 | 30.0 | 30.0 | 30.0 |
| 100 | 35.8 | 10-40 | 36.8 | 30-40 |
| 200 | 40.3 | 10-40 | 42.0 | 30-40 |
| 300 | 43.6 | 10-45 | 46.0 | 30-45 |
| 400 | 46.1 | 10-45 | 49.2 | 30-50 |
| 500 | 48.0 | 10-45 | 51.7 | 30-50 |
| 600 | 49.5 | 10-50 | 53.6 | 30-55 |
| 700 | 50.5 | 10-50 | 55.1 | 30-55 |
| 800 | 51.4 | 10-50 | 56.3 | 30-55 |
| 900 | 52.0 | 10-50 | 57.2 | 30-55 |

### Example 3B—Battery Cooling Vis Sensible Heat Immersion Cooling—Using Each of Compositions 1-5 and HTCs 1-6

Example 3A is repeated, except that each of the compositions within Compositions 1-5 and HTCs 1-6 are used in place of 100% Compound 1 as the refrigerant. Similar advantageous results are achieved.

### Example 4A—Two Phase Immersion Cooling in a Data Center Using Compound 1

An example of data center cooling is provided, making reference to FIG. 4. A data center, generally denoted 200, includes a plurality of electronic subsystems 220 contained in one or more of electronics racks 210. At least one, and preferably a plurality, and preferably all, of the electronic subsystems 220 are associated with a cooling station 240 that includes (in one embodiment) a vertically-extending, liquid-to-air heat exchanger 243 and supply and return ducting 241, 242 for directing a cooling airflow 244 across liquid-to-air heat exchanger 243. A cooling subsystem 219 is associated with at least one, and preferably a plurality, and preferably all, of the multiple electronic subsystems 220. In a preferred embodiment, as shown in FIG. 4, all of the subsystem 220 are associated with the cooling station 240 and a cooling subsystem 219. Each cooling subsystem 219 comprises (in this embodiment) a housing 221 (which preferably is a low pressure housing) which encloses a respective electronic subsystem 220 comprising a plurality of electronic components 223. The electronic components are in operation as part of the data center and are generating heat as a result of performing their function in the data center. The components include, by way of example, printed circuit boards, microprocessor modules, and memory devices. Each electronic subsystem has, as it is operating, its heat generating components immersed in a thermal management fluid consisting of Compound 1 of the present invention. The fluid 224 boils in typical operation, generating dielectric vapor 225 according to the present invention. In the illustrated embodiment, electronic subsystems 220 are angled by providing upward-sloped support rails 222 within electronics rack 210 to accommodate the electronic subsystems 220 at an angle. Angling of the electronic subsystems as illustrated facilitates buoyancy-driven circulation of vapor 225 between the cooling subsystem 219 and the liquid-to-air heat exchanger 243 of the associated local cooling station 240. However, the excellent results according to the present invention and the present example are achieved equally well when such angling is not used. Multiple coolant loops 226 are coupled in fluid and thermal contact with the liquid-cooled electronic subsystems and a respective portion of liquid-to-air heat exchanger 243. In particular, multiple tubing sections 300 pass through liquid-to-air heat exchanger 243, which in this example includes a plurality of air-cooling fins 310. Vapor 225 is buoyancy-driven from housing 221 to the corresponding tubing section 300 of liquid-to-air heat exchanger 243, where the vapor condenses and is then returned as liquid to the associated liquid-cooled electronics subsystem. Cooling airflow 244 is provided in parallel to the supply ducting 241 of multiple local cooling stations 240 of data center 200, and the heated airflow is exhausted via return ducting 242. The equipment as described herein, but not the fluid of the present invention, is disclosed in US 2013/0019614, which is incorporated herein by reference.

The system as describe above is operated with a thermal management fluid consisting of Compound 1, and ambient air as the heat sink for the condenser, and this system operates to effectively, efficiently, safely and reliably maintain the electronic components in the most desired operating temperature range while the system is performing its function in the operating data center.

### Example 4B—Two Phase Immersion Cooling in a Data Center—Using Each of Compositions 1-5 and HTCs 1-6

Example 4A is repeated, except that each of the compositions within Compositions 1-5 and HTCs 1-6 are used in place of 100% Compound 1 as the refrigerant. Similar advantageous results are achieved.

### Example 5A—High Temperature Heat Pump Using Compound 1

High temperature heat pumps can utilize waste heat and provide high heat sink temperatures. Compound 1 of the present invention (boiling point 51 C) each provide efficiency equal to about or superior to R245fa over a wide range of condensing temperatures. Results are shown in Table Ex5.

### Operating Conditions:

Condensing temperature varied between 90 °C, 100 °C and 110 °C
Subcooling: 10 °C
Evaporating temperature: 25 °C
Evaporator Superheat: 15 °C
Isentropic efficiency: 65%

TABLE Ex5
Relative heating COP at varying condensing temperatures

| Fluid | Condensing temperature | | |
|---|---|---|---|
| Fluid | 90 °C | 100 °C | 110 °C |
| R245fa | 100.0% | 100.0% | 100.0% |
| Compound 1 | 100-105% | 100-105% | 100-105% |

### Example 5B—High Temperature Heat Pump-Using Each of Compositions 1-5 and HTCs 1-6

Example 5A is repeated, except that each of the compositions within Compositions 1-5 and HTCs 1-6 are used in place of 100% Compound 1 as the refrigerant. Similar advantageous results are achieved.

### Example 6—Synthesis of Compound 1—ethynylpentafluoro-λ⁶-sulfane

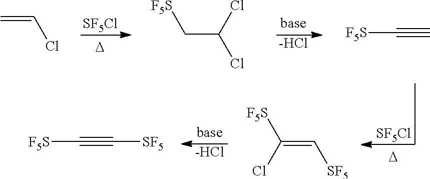

Compound 1 is formed using a four step process.

In step 1, a 300 mL Hastelloy® autoclave is sequentially charged with vinyl chloride (0.25 mol, 15.6 g) and sulfur chloride pentafluoride (0.25 mol, 40.6 g). With vigorous stirring (~1200 rpm), the reaction mixture is heated to 160 °C and held at this temperature for about 20 hours. The unreacted vinyl chloride and sulfur chloride pentafluoride are transferred into a cylinder for reuse, and the liquid product is left behind in the autoclave. The liquid product containing (2,2-dichloroethyl) pentafluoro-λ⁶-sulfane is washed with water, dried over MgSO₄ and used as is for step 2.

In step 2, a four-neck, 500 mL round bottom flask equipped with an overhead stirrer, a thermocouple, an addition funnel, and a PFA line connected to a dry ice/isopropanol trap is charged with 200 mL of 50 wt % aq. NaOH solution and benzyltrimethylammonium chloride (5 g). The resultant suspension is heated to about 80 °C. The (2,2-dichloroethyl) pentafluoro-λ⁶-sulfane (0.25 mol, 56.3 g) product from step 1 is added dropwise to the hot solution. The desired product of this step 2, ethynylpentafluoro-26-sulfane, is evolved as the addition proceeds and is continually collected in the dry ice trap. The collected product is degassed using liquid nitrogen and used as is in step 3.

In step 3, a 300 mL Hastelloy® autoclave is sequentially charged with ethynylpentafluoro-λ⁶-sulfane (0.25 mol, 38.0 g) from step 2 and sulfur chloride pentafluoride (0.25 mol, 40.6 g). With vigorous stirring (~1200 rpm), the reaction mixture is heated to about 160 °C and held at this temperature for about 20 hours. The unreacted ethynylpentafluoro-26-sulfane and sulfur chloride pentafluoride are transferred into a cylinder for reuse, and the liquid product containing (1-chloroethane-1,2-diyl)bis(pentafluoro-26-sulfane) is left behind in the autoclave. The liquid product is washed with water, dried over MgSO₄ used in as is for step 4.

In step 4, a four-neck, 500 mL round bottom flask equipped with an overhead stirrer, a thermocouple, an addition funnel, and a distillation apparatus (consisting of a Vigreux column attached to a PFA line) terminating in a 100 mL round bottom flask cooled in a dry ice/isopropanol bath, is charged with 200 mL of 50 wt % aq. NaOH solution and benzyltrimethylammonium chloride (5 g). The resultant suspension is heated up to 100 °C. while stirring. The (1-chloroethane-1,2-diyl)bis(pentafluoro-λ⁶-sulfane) (0.25 mol, 56.3 g) made in step 3 is added dropwise to the aforementioned hot suspension. The desired product ethynylpentafluoro-26-sulfane is evolved as a gas as the addition proceeds and is continually collected in the cooled 100 mL round bottom flask.

### Example 7—Synthesis of Compound 2-(E)-1,2-Bis(Pentafluoro-λ⁶-Sulfaneyl) Ethene

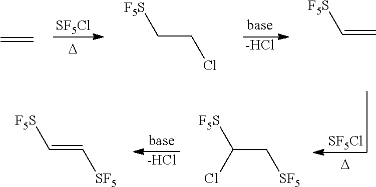

Compound 2 is formed using a four step process.

In step 1, a 300 mL Hastelloy autoclave reactor pressure checked at 2300 psi is evacuated, cooled in liquid N₂, and then charged sequentially with ethylene (11.2 g, 1.0 equiv, 0.40 mol) and sulfur chloride pentafluoride (65.0 g, 1.0 equiv, 0.40 mol). The pressure is monitored while warming to room temperature, at which point the stirring is started. The vessel is then heated to a temperature between 100 °C and 300 °C for 4 hour or until a minimum pressure is reached and remains constant for about 1 hour. Once the reaction is deemed complete, the autoclave is cooled to about 0 °C. The gaseous materials (mostly residual ethylene and SF₅Cl) are then transferred into an evacuated cylinder. The autoclave is then opened. The liquid material is collected, washed with water, dried over MgSO₄, and passed through a filter into a glass storage vessel. The crude (2-chloroethyl) pentafluoro-26-sulfane is used as-is without further purification in step 2.

In step 2, a 500 mL 4-neck RB flask equipped with mechanical stirrer, liquid addition funnel, internal temperature probe, and condenser apparatus (see details below) is charged with benzyltrimethylammonium chloride (3.7 g, 0.05 equiv, 0.02 mol) and 50 wt % aqueous sodium hydroxide (320 g, 213 ml, 20 equiv, 8 mol) and then heated to about 60 °C in an oil bath while stirring. The addition funnel is charged with the liquid material from step 1, (2-chloroethyl) pentafluoro-26-sulfane (76.2 g, 1.0 equiv, 0.4 mol), which is then added to the stirred pot at a rate to maintain the pot temperature at or below 70 °C. The product vapors pass through a 50 °C reflux condenser and into a distillation head with this second condenser maintained at 0 °C. The condensed liquid is flowed into a product collection vial or flask cooled to −78 °C with dry ice to prevent evaporation. The collected liquid pentafluoro (vinyl)-λ⁶-sulfane is used as is without further purification in step 3.

In step 3, a 300 mL Hastelloy autoclave reactor pressure checked at 2300 psi is evacuated, cooled in liquid N₂, and then charged sequentially with the product from step 2, pentafluoro (vinyl)-λ⁶-sulfane (61.6 g, 1.0 equiv, 0.40 mol), and sulfur chloride pentafluoride (65.0 g, 1.0 equiv, 0.40 mol). The pressure is monitored while warming to room temperature, at which point the stirring is started. The vessel is then heated to a temperature between 100 °C and 300 °C for about 4 hours or until a minimum pressure is reached and remains constant for about 1 hour. Once the reaction is deemed complete, the autoclave is cooled to about 0 °C. The gaseous materials (mostly residual SF₅Cl) are then transferred into an evacuated cylinder. The autoclave is then opened. The liquid material is collected, washed with water, dried over MgSO₄, and passed through a filter into a glass storage vessel. The crude (1-chloroethane-1,2-diyl)bis(pentafluoro-26-sulfane) is used as is without further purification in step 4.

In step 4, a 500 mL 4-neck RB flask equipped with mechanical stirrer, liquid addition funnel, internal temperature probe, and condenser apparatus (as described below) is charged with benzyltrimethylammonium chloride (3.7 g, 0.05 equiv, 0.02 mol) and 50 wt % aqueous sodium hydroxide (320 g, 213 ml, 20 equiv, 8 mol) and then heated to about 80 °C in an oil bath while stirring. The addition funnel is charged with the liquid material from step 3, (1-chloroethane-1,2-diyl)bis(pentafluoro-26-sulfane) (126.6 g, 1.0 equiv, 0.4 mol), which is then added to the stirred pot at a rate to maintain the pot temperature at or below about 85 °C. The product vapors pass through a 90 °C reflux condenser and into a distillation head with this second condenser maintained at 0 °C. The condensed liquid is flowed into a product collection vial or flask cooled to −78 °C with dry ice to prevent evaporation. The collected liquid (E)-1,2-bis(pentafluoro-λ⁶-sulfaneyl) ethene is then purified by distillation.

### Example 8—Synthesis of Compound 3-1,2-Bis(Pentafluoro-26-Sulfaneyl)Ethane

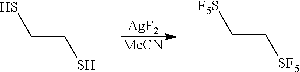

To an oven-dried 500 mL round bottom flask is added AgF2 (10 equiv, 365 g) and acetonitrile (200 mL) under an atmosphere of N2. Using an addition funnel, ethane-1,2-dithiol (0.25 mol, 23.5 g) is added dropwise to the reaction mixture such that the temperature does not rise above about 40 °C. The suspension is then stirred for about 1 hour at room temperature and monitored for completion by GC-MS. Then, hexanes (30 mL) are added to the solution, stirred for 10 mins, allowed to settle, and then decanted into a new flask. This is repeated two more times. The desired compound is obtained by removing hexanes using a rotary evaporator, followed by a distillation of the desired product, 1,2-bis(pentafluoro-26-sulfaneyl) ethane, to remove from heavy impurities.

What is claimed is:

1. A heat transfer composition for transferring heat and/or energy to and/or from an article, device or fluid, wherein said heat transfer composition comprises one or more compounds of Formula I:
F₅S—C₂H_{X}—SF₅ Formula I
where x is 0, 2 or 4.

2. The heat transfer composition of claim 1 wherein said heat transfer composition consists essentially of one or more compounds of said Formula I.

3. The heat transfer composition of claim 1 wherein said heat transfer composition consists of one or more compounds of said Formula I.

4. The heat transfer composition of claim 1 wherein said heat transfer composition comprises a compound according to Formula IA:
F₅S—C≡C—SF₅ Formula IA.

5. The heat transfer composition of claim 1 wherein said heat transfer composition comprises a compound according to Formula IB:
F₅S—CH═CH—SF₅ Formula IB.

6. The heat transfer composition of claim 1 wherein said heat transfer composition comprises a compound according to Formula IB (trans):

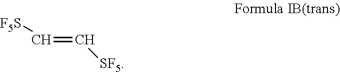

7. The heat transfer composition of claim 1 wherein said heat transfer composition comprises a compound according to Formula IB(cis):

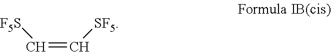

8. The heat transfer composition of claim 1 wherein said heat transfer composition comprises a compound according to Formula IC:
F₅S—CH₂—CH₂—SF₅ Formula IC.

9. A method for transferring heat and/or energy from an article, device or fluid comprising:
(a) providing the article, device or fluid; and
(b) transferring heat and/or energy between a composition of according to claim 1.

10. A heat transfer system containing a heat transfer composition according to claim 1.

11. The heat transfer composition of claim 1 having a wherein said heat transfer composition has a boiling point of from about 50 °C to about 250 °C.

12. The heat transfer composition of claim 1 having a wherein said heat transfer composition has a boiling point of from about 50 °C to about 200 °C.

13. The heat transfer composition of claim 1 having a wherein said heat transfer composition has a boiling point of from about 50 °C to about 150 °C.

14. The heat transfer composition of claim 11 wherein said heat transfer composition has a dielectric constant of less than about 5.

15. The heat transfer composition of claim 11 wherein said heat transfer composition has a dielectric constant of less than about 2.5.

16. The heat transfer composition of claim 11 wherein said heat transfer composition has a dielectric constant of from about 1.5 to about 3.

17. The heat transfer composition of claim 11 wherein said compound(s) of Formula I are present in an amount of at least about 50% by weight of the composition.

18. The heat transfer composition of claim 11 wherein said compound(s) of Formula I are present in an amount of at least about 70% by weight of the composition.

19. The heat transfer composition of claim 11 wherein said heat transfer composition has a GWP of not greater than about 150.

20. The heat transfer composition of claim 11 wherein said heat transfer composition further comprises a lubricant.
